(19) United States
(12) Patent Application Publication
(10) Pub. No.: US 2026/0083845 A1
(43) Pub. Date: Mar. 26, 2026

(54) ENGINEERED IMMUNE CELLS

(71) Applicant: Cellicure, Inc., Germantown, MD (US)

(72) Inventors: Minggang Zhang, Germantown, MD (US); Belle X. Wang, Germantown, MD (US); Yongmei Li, Germantown, MD (US); Nathan X. Li, Germantown, MD (US); Qisheng Li, Germantown, MD (US)

(73) Assignee: Cellicure, Inc., Germantown, MD (US)

(21) Appl. No.: 19/112,644

(22) PCT Filed: Sep. 22, 2023
(86) PCT No.: PCT/US2023/074830

§ 371 Date: Mar. 18, 2025

Related U.S. Application Data
(60) Provisional application No. 63/376,931, filed on Sep. 23, 2022.

(51) Int. Cl. A61K 40/15; A61K 40/31; A61K 40/42; A61P 35/00; C07K 14/725; C07K 16/28

(52) U.S. Cl. CPC A61K 40/15; A61K 40/31; A61K 40/4211; A61P 35/00; C07K 14/7051; C07K 16/2803; A61K 2239/22; C07K 2317/622

(57) ABSTRACT

The present application relates to chimeric antigen receptors (CARs), engineered immune cells (e.g., NK cells, NKT cells, and T cells), and methods of use thereof. The CARs provided herein can enhance the killing activities of the engineered NK cells.

### CROSS REFERENCE

This application claims the benefit of U.S. Provisional Application No. 63/376,931, filed Sep. 23, 2022, the full disclosure of which is hereby incorporated by reference herein in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14767-001-228_SEQLISTING.xml”, was created on Sep. 10, 2023 and is 215,150 bytes in size.

### 1. INTRODUCTION

The present application relates to chimeric antigen receptors (CARs), engineered immune cells (e.g., NK cells, NKT cells, and T cells), and methods of use thereof. The CARs provided herein can enhance the killing activities of the engineered NK cells.

### 2. BACKGROUND

Immunotherapy has become a promising approach to treat cancer. T cells expressing chimeric antigen receptor (CAR) have demonstrated to be effective for liquid tumors, but with very limited effect against solid tumors (Porter et al. (2011) Chimeric antigen receptor-modified T cells in chronic lymphoid leukemia. N Engl J Med. 365(8):725-33; Ahmed et al. (2015) Autologous HER2 CMV bispecific CAR T cells are safe and demonstrate clinical benefit for glioblastoma in a Phase I trial. J Immunother Cancer. 3(Suppl 2):011; Fesnak et al. (2016) Engineered T cells: the promise and challenges of cancer immunotherapy. Nat Rev Cancer. 16(9):566-81).

Natural killer (NK) cells are essential part in innate immune system and constitute the first defense line against tumor (Campbell et al. (2013) Natural killer cell biology: an update and future directions. J Allergy Clin Immunol. 132(3):536-544). CAR engineered NK cells (CAR-NK) have become more attractive due to the unique biological property of NK cells. Firstly, CAR-NK has higher safety compared to CAR-T, including the lower risk of cytokine release syndrome (CRS) and neurotoxicity, lower risk of graft-versus-host disease (GVHD)(Liu et al. (2020) Use of CAR-Transduced Natural Killer Cells in CD19-Positive Lymphoid Tumors. N Engl J Med. 382(6):545-553; Lupo et al. (2019) Natural Killer Cells as Allogeneic Effectors in Adoptive Cancer Immunotherapy. Cancers (Basel). 11(6):769). Secondly, multifunctionality of CAR-NK can kill target tumor cells in CAR-specific manner. In addition, NK cells exhibit potent cytotoxic activity against tumor cells through many constitutively expressed activating receptors such as NCRs, NKG2D, DNAM-1, activating KIRs (KIR2DS1, KIR2DS4, KIR2DL4), CD16-mediated antibody-dependent cellular cytotoxicity (ADCC), and TRAIL or FasL induced apoptosis processes (Oei et al. (2018) Intrinsic Functional Potential of NK-Cell Subsets Constrains Retargeting Driven by Chimeric Antigen Receptors. Cancer Immunol Res. 6(4):467-480; Sun et al. (2015) NK cell receptor imbalance and NK cell dysfunction in HBV infection and hepatocellular carcinoma. Cell Mol Immunol. 12(3):292-302; Wu et al. (2019) Role of ADAM17 as a regulatory checkpoint of CD16A in NK cells and as a potential target for cancer immunotherapy. J Leukoc Biol. 105(6):1297-1303). Thirdly, CAR-NK cells can provide an off-the-shelf product, eliminating the need for a personalized and patient-specific product. The minimal risk for alloreactivity and GVHD potentially permits allogeneic CAR-NK cells to be procured from various sources, including NK cell line, peripheral blood mononuclear cell (PBMC), and pluripotent stem cell (iPSC)(Shimasaki et al. (2020) NK cells for cancer immunotherapy. Nat Rev Drug Discov. 19(3):200-218; Ueda et al. (2020) Non-clinical efficacy, safety and stable clinical cell processing of induced pluripotent stem cell-derived anti-glypican-3 chimeric antigen receptor-expressing natural killer/innate lymphoid cells. Cancer Sci. 111(5):1478-1490).

The most of the current NK CARs, including the CAR-NKs investigated in the latest clinical trials, use T cell derived CARs, such as ScFv-CD8a hinge-CD28 TM and ICD-CD3zeta. It has been reported that NK specific CAR is better than T CAR expressed in NK cells (Li et al. (2018) Human iPSC-Derived Natural Killer Cells Engineered with Chimeric Antigen Receptors Enhance Anti-tumor Activity. Cell Stem Cell. 23(2):181-192.e5).

### 3. SUMMARY OF THE INVENTION

In one aspect, provided herein are chimeric antigen receptors (CARs). In certain embodiments, provided herein is a chimeric antigen receptor (CAR), comprising:
(i) an extracellular antigen binding domain;
(ii) a transmembrane domain; and
(iii) an intracellular signaling domain comprising an intracellular domain derived from a first polypeptide,
wherein the intracellular domain derived from the first polypeptide is linked to the C-terminus of the transmembrane domain directly or via a peptide linker, and
wherein the first polypeptide is DAP10 or DAP12.

In certain embodiments, provided herein is a chimeric antigen receptor (CAR), comprising:
(i) an extracellular antigen binding domain;
(ii) a transmembrane domain comprising a transmembrane domain derived from a second polypeptide; and
(iii) an intracellular signaling domain comprising an intracellular domain derived from a first polypeptide,
wherein the second polypeptide is different from the first polypeptide,
wherein the intracellular domain derived from the first polypeptide is linked to the C-terminus of the transmembrane domain derived from the second polypeptide directly or via a peptide linker, and wherein the first polypeptide is DNAM1.

In certain embodiments, the transmembrane domain of the CAR comprises a transmembrane domain derived from a second polypeptide, wherein the second polypeptide is different from the first polypeptide, and wherein the intracellular domain derived from the first polypeptide is linked to the C-terminus of the transmembrane domain derived from the second polypeptide directly or via a peptide linker.

In certain embodiments, the intracellular signaling domain of the CAR further comprises a intracellular domain derived from a third polypeptide, wherein the third polypeptide is different from the first polypeptide.

In certain embodiments, the second polypeptide and the third polypeptide is the same polypeptide.

In certain embodiments, the CAR further comprises a hinge domain between the C-terminus of the extracellular antigen binding domain and the N-terminus of the transmembrane domain.

In certain embodiments, the hinge domain of the CAR comprises a hinge domain derived from the second polypeptide.

In certain embodiments, the second polypeptide or the third polypeptide is a signaling lymphocytic activation molecule family (SLAMF) receptor, CD2, CD28H, DNAM1, CD28, 4-1BB, OX40, KIR-S, NKG2C, or NKG2E. In certain embodiments, the SLAMF receptor is SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), or SLAMF9 (CD84H1, SF2001). In certain embodiments, the KIR-S is KIR2DS1, KIR2DS2, KIR2DS3, KIR2DS4, KIR2DS5 or KIR3DS1.

In certain embodiments, the intracellular signaling domain of the CAR further comprises an intracellular domain derived from CD3zeta.

In certain embodiments, the intracellular signaling domain of the CAR further comprises an intracellular domain derived from SLAM-associated protein (SAP). In certain embodiments, the intracellular signaling domain of the CAR further comprises an intracellular domain derived from Ewing's sarcoma-activated transcript-2 (EAT2).

In certain embodiments, the intracellular domain derived from CD3zeta is linked to the intracellular domain derived from SAP or the intracellular domain derived from EAT2 directly or via a peptide linker. In certain embodiments, the intracellular domain derived from CD3zeta is linked to the intracellular domain derived from SAP or the intracellular domain derived from EAT2 via P2A, T2A, or E2A.

In certain embodiments, the extracellular antigen binding domain of the CAR comprises an scFv. In certain embodiments, the extracellular antigen binding domain of the CAR binds to a tumor antigen or a fragment thereof.

In certain embodiments, the first polypeptide is DAP10.

In certain embodiments, the CAR comprises an amino acid sequence at least 90%, at least 95%, or 100% identical to the amino acid sequence of SEQ ID NO:20.

In certain embodiments, the CAR comprises an amino acid sequence at least 90%, at least 95%, or 100% identical to the amino acid sequence of SEQ ID NO:86.

In certain embodiments, the CAR comprises an amino acid sequence at least 90%, at least 95%, or 100% identical to the amino acid sequence of SEQ ID NO:87.

In certain embodiments, the CAR comprises an amino acid sequence at least 90%, at least 95%, or 100% identical to the amino acid sequence of SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, or SEQ ID NO:145.

In certain embodiments, the CAR, from N-terminus to C-terminus, comprises
(i) an extracellular antigen binding domain;
(ii) a transmembrane domain comprising an transmembrane domain derived from the second polypeptide;
(iii) an intracellular signaling domain comprising
   (iii)-(a) an intracellular domain derived from DAP10;
   (iii)-(b) an intracellular domain derived from CD3zeta; and
   (iii)-(c) an intracellular domain derived from SAP or an intracellular domain derived from EAT2, wherein the intracellular domains within the intracellular signaling domain are linked to each other directly or via one or more peptide linkers, and wherein the extracellular antigen binding domain, the transmembrane domain, and the intracellular domain are linked to each other directly or via one or more peptide linkers.

In another aspect, provided herein are engineered immune cells. In certain embodiments, provided herein is an engineered immune cell comprising the CAR provided herein, wherein the engineered immune cell is an NK cell, an NKT cell, or a T cell.

In certain embodiments, provided herein is an engineered immune cell comprising a CAR, wherein the CAR comprises:
(i) an extracellular antigen binding domain;
(ii) a transmembrane domain; and
(iii) an intracellular signaling domain, and wherein the engineered immune cell further comprises an exogenously introduced polynucleotide encoding an intracellular domain derived from SLAM-associated protein (SAP), and wherein the engineered immune cell is an NK cell, an NKT cell, or a T cell.

In certain embodiments, provided herein is an engineered immune cell comprising a CAR, wherein the CAR comprises:
(i) an extracellular antigen binding domain;
(ii) a transmembrane domain; and
(iii) an intracellular signaling domain, and
wherein the engineered immune cell further comprises an exogenously introduced polynucleotide encoding an intracellular domain derived from Ewing's sarcoma-activated transcript-2 (EAT2), and wherein the engineered immune cell is an NK cell, an NKT cell, or a T cell.

In certain embodiments, the polynucleotide encodes the CAR in the engineered immune cell, and wherein the intracellular signaling domain of the CAR in the engineered immune cell comprises the intracellular domain derived from SAP or the intracellular domain derived from EAT2.

In certain embodiments, the intracellular signaling domain of the CAR in the engineered immune cell comprises an intracellular domain derived from a first polypeptide, and wherein the first polypeptide is DAP10, DNAM1, CD28H, CD2, DAP12, CD28, or OX40.

In certain embodiments, the intracellular domain of the first polypeptide is linked to the C-terminus of the transmembrane domain directly or via a peptide linker.

In certain embodiments, the transmembrane domain of the CAR in the engineered immune cell comprises a transmembrane domain derived from a second polypeptide.

In certain embodiments, the intracellular signaling domain of the CAR in the engineered immune cell comprises a intracellular domain derived from a third polypeptide.

In certain embodiments, the second polypeptide or the third polypeptide is a signaling lymphocytic activation molecule family (SLAMF) receptor, CD2, CD28H, DNAM1, CD28, 4-1BB, OX40, KIR-S, NKG2C, or NKG2E. In certain embodiments, the SLAMF receptor is SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), or SLAMF9 (CD84H1, SF2001). In certain embodiments, the KIR-S is KIR2DS1, KIR2DS2, KIR2DS3, KIR2DS4, KIR2DS5 or KIR3DS1.

In certain embodiments, intracellular signaling domain of the CAR in the engineered immune cell comprises an intracellular domain derived from CD3zeta.

In certain embodiments, intracellular signaling domain of the CAR in the engineered immune cell further comprises an intracellular domain derived from SLAM-associated protein (SAP) or an intracellular domain derived from Ewing's sarcoma-activated transcript-2 (EAT2), and wherein the intracellular domain derived from CD3zeta is linked to the intracellular domain derived from SAP or the intracellular domain derived from EAT2 directly or via a peptide linker. In certain embodiments, intracellular domain derived from CD3zeta is linked to the intracellular domain derived from SAP or the intracellular domain derived from EAT2 via P2A, T2A, or E2A.

In certain embodiments, extracellular antigen binding domain of the CAR in the engineered immune cell comprises an scFv. In certain embodiments, extracellular antigen binding domain of the CAR binds to a tumor antigen or a fragment thereof.

In certain embodiments, the CAR in the engineered immune cell comprises an amino acid sequence at least 90%, at least 95%, or 100% identical to the amino acid sequence of SEQ ID NO:20.

In certain embodiments, the CAR in the engineered immune cell comprises an amino acid sequence at least 90%, at least 95%, or 100% identical to the amino acid sequence of SEQ ID NO:86.

In certain embodiments, the CAR in the engineered immune cell comprises an amino acid sequence at least 90%, at least 95%, or 100% identical to the amino acid sequence of SEQ ID NO:87.

In certain embodiments, the CAR in the engineered immune cell comprises an amino acid sequence at least 90%, at least 95%, or 100% identical to the amino acid sequence of SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, or SEQ ID NO:145.

In certain embodiments, the engineered immune cell is NK cell.

In yet another aspect, provided herein are polypeptides. In certain embodiments, provided herein is a polypeptide comprising the CAR provided herein.

In certain embodiments, provided herein is a polypeptide comprising a chimeric antigen receptor (CAR) and an intracellular domain derived from SLAM-associated protein (SAP), wherein the CAR comprises:
(i) an extracellular antigen binding domain;
(ii) a transmembrane domain; and
(iii) an intracellular signaling domain, and
wherein the CAR is linked to an intracellular domain derived from SAP via a peptide linker.

In certain embodiments, provided herein is a polypeptide comprising a chimeric antigen receptor (CAR) an intracellular domain derived from Ewing's sarcoma-activated transcript-2 (EAT2), wherein the CAR comprises:
(i) an extracellular antigen binding domain;
(ii) a transmembrane domain; and
(iii) an intracellular signaling domain, and
wherein the CAR is linked to an intracellular domain derived from SAP via a peptide linker.

In certain embodiments, the CAR is linked to the intracellular domain derived from SAP or the intracellular domain derived from EAT2 via P2A, T2A, or E2A.

In certain embodiments, the polypeptide comprises an amino acid sequence at least 90%, at least 95%, or 100% identical to the amino acid sequence of SEQ ID NO:86 or SEQ ID NO:87.

In certain embodiments, the polypeptide comprises an amino acid sequence at least 90%, at least 95%, or 100% identical to the amino acid sequence of SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, or SEQ ID NO:145.

In yet another aspect, provided herein are polynucleotide. In certain embodiments, provided herein is a polynucleotide encoding the CAR provided herein or the polypeptide provided herein.

In yet another aspect, provided herein are vectors. In certain embodiments, provided herein is a vector comprising the polynucleotide provided herein.

In yet another aspect, provided herein are pharmaceutical compositions. In certain embodiments, provided herein is a pharmaceutical composition comprising (i) the CAR provided herein, the engineered immune cell provided herein, the polypeptide provided herein, the polynucleotide provided herein, or the vector provided herein; and (ii) a pharmaceutically acceptable excipient.

In yet another aspect, provided herein are methods of treating a disease or disorder in a subject. In certain embodiments, provided herein is a method of treating a disease or disorder in a subject, comprising administering to the subject an effective amount of the engineered immune cell provided herein or the pharmaceutical composition provided herein. In certain embodiments, the disease or disorder is cancer.

In yet another aspect, provided herein are methods of engineering an immune cell. In certain embodiments, provided herein is a method of engineering an immune cell, comprising introducing into the immune cell the polynucleotide provided herein, the vector provided herein, or the polypeptide provided herein.

### 4. BRIEF DESCRIPTION OF THE FIGURES

FIGS. 1A-1F illustrate the dysfunction of natural NKG2D in NKG2D-CAR-NK. FIG. 1A: CAR constructs of CAR0 and CAR2, and the expression of CARs on primary human NK cells. FIGS. 1B-1C: NKG2D and 2B4 expression on CAR0-NK and CAR2-NK cells. **P<0.01, ***P<0.001. FIG. 1D: CAR-NK cells killing assay, incubation after 10 hours. If there is CD19 on 293T, CAR2 killing is very high, but if no CD19, via NKG2D stimulation, CAR2 killing ability is significantly reduced, same thing happened in HUH7 cells. ***p<0.001. FIG. 1E: Duolink signals between CAR+DAP10, and NKG2D+DAP10 in CAR-NK cells stimulated by 293T-CD19-MICA. Left four images showed the Duolink PLA signal of the bright points, right four images showed the DAPI stained nuclear, cells with large nuclear were NK cells, and target cells were with smaller nuclear. FIG. 1F: The Duolink PLA signal number calculation as in FIG. 1E, ****P<0.0001.

FIGS. 2A-2E illustrate complete signaling 2B4-CAR mediated anti-tumor activity of NK cells. FIG. 2A: CAR construct structure designs, with ScFv of anti-CD19 or anti-GPC3. FIG. 2B: FACS analysis of phenotypes of 293T, 293T-CD19, NALM6 and HUH7. CD19 expressed in 293T-CD19, but not in 293T-WT (left), CD19 expressed on NALM6 cells (middle), and GPC3 expressed on HUH7 cells (right). FIG. 2C: CAR-NK92 cells kill target cells, 293T, 293T-CD19 (top, 4 hours), NALM6 (middle, 20 hours), HUH7 (bottom, 6 hours) at the indicated effector:target ratios. The mean percentage of specific target cell lysis±SEM is shown. FIGS. 2D and 2E: CD107a (FIG. 2D) and IFNg (FIG. 2E) expression was FACS analyzed in CAR-NK92 cells after stimulation by 293T, 293T-CD19 and NALM6. Data were standardized to the percentage of media treated CAR-NK92 cells, mean±SEM is shown. *P<0.05, ***p<0.001

FIGS. 3A-3C illustrate the SAP boosted complete signaling CAR. FIG. 3A: CAR constructs design with SAP. FIG. 3B: Signaling boosted NKG2D CAR and 2B4 CAR-NK cells killing assays 4 hours after incubation. FIG. 3C: IFNg production and degranulation of CAR-NK cells, stimulated with target cells as indicated. *P<0.05, **P<0.01, ***P<0.001.

FIGS. 4A-4D illustrate 2B4-DAP10-CD3Z CAR-NK have normal NKG2D function. FIG. 4A: CAR constructs design. FIG. 4B: NKG2D and 2B4 expression on CAR-NK cells. FIG. 4C: NKG2D and DAP10 interaction on NKG2D CAR-NK cells is reduced, but normal in 2B4 CAR-NK cells. ****P<0.0001, ns means no significance. FIG. 4D: killing assay of 10 h co-incubation of CAR-NK cells to kill target cells as indicated.

FIGS. 5A-5D illustrates that csCAR can overcome HLA-E barrier effectively. FIG. 5A: CAR constructs design. FIG. 5B: HLA-E expression on target cells of 293T-CD19, 293T-CD19-HLA-E, and NALM6 cells. FIG. 5C: NKG2A expression CAR-NK cells. FIG. 5D: CAR-NK killing assay to 293T-CD19, 293T-CD19-HLA-E after 4 hours incubation and NALM6 cells after 20 h incubation.

FIGS. 6A-H illustrates the domains of NK-CAR in NK activation and killing. FIGS. 6A and 6E: Vector constructs encoding the CD19 specific-CAR2, and -CAR4 with domain deficient of DAP10 or CD3Z, and different location of DAP10 (A), and changed location of DAP10 of CAR4, CAR5, CAR6, CAR8 to generate CAR4B, CAR5B, CAR6B, and CAR8B. FIG. 6B: NK cells were incubated with target cells of 293T or 293T-CD19 at the indicated effector:target ratio 4 hours after incubation. The mean percentage of specific target cell lysis±SEM is shown. FIGS. 6C and 6D: IFNg (FIG. 6C) and CD107a (FIG. 6D) expression was FACS analyzed in CAR-NK92 cells after stimulation by 293T, 293T-CD19 and NALM6. Data were standardized to the percentage of media treated CAR-NK92 cells, mean±SEM is shown. *P<0.05, **P<0.01, ***P<0.001. FIG. 6F: NK cells were incubated with target cells of NALM6 at the indicated effector:target ratio 20 hours after incubation. The mean percentage of specific target cell lysis±SEM is shown. FIGS. 6G and 6H: IFNg (FIG. 6H) and CD107a (FIG. 6G) expression was FACS analyzed in CAR-NK92 cells after stimulation by 293T, 293T-CD19 and NALM6. Data were standardized to the percentage of media treated CAR-NK92 cells, mean±SEM is shown. *P<0.05, **P<0.01.

FIGS. 7A-7E illustrates the wider screening of SLAMF and non-SLAMF csCARs in NK cell activation and killing. FIG. 7A: CAR constructs design. FIG. 7B: killing assay of CAR-NK cells to 293T-CD19, 293T-CD19-HLA-E after 4 hours incubation and NALM6 after 20 hours incubation. FIG. 7C: IFNg production and degranulation of CAR-NK cells stimulated by target cells. FIG. 7D: killing assay of CAR16B-NK cells to NALM6 20 hours incubation was better than that of CAR16-NK cells. FIG. 7E: IFNg production and degranulation of CAR16B-NK cells stimulated by target cells, *P<0.05, **P<0.01, ***P<0.001, ns means no significance.

FIG. 8 illustrates several SLMAF family genes that are expressed on NK cells, excluding SLAMF2 (CD48), which has no intracellular signaling domain.

### 5. DETAILED DESCRIPTION OF THE INVENTION

Provided herein are CARs, engineered immune cells (e.g., NK cells, NKT cells, and T cells), related polypeptides, related polynucleotides and vectors, related pharmaceutical compositions, related methods of treating a disease, and related methods of engineering an immune cells (e.g., NK cells, NKT cells, and T cells). In certain embodiments, provided herein is a CAR (see Section 5.2) comprising an extracellular antigen binding domain (see Section 5.2.2), an transmembrane domain (see Section 4.2.3), an intracellular signaling domain (see Section 5.2.4), optionally a hinge domain (see Section 5.2.5) and optionally one or more peptide linkers (see Section 5.2.6). In certain embodiments, provided herein is an engineered immune cell (see Section 5.3). In certain embodiments, provided herein is an engineered immune cell comprising the CAR as provided in Section 5.2. In certain embodiments, provided herein is an engineered immune cell comprising any CAR known in the art and an exogenously introduced polynucleotide encoding an intracellular domain derived from SAP or EAT2. In certain embodiments, provided herein is an engineered immune cell expressing any CAR known in the art and an intracellular domain derived from SAP or EAT2. In certain embodiments, provided herein is an engineered immune cell comprising a CAR that comprises an intracellular domain derived from SAP or EAT2. In certain embodiments, provided herein is a polypeptide (see Section 5.4). In certain embodiments, provided herein is a polypeptide comprising the CAR provided in Section 5.2. In certain embodiments, provided herein is a polypeptide comprising a CAR known in the art and an intracellular domain derived from SAP or EAT2 and the CAR is linked to the intracellular domain derived from SAP or EAT2 via a flexible linker. In certain embodiments, provided herein is a polynucleotide (see Section 5.5). In certain embodiments, provided herein is a vector (see Section 5.5). In certain embodiments, provided herein is a pharmaceutical composition comprising (i) the CAR provided in Section 5.2, the engineered immune cell provided in Section 5.3, the polypeptide provided in Section 5.4, the polynucleotide provided in Section 5.5, or the vector provided in Section 5.5; and (ii) a pharmaceutically acceptable excipient (see Section 5.6). In certain embodiments, provided herein is a therapeutic method or use related to the engineered immune cell (see Section 5.7). In certain embodiments, provided herein is a method of engineering an immune cell (see Section 5.8). In certain embodiments, provided herein are exemplary assays that can be used to make, use and measure the compositions provided herein (see Section 5.9).

In certain embodiments, an engineered immune cell provided herein is an NK cell, an NKT cell, or a T cell.

In certain embodiments, the CARs provided herein can enhance the killing activities of the engineered immune cells. In certain embodiments, the CARs provided herein can enhance the killing activities of the engineered NK cells.

In certain embodiments, the CAR provided herein comprises a SLAMF or non-SLAMF hinge/transmembrane domain/intracellular domain (ICD), the ICD of DAP10, and the CD3zeta signaling domain to mediate robust antigen-specific NK signaling. In certain embodiments, the CAR provided herein comprises DAP10 linked to the transmembrane domain directly or via a peptide linker. In certain embodiments, the CAR provided herein comprises signal booster polypeptide SAP or EAT2. In certain embodiments, the engineered immune cells, derived from human peripheral blood cells, significantly enhanced anti-tumor activities comparing with T-CAR or the reported NKG2D CARs, including 2B4 CAR, CD229 CAR, NTB-A CAR, CD84 CAR, CRACC CAR, CD28H CAR, CD2 CAR, DNAM1 CAR. In certain embodiments, without being bound by any particular theory, the CAR provided can efficiently block the HLA-E stimulated inhibitory signaling, thus overcoming the inhibitory receptor signaling barrier in the NK cell activation process, leading to more effective NK cell functions and further enhanced anti-tumor activities.

NKG2D CAR is a very efficient NK-CAR reported so far, which is composed of scFv-CD8 hinge-NKG2D TM-2B4 ICD-CD3zeta. The NKG2D CAR only used the transmembrane domain (TM) of NKG2D, which can recruit DAP10 to the CAR tail and play the role of co-stimulator and trigger signals as the natural NKG2D receptors (Li et al. (2018) Human iPSC-Derived Natural Killer Cells Engineered with Chimeric Antigen Receptors Enhance Anti-tumor Activity. Cell Stem Cell. 23(2):181-192.e5). However, without being bound by any particular theory, this may occupy the DAP10, and results in the deficient recruitment of DAP10 to the natural NKG2D, thus the function of natural NKG2D to be impaired. Without being bound by any particular theory, this phenomenon has been identified and confirmed in the studies related to the CAR provided herein. Without being bound by any particular theory, multifunctional NK cells can be activated by multiple activation receptors, and many tumor cells express NKG2D ligands, such as MICA/MICB and ULBP1-3, so keeping the complete function of NKG2D is very important for tumor cell clearance. Without being bound by any particular theory, we designed novel NK CARs that different from the NKG2D CAR.

Without being bound by any particular theory, to control NK cell responses, an integration of activation and inhibition balance is required. Without being bound by any particular theory, synergistic activation of activation receptor pairs can sufficiently trigger NK cell activation, such as 2B4 and NKG2D, or 2B4 and DNAM-1. Without being bound by any particular theory, this synergy is sufficient to overcome inhibitory signaling of inhibitory receptors via c-Cbl or SHP dependent inhibition. Without being bound by any particular theory, among the activation receptors, 1) 2B4 signaling is Fyn dependent and Syk-independent, which can phosphorylate the adaptor SLP-76 on Tyr113; 2) NKG2D recruits adaptor molecule DAP10 and DNAM-1 using the tail of an immunoreceptor tyrosine tail (ITT)-like motif to induce Syk-independent phosphorylation of SLP-76 on Tyr128; and 3) CD16 signaling via ITAM results in Syk-dependent phosphorylation of SLP-76 on both Tyr113 and Tyr128. Without being bound by any particular theory, for the full activation of NK cells, synergy provides sufficient and complete activation signals and overcomes the inhibitory receptor signaling barrier, which is the origin of the design concept of our new NK CARs (Long et al. (2013) Controlling natural killer cell responses: integration of signals for activation and inhibition. Annu Rev Immunol. 31:227-58).

Without being bound by any particular theory, the cytoplasmic tail of the SLAM family (SLAMF) molecules, such as 2B4, CD229, NTB-A, CD84 and CRACC, contains an immunoreceptor tyrosine-based switch motif (ITSM) for the signaling. Without being bound by any particular theory, the phosphorylated tyrosine of ITSM can bind to SHP, and SHP provides inhibitory signal for the NK cells. Without being bound by any particular theory, phosphorylated ITSMs can also recruit SLAM-associated protein (SAP) and Ewing's sarcoma-activated transcript-2 (EAT-2) (Ma et al. (2007) Regulation of cellular and humoral immune responses by the SLAM and SAP families of molecules. Annu Rev Immunol. 25:337-79; Claus et al. (2008) Regulation of NK cell activity by 2B4, NTB-A and CRACC. Front Biosci. 13:956-65). Without being bound by any particular theory, ITSM-bound SAP and EAT2 molecule can recruit the Src-family kinase Fyn, and then phosphorylate Phospholipase C-γ or Vav-1 (Chen et al. (2004) Molecular dissection of 2B4 signaling: implications for signal transduction by SLAM-related receptors. Mol Cell Biol. 24(12):5144-56; Eissmann et al. (2006) Molecular analysis of NTB-A signaling: a role for EAT-2 in NTB-A-mediated activation of human NK cells. J Immunol. 177(5):3170-7). Without being bound by any particular theory, the relative amount of SAP and SLAMF receptor decides the direction of effector cells; insufficient SAP results in predominantly inhibitory signals, and increased ligand density or SLAMF receptor expression can make SAP expression limiting and result in inhibition of effector cell function (Waggoner et al. (2012) Evolving role of 2B4/CD244 in T and NK cell responses during virus infection. Front Immunol. 3:377). Without being bound by any particular theory, sufficient SAP can competitively bind the pTyr of ITSM at the SLAMF tail, and then prevent ITSM binding to the inhibitory signal SHP, so that Fyn molecule can be recruited and lead to the SLP-76 activation via phosphorylation at Tyr113.

### 5.1 Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dubel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies), as described below. An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

“Single domain antibody” or “sdAb” as used herein refers to a single monomeric variable antibody domain and which is capable of antigen binding (e.g., single domain antibodies that bind to CD19). Single domain antibodies include VHH domains as described herein. Examples of single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. For example, a single domain antibody can be derived from antibodies raised in Camelidae species, for example in camel, llama, dromedary, alpaca and guanaco, as described herein. Other species besides Camelidae may produce heavy chain antibodies naturally devoid of light chain; VHHs derived from such other species are within the scope of the disclosure. In some embodiments, the single domain antibody (e.g., VHH) provided herein has a structure of FR1-CDR1-FR2-CDR2-FR3-CDR3-FR4. Single domain antibodies may be genetically fused or chemically conjugated to another molecule (e.g., an agent) as described herein. Single domain antibodies may be part of a bigger binding molecule (e.g., a multispecific antibody or a chimeric antigen receptor).

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as radioimmunoassay (RIA) and enzyme linked immunosorbent assay (ELISA). Typically, a specific or selective reaction is at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by fluorescence activated cell sorting (FACS) analysis or RIA. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (K_{D}) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

In certain embodiments, the binding molecules or antigen binding domains can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody comprise at least a portion of an immunoglobulin constant region (Fe), typically that of a human immunoglobulin. For further details, see, Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos. 6,800,738; 6,719,971; 6,639,055; 6,407,213; and 6,054,297.

In certain embodiments, the binding molecules or antigen binding domains can comprise portions of a “fully human antibody” or “human antibody,” wherein the terms are used interchangeably herein and refer to an antibody that comprises a human variable region and, for example, a human constant region. The binding molecules may comprise a single domain antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1: 755-68 (2006)). Also available for the preparation of human monoclonal antibodies are methods described in Cole et al., Monoclonal Antibodies and Cancer Therapy 77 (1985); Boerner et al., J. Immunol. 147(1):86-95 (1991); and van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6(5):561-66 (1995); Bruggemann and Taussing, Curr. Opin. Biotechnol. 8(4):455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, the binding molecules or antigen binding domains can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the binding molecules or antigen binding domains can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid iomerizatio or deamidation, methionine oxidation or asparagine or glutamine deamidation, each monoclonal antibody typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while p and E contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH R-sheet framework, or one of three hypervariable regions (L1, L2 or L3) within the non-framework region of the antibody VL β-sheet framework. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dubel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27(1):55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Pluckthun, J. Mol. Biol. 309: 657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in Table 1 below.

TABLE 1
Exemplary CDRs According to Various Numbering Systems

| Loop | Kabat | AbM | Chothia | Contact | IMGT |
|---|---|---|---|---|---|
| CDR L1 | L24--L34 | L24--L34 | L26--L32 or | L30--L36 | L27--L38 |
| | | | L24--L34 | | |
| CDR L2 | L50--L56 | L50--L56 | L50--L52 or | L46--L55 | L56--L65 |
| | | | L50--L56 | | |
| CDR L3 | L89--L97 | L89--L97 | L91--L96 or | L89--L96 | L105-L117 |
| | | | L89--L97 | | |
| CDR H1 | H31--H35B | H26--H35B | H26--H32 . . . 34 | H30--H35B | H27--H38 |
| | (Kabat Numbering) | | | | |
| CDR H1 | H31--H35 | H26--H35 | H26--H32 | H30--H35 | |
| | (Chothia Numbering) | | | | |
| CDR H2 | H50--H65 | H50--H58 | H53--H55 or | H47--H58 | H56--H65 |
| | | | H52--H56 | | |
| CDR H3 | H95--H102 | H95--H102 | H96--H101 or | H93--H101 | H105-H117 |
| | | | H95--H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR as set forth in a specific VH or VHH” includes any CDR1 as defined by the exemplary CDR numbering systems described above, but is not limited thereby. Once a variable region (e.g., a VHH, VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies (e.g., single domain antibodies), diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody comprising a single domain antibody sequence) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

A “blocking” antibody or an “antagonist” antibody is one that inhibits or reduces a biological activity of the antigen it binds. In some embodiments, blocking antibodies or antagonist antibodies substantially or completely inhibit the biological activity of the antigen.

An “agonist” or activating antibody is one that enhances or initiates signaling by the antigen to which it binds. In some embodiments, agonist antibodies cause or activate signaling without the presence of the natural ligand.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

“Chimeric antigen receptor” or “CAR” as used herein refers to genetically engineered receptors, which can be used to graft one or more antigen specificity onto immune effector cells, such as NK cells, NKT cells, and T cells. Some CARs are also known as “artificial T-cell receptors,” “chimeric T cell receptors,” or “chimeric immune receptors.” In some embodiments, the CAR comprises an extracellular antigen binding domain specific for one or more antigens (such as tumor antigens), a transmembrane domain, and an intracellular signaling domain of a T cell, NKT cell, or NK cell and/or other receptors. “CAR-T cell” refers to a T cell that expresses a CAR. “CAR-NKT cell” refers to an NK cell that expresses a CAR. “CAR-NK cell” refers to an NK cell that expresses a CAR.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding a single domain antibody or an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding a CAR or an sdAb described herein is a nucleic acid molecule that is identified and separated from at least one contaminant nucleic acid molecule with which it is ordinarily associated in the environment in which it was produced.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “autologous” is meant to refer to any material derived from the same individual to whom it is later to be re-introduced into the individual.

“Allogeneic” refers to a graft derived from a different individual of the same species.

The term “transfected” or “transformed” or “transduced” as used herein refers to a process by which exogenous polynucleotide is transferred or introduced into the host cell. A “transfected” or “transformed” or “transduced” cell is one which has been transfected, transformed or transduced with exogenous polynucleotide. The cell includes the primary subject cell and its progeny. In certain embodiments, an exogenously introduced polynucleotide is not a polynucleotide that exits naturally in a cell, e.g., an engineered immune cell.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™ polyethylene glycol (PEG), and PLURONICS™. Other examples of pharmaceutically acceptable excipients are described in Remington and Gennaro, Remington's Pharmaceutical Sciences (18th ed. 1990).

As used herein, the term “pharmaceutically acceptable” when used in reference to a carrier, is intended to mean that the carrier, diluent or excipient is not toxic or otherwise undesirable, (i.e., the material may be administered to a subject without causing any undesirable biological effects), and it is compatible with the other ingredients of the formulation. The term “carrier” refers to a diluent, adjuvant, excipient, or vehicle with which the therapeutic is administered. Such pharmaceutical carriers can be sterile liquids, such as saline solutions. A saline solution can be a carrier when the pharmaceutical composition is administered intravenously. Saline solutions and aqueous dextrose and glycerol solutions can also be employed as liquid carriers, particularly for injectable solutions. The composition, if desired, can also contain minor amounts of wetting or emulsifying agents, or pH buffering agents.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain a binding molecule (e.g., an antibody), for example, in isolated or purified form, together with a suitable amount of excipients.

The term “effective amount” or “therapeutically effective amount” as used herein refers to the amount of a single domain antibody or a therapeutic molecule comprising an agent and the single domain antibody or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., diabetes or a cancer).

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As well-known in the art, polypeptide sequences and fragments thereof can be used in reversed format except for the signal peptide (SP). Various polypeptide sequences as provided herein, such as the amino acid sequences of hinge domain, transmembrane domain or intracellular domain of a polypeptide, can be used in reversed format. In other words, it is understood that whenever an amino acid sequence is provided herein, the reversed version of such is also provided in the application. As an illustration for clarity, the amino acid sequence of DAP10 intracellular domain (ICD) is LCARPRRSPAQEDGKVYINMPGRG (SEQ ID NO:20), whereas a reversed version of the amino acid sequence of DAP10 intracellular domain (ICD) is GRGPMNIYVKGDEDQAPSRRPRACL (SEQ ID NO:177). Similarly, the amino acid sequence of SAP is SEQ ID NO:86, whereas the reversed version of such is SEQ ID NO:178. The amino acid sequence of EAT2 is SEQ ID NO:87, whereas the reversed version of such is SEQ ID NO:179.

### 5.2 Chimeric Antigen Receptor (CARs)

In one aspect, provided herein are chimeric antigen receptors (CARs). In certain embodiments, provided herein is a chimeric antigen receptor (CAR), comprising:
(i) an extracellular antigen binding domain;
(ii) a transmembrane domain; and
(iii) an intracellular signaling domain comprising an intracellular domain derived from a first polypeptide,
wherein the intracellular domain derived from the first polypeptide is linked to the C-terminus of the transmembrane domain directly or via a peptide linker, and wherein the first polypeptide is DAP10 or DAP12.

In certain embodiments, provided herein is a chimeric antigen receptor (CAR), comprising:
(i) an extracellular antigen binding domain;
(ii) a transmembrane domain comprising a transmembrane domain derived from a second polypeptide; and
(iii) an intracellular signaling domain comprising an intracellular domain derived from a first polypeptide,
wherein the second polypeptide is different from the first polypeptide, wherein the intracellular domain derived from the first polypeptide is linked to the C-terminus of the transmembrane domain derived from the second polypeptide directly or via a peptide linker, and wherein the first polypeptide is DNAM1.

In certain embodiments, the transmembrane domain of the CAR comprises a transmembrane domain derived from a second polypeptide, wherein the second polypeptide is different from the first polypeptide, and wherein the intracellular domain derived from the first polypeptide is linked to the C-terminus of the transmembrane domain derived from the second polypeptide directly or via a peptide linker.

In certain embodiments, the intracellular signaling domain of the CAR further comprises a intracellular domain derived from a third polypeptide, wherein the third polypeptide is different from the first polypeptide.

In certain embodiments, the second polypeptide and the third polypeptide is the same polypeptide.

In certain embodiments, the CAR further comprises a hinge domain between the C-terminus of the extracellular antigen binding domain and the N-terminus of the transmembrane domain.

In certain embodiments, the hinge domain of the CAR comprises a hinge domain derived from the second polypeptide.

In certain embodiments, the second polypeptide or the third polypeptide is a signaling lymphocytic activation molecule family (SLAMF) receptor, CD2, CD28H, DNAM1, CD28, 4-1BB, OX40, KIR-S, NKG2C, or NKG2E. In certain embodiments, the SLAMF receptor is SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), or SLAMF9 (CD84H1, SF2001). In certain embodiments, the KIR-S is KIR2DS1, KIR2DS2, KIR2DS3, KIR2DS4, KIR2DS5 or KIR3DS1.

In certain embodiments, the intracellular signaling domain of the CAR further comprises an intracellular domain derived from CD3zeta.

In certain embodiments, the intracellular signaling domain of the CAR further comprises an intracellular domain derived from SLAM-associated protein (SAP). In certain embodiments, the intracellular signaling domain of the CAR further comprises an intracellular domain derived from EAT2.

In certain embodiments, the intracellular domain derived from CD3zeta is linked to the intracellular domain derived from SAP or the intracellular domain derived from EAT2 directly or via a peptide linker. In certain embodiments, the intracellular domain derived from CD3zeta is linked to the intracellular domain derived from SAP or the intracellular domain derived from EAT2 via P2A, T2A, or E2A.

In certain embodiments, the extracellular antigen binding domain of the CAR comprises an scFv. In certain embodiments, the extracellular antigen binding domain of the CAR binds to a tumor antigen or a fragment thereof.

In certain embodiments, the first polypeptide is DAP10.

In certain embodiments, the CAR comprises an amino acid sequence at least 90%, at least 95%, or 100% identical to the amino acid sequence of SEQ ID NO:20.

In certain embodiments, the CAR comprises an amino acid sequence at least 90%, at least 95%, or 100% identical to the amino acid sequence of SEQ ID NO:86.

In certain embodiments, the CAR comprises an amino acid sequence at least 90%, at least 95%, or 100% identical to the amino acid sequence of SEQ ID NO:87.

In certain embodiments, the CAR comprises an amino acid sequence at least 90%, at least 95%, or 100% identical to the amino acid sequence of SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, or SEQ ID NO:145.

In certain embodiments, the CAR, from N-terminus to C-terminus, comprises
(i) an extracellular antigen binding domain;
(ii) a transmembrane domain comprising an transmembrane domain derived from the second polypeptide;
(iii) an intracellular signaling domain comprising
   (iii)-(a) an intracellular domain derived from DAP10;
   (iii)-(b) an intracellular domain derived from CD3zeta; and
   (iii)-(c) an intracellular domain derived from SAP or an intracellular domain derived from EAT2, wherein the intracellular domains within the intracellular signaling domain are linked to each other directly or via one or more peptide linkers, and wherein the extracellular antigen binding domain, the transmembrane domain, and the intracellular domain are linked to each other directly or via one or more peptide linkers.

### 5.2.1 Exemplary CAR

In certain embodiments, the CAR provided herein, from N-terminus to C-terminus, comprises:
(i) an extracellular antigen binding domain;
(ii) optionally a hinge domain that can be, but is not limited to, a hinge domain derived from a second polypeptide;
(iii) a transmembrane domain comprising an transmembrane domain derived from a second polypeptide;
(iv) optionally a peptide linker that can be, but is not limited to, a peptide linker provided herein;
(v) an intracellular signaling domain comprising:
   (v)-(a) an intracellular domain derived from a first polypeptide that is DAP10, DNAM1, or DAP12;
   (v)-(b) optionally an intracellular domain derived from a third polypeptide;
   (v)-(c) optionally an intracellular domain derived from CD3zeta;
   (v)-(d) optionally a peptide linker, that can be, but is not limited to, P2A, T2A, or E2A;
   (v)-(e) optionally an intracellular domain derived from SAP or an intracellular domain derived from EAT2.

In certain embodiments, the second polypeptide is different from the first polypeptide. In certain embodiments, the third polypeptide is different from the first polypeptide. In certain embodiments, the second polypeptide and the third polypeptide are the same polypeptide. In certain embodiments, the second polypeptide and the third polypeptide are different polypeptides. In certain embodiments, the second polypeptide or the third polypeptide is a signaling lymphocytic activation molecule family (SLAMF) receptor, CD2, CD28H, DNAM1, CD28, 4-1BB, OX40, KIR-S, NKG2C, or NKG2E. In certain embodiments, the SLAMF receptor is SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), or SLAMF9 (CD84H1, SF2001). In certain embodiments, the KIR-S is KIR2DS1, KIR2DS2, KIR2DS3, KIR2DS4, KIR2DS5 or KIR3DS1.

In certain embodiments, the CAR comprises the amino acid sequence of SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, or SEQ ID NO:145.

In certain embodiments, the CAR provided herein comprises one or more elements as shown in Table 2. In certain embodiments, the CAR provided herein comprises the elements as illustrated in Table 2 while having one or more peptide linkers between certain elements, for example, between the hinge domain and the transmembrane domain, between the transmembrane domain and the intracellular signaling domain, and between various intracellular domains derived from different polypeptides within the intracellular signaling domain. In certain embodiments, the symbol “-” in Table 2 represents linking directly or via a peptide linker, for example via a peptide linker as provided in Section 5.2.6.

TABLE 2
Exemplary CAR constructs

| Construct | Elements |
|---|---|
| CAR1 | 2B4 hinge-2B4 TM-2B4 ICD |
| CAR2 | CD8 hinge-NKG2D TM-2B4 ICD-CD3zeta |
| CAR3 | CD8 hinge-NKG2D TM-2B4 ICD-CD3zeta-P2A-SAP |
| CAR4 | ScFv-CD8 hinge-2B4 TM-2B4 ICD-DAP10-CD3zeta |
| CAR4A | CD8 hinge-2B4 TM-2B4 ICD-CD3zeta |
| CAR4B | CD8 hinge-2B4 TM-DAP10-2B4 ICD-CD3zeta |
| CAR4C | CD8 hinge-2B4 TM-2B4 ICD-DAP10 |
| CAR5 | CD8 hinge-2B4 TM-2B4 ICD-DAP10-CD3zeta-P2A-SAP |
| CAR5B | CD8 hinge-2B4 TM-DAP10-2B4 ICD-CD3zeta-P2A-SAP |
| CAR6 | 2B4 hinge-2B4 TM-2B4 ICD-DAP10-CD3zeta |
| CAR6B | 2B4 hinge-2B4 TM-DAP10-2B4 ICD-CD3zeta |
| CAR8 | 2B4 hinge-2B4 TM-2B4 ICD-DAP10-CD3zeta-P2A-SAP |
| CAR8B | 2B4 hinge-2B4 TM-DAP10-2B4 ICD-CD3zeta-P2A-SAP |
| CAR10 | 2B4 hinge-2B4 TM-2B4 ICD-DAP10-CD3zeta-P2A-EAT2 |
| CAR11 | CD229 hinge-CD229 TM-DAP10-CD229 ICD-CD3zeta |
| CAR12 | CD229 hinge-CD229 TM-DAP10-CD229 ICD-CD3zeta-P2A-SAP |
| CAR13 | NTB-A hinge-NTB-A TM-DAP10-NTB-A ICD-CD3zeta |
| CAR14 | NTB-A hinge-NTB-A TM-DAP10-NTB-A ICD-CD3zeta-P2A-SAP |
| CAR15 | CRACC hinge-CRACC TM-DAP10-CRACC ICD-CD3zeta |
| CAR16 | CRACC hinge-CRACC TM-DAP10-CRACC ICD-CD3zeta-P2A-SAP |
| CAR16B | CRACC hinge-CRACC TM-DAP10-CRACC ICD-CD3zeta-P2A-EAT2 |
| CAR17 | CD84 hinge-CD84 TM-DAP10-CD84 ICD-CD3zeta |
| CAR18 | CD84 hinge-CD84 TM-DAP10-CD84 ICD-CD3zeta-P2A-SAP |
| CAR19 | CD2 hinge-CD2 TM-DAP10-CD2 ICD-CD3zeta |
| CAR20 | CD2 hinge-CD2 TM-DAP10-CD2 ICD-CD3zeta-P2A-SAP |
| CAR21 | CD28H hinge-CD28H TM-DAP10-CD28H ICD-CD3zeta |
| CAR22 | CD28H hinge-CD28H TM-DAP10-CD28H ICD-CD3zeta-P2A-SAP |
| CAR23 | DNAM1 hinge-DNAM1 TM-DAP10-DNAMI ICD-CD3zeta |
| CAR24 | DNAM1 hinge-DNAM1 TM-DAP10-DNAM1 ICD-CD3zeta-P2A-SAP |
| CAR25 | CD28 hinge-CD28 TM-DAP10 ICD-CD28 ICD-CD3zeta |
| CAR26 | CD28 hinge-CD28 TM-DAP10 ICD-CD28 ICD-CD3zeta-P2A-SAP |
| CAR27 | 4-1BB hinge-41BB TM-DAP10 ICD-4-1BB ICD-CD3zeta |
| CAR28 | 4-1BB hinge-41BB TM-DAP10 ICD-4-1BB ICD-CD3zeta-P2A-SAP |
| CAR29 | OX40 hinge-OX40 TM-DAP10 ICD-OX40 ICD-CD3zeta |
| CAR30 | OX40 hinge-OX40 TM-DAP10 ICD-OX40 ICD-CD3zeta-P2A-SAP |
| CAR31 | KIR2DS1 hinge-KIR2DS1 TM-DAP10 ICD-CD3zeta |
| CAR32 | KIR2DS1 hinge-KIR2DS1 TM-DAP10 ICD-CD3zeta-P2A-SAP |
| CAR33 | KIR2DS2 hinge-KIR2DS2 TM-DAP10 ICD-CD3zeta |
| CAR34 | KIR2DS2 hinge-KIR2DS2 TM-DAP10 ICD-CD3zeta-P2A-SAP |
| CAR35 | KIR2DS3 hinge-KIR2DS3 TM-DAP10 ICD-CD3zeta |
| CAR36 | KIR2DS3 hinge-KIR2DS3 TM-DAP10 ICD-CD3zeta-P2A-SAP |
| CAR37 | KIR2DS4 hinge-KIR2DS4 TM-DAP10 ICD-CD3zeta |
| CAR38 | KIR2DS4 hinge-KIR2DS4 TM-DAP10 ICD-CD3zeta-P2A-SAP |
| CAR39 | KIR2DS5 hinge-KIR2DS5 TM-DAP10 ICD-CD3zeta |
| CAR40 | KIR2DS5 hinge-KIR2DS5 TM-DAP10 ICD-CD3zeta-P2A-SAP |
| CAR41 | NKG2C hinge-NKG2C TM-DAP10 ICD-CD3zeta |
| CAR42 | NKG2C hinge-NKG2C TM-DAP10 ICD-CD3zeta-P2A-SAP |
| CAR43 | NKG2E hinge-NKG2E TM-DAP10 ICD-CD3zeta |
| CAR44 | NKG2E hinge-NKG2E TM-DAP10 ICD-CD3zeta-P2A-SAP |
| CAR45 | CD8 hinge-NKG2D TM-2B4 ICD-CD3zeta-SAP |
| CAR46 | CD8 hinge-2B4 TM-2B4 ICD-DAP10-CD3zeta-SAP |
| CAR47 | CD8 hinge-2B4 TM-DAP10-2B4 ICD-CD3zeta-SAP |
| CAR48 | 2B4 hinge-2B4 TM-2B4 ICD-DAP10-CD3zeta-SAP |
| CAR49 | 2B4 hinge-2B4 TM-DAP10-2B4 ICD-CD3zeta-SAP |
| CAR50 | 2B4 hinge-2B4 TM-2B4 ICD-DAP10-CD3zeta-EAT2 |
| CAR51 | CD229 hinge-CD229 TM-DAP10-CD229 ICD-CD3zeta-SAP |
| CAR52 | NTB-A hinge-NTB-A TM-DAP10-NTB-A ICD-CD3zeta-SAP |
| CAR53 | CRACC hinge-CRACC TM-DAP10-CRACC ICD-CD3zeta-SAP |
| CAR54 | CRACC hinge-CRACC TM-DAP10-CRACC ICD-CD3zeta-EAT2 |
| CAR55 | CD84 hinge-CD84 TM-DAP10-CD84 ICD-CD3zeta-SAP |
| CAR56 | CD2 hinge-CD2 TM-DAP10-CD2 ICD-CD3zeta-SAP |
| CAR57 | CD28H hinge-CD28H TM-DAP10-CD28H ICD-CD3zeta-SAP |
| CAR58 | DNAM1 hinge-DNAM1 TM-DAP10-DNAMI ICD-CD3zeta-SAP |
| CAR59 | CD28 hinge-CD28 TM-DAP10 ICD-CD28 ICD-CD3zeta-SAP |
| CAR60 | 4-1BB hinge-41BB TM-DAP10 ICD-4-1BB ICD-CD3zeta-SAP |
| CAR61 | OX40 hinge-OX40 TM-DAP10 ICD-OX40 ICD-CD3zeta-SAP |
| CAR62 | KIR2DS1 hinge-KIR2DS1 TM-DAP10 ICD-CD3zeta-SAP |
| CAR63 | KIR2DS2 hinge-KIR2DS2 TM-DAP10 ICD-CD3zeta-SAP |
| CAR64 | KIR2DS3 hinge-KIR2DS3 TM-DAP10 ICD-CD3zeta-SAP |
| CAR65 | KIR2DS4 hinge-KIR2DS4 TM-DAP10 ICD-CD3zeta-SAP |
| CAR66 | KIR2DS5 hinge-KIR2DS5 TM-DAP10 ICD-CD3zeta-SAP |
| CAR67 | NKG2C hinge-NKG2C TM-DAP10 ICD-CD3zeta-SAP |
| CAR68 | NKG2E hinge-NKG2E TM-DAP10 ICD-CD3zeta-SAP |

### 5.2.2 Extracellular Antigen Binding Domain

In certain embodiments, the extracellular antigen binding domain of the CAR provided herein recognizes and binds to a specific binding element on the target of interest. Non-limiting examples of the extracellular antigen binding domain include a single-chain variable fragment (scFv), nanobodies, ligands to cognate receptors, native receptors against targets, and small peptides. In certain embodiment, the extracellular antigen binding domain of the CAR comprises an scFv. In certain embodiments, the extracellular antigen binding domain of the CAR binds to a tumor antigen or a fragment thereof.

In certain embodiments, the extracellular antigen binding domain of the CAR binds to GPC3 or a fragment thereof. In certain embodiments, the extracellular antigen binding domain of the CAR binds to a polypeptide comprising the amino acid sequence of SEQ ID NO:92. In certain embodiments, the extracellular antigen binding domain of the CAR comprises an amino acid sequence at least 75%, 80%, 85%, 90%, 95%, 96%, 97%, 98%, 99%, or more identical to the amino acid sequence of SEQ ID NO: 170. In certain embodiments, the extracellular antigen binding domain of the CAR comprises the amino acid sequence of SEQ ID NO: 170. In certain embodiments, the extracellular antigen binding domain of the CAR binds to GPC3 and comprises the VH CDR1-3 and VL CDR1-3 in an scFv comprising an amino acid sequence at least 75%, 80%, 85%, 90%, 95%, 96%, 97%, 98%, 99%, or more identical to the amino acid sequence of SEQ ID NO:170. In certain embodiments, the extracellular antigen binding domain of the CAR binds to GPC3 and comprises the VH CDR1-3 and VL CDR1-3 in an scFv comprising the amino acid sequence of SEQ ID NO:170. In certain embodiments, the extracellular antigen binding domain of the CAR binds to GPC3 and comprises a VH CDR1 comprising the amino acid sequence of SEQ ID NO:171, a VH CDR2 comprising the amino acid sequence of SEQ ID NO:172, a VH CDR3 comprising the amino acid sequence of SEQ ID NO:173, a VL CDR1 comprising the amino acid sequence of SEQ ID NO:174, a VL CDR2 comprising the amino acid sequence of SEQ ID NO:175, and a VL CDR1 comprising the amino acid sequence of SEQ ID NO:176.

In certain embodiments, the extracellular antigen binding domain of the CAR binds to CD19 or a fragment thereof. In certain embodiments, the extracellular antigen binding domain of the CAR binds to a polypeptide comprising the amino acid sequence of SEQ ID NO:93. In certain embodiments, the extracellular antigen binding domain of the CAR comprises an amino acid sequence at least 75%, 80%, 85%, 90%, 95%, 96%, 97%, 98%, 99%, or more identical to the amino acid sequence of SEQ ID NO:163. In certain embodiments, the extracellular antigen binding domain of the CAR comprises the amino acid sequence of SEQ ID NO:163. In certain embodiments, the extracellular antigen binding domain of the CAR binds to CD19 and comprises the VH CDR1-3 and VL CDR1-3 in an scFv comprising an amino acid sequence at least 75%, 80%, 85%, 90%, 95%, 96%, 97%, 98%, 99%, or more identical to the amino acid sequence of SEQ ID NO:163. In certain embodiments, the extracellular antigen binding domain of the CAR binds to CD19 and comprises the VH CDR1-3 and VL CDR1-3 in an scFv comprising the amino acid sequence of SEQ ID NO:163. In certain embodiments, the extracellular antigen binding domain of the CAR binds to CD19 and comprises a VH CDR1 comprising the amino acid sequence of SEQ ID NO:164, a VH CDR2 comprising the amino acid sequence of SEQ ID NO:165, a VH CDR3 comprising the amino acid sequence of SEQ ID NO:166, a VL CDR1 comprising the amino acid sequence of SEQ ID NO:167, a VL CDR2 comprising the amino acid sequence of SEQ ID NO:168, and a VL CDR1 comprising the amino acid sequence of SEQ ID NO:169.

Various antigens suitable for targeting with a CAR are known in the art, and each are suitable for use with the present application. Tumor antigens are proteins that are produced by tumor cells that can elicit an immune response, particularly T-cell mediated immune responses. Exemplary tumor antigens include, but not limited to, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. In addition to CD19, B-cell differentiation antigens such as CD20 and CD37 are other candidates for target antigens in B-cell lymphoma.

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include: differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA and alpha-fetoprotein (AFP); overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA, human immunodeficiency viruses (HIV) antigen CD32a, hepatitis B virus (HBV) antigen HBsAg, and the human papillomavirus (HPV) antigens E6 and E7.

Other large, protein-based antigens include TSP-180, MAGE-4, MAGE-5, MAGE-6, RAGE, NY-ESO, p185erbB2, pl80erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, CLDN18.1, CLDN18.2, CLDN18.6, FAP, and TPS. In some more specific embodiments, the one or more additional antigen(s) is selected from a group consisting of CD20, CD22, CD33, CD38, BCMA, CS1, ROR1, GPC3, CD123, IL-13R, CD138, c-Met, EGFRvIII, GD-2, NY-ESO-1, MAGE A3, and glycolipid F77.

### 5.2.3 Transmembrane Domain

In certain embodiments, the CARs provided herein comprise a transmembrane domain that is linked to the extracellular antigen binding domain provided herein directly or via a hinge domain provided herein.

In certain embodiments, the transmembrane domain of the CAR comprises a transmembrane domain derived from a second polypeptide, wherein the second polypeptide is different from the first polypeptide, and wherein the intracellular domain derived from the first polypeptide is linked to the C-terminus of the transmembrane domain derived from the second polypeptide directly or via a peptide linker.

In certain embodiments, the second polypeptide is a signaling lymphocytic activation molecule family (SLAMF) receptor, CD2, CD28H, DNAM1, CD28, 4-1BB, OX40, KIR-S, NKG2C, or NKG2E. In certain embodiments, the third polypeptide is a signaling lymphocytic activation molecule family (SLAMF) receptor, CD2, CD28H, DNAM1, CD28, 4-1BB, OX40, KIR-S, NKG2C, or NKG2E. In certain embodiments, the SLAMF receptor is SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), or SLAMF9 (CD84H1, SF2001). In certain embodiments, the KIR-S is KIR2DS1, KIR2DS2, KIR2DS3, KIR2DS4, KIR2DS5 or KIR3DS1.

In certain embodiments, the transmembrane domain of the CAR comprises a transmembrane domain derived from SLAMF4 (CD244, 2B4). In certain embodiments, the transmembrane domain of the CAR comprises a transmembrane domain derived from SLAMF3 (CD229, Ly9). In certain embodiments, the transmembrane domain of the CAR comprises a transmembrane domain derived from SLAMF6 (CD352, NTB-A, SF2000). In certain embodiments, the transmembrane domain of the CAR comprises a transmembrane domain derived from SLAMF7 (CD319, CRACC, CS1). In certain embodiments, the transmembrane domain of the CAR comprises a transmembrane domain derived from SLAMF1 (CD150). In certain embodiments, the transmembrane domain of the CAR comprises a transmembrane domain derived from SLAMF2 (CD48, FimH). In certain embodiments, the transmembrane domain of the CAR comprises a transmembrane domain derived from SLAMF8 (CD353). In certain embodiments, the transmembrane domain of the CAR comprises a transmembrane domain derived from SLAMF9 (CD84H1, SF2001). In certain embodiments, the transmembrane domain of the CAR comprises a transmembrane domain derived from SLAMF5 (CD84). In certain embodiments, the transmembrane domain of the CAR comprises a transmembrane domain derived from NKG2D. In certain embodiments, the transmembrane domain of the CAR comprises a transmembrane domain derived from CD28H. In certain embodiments, the transmembrane domain of the CAR comprises a transmembrane domain derived from DNAM1. In certain embodiments, the transmembrane domain of the CAR comprises a transmembrane domain derived from CD2.

In some embodiments, the transmembrane domain of the CAR comprises the transmembrane domain (TD) of SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), SLAMF9 (CD84H1, SF2001), CD8, CD28, 4-1BB, OX40, 2B4, CD2, CD28H, DNAM1, KIR2DS1, KIR2DS2, KIR2DS3, KIR2DS4, KIR2DS5, KIR3DS1, NKG2C, or NKG2E. In certain embodiments, the transmembrane domain of the CAR comprises an amino acid sequence at least 75%, 80%, 85%, 90%, 95%, 96%, 97%, 98%, 99%, or more identical to the amino acid sequence of SEQ ID NO:8, SEQ ID NO:12, SEQ ID NO:16, SEQ ID NO:18, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:68, SEQ ID NO:72, SEQ ID NO:76, SEQ ID NO:80, or SEQ ID NO:84. In certain embodiments, the transmembrane domain of the CAR comprises the amino acid sequence of SEQ ID NO:8, SEQ ID NO:12, SEQ ID NO:16, SEQ ID NO:18, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:68, SEQ ID NO:72, SEQ ID NO:76, SEQ ID NO:80, or SEQ ID NO:84.

The transmembrane domain may be derived either from a natural or from a synthetic source. As used herein, a “transmembrane domain” refers to any protein structure that is thermodynamically stable in a cell membrane, preferably an eukaryotic cell membrane. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the CAR described herein is derived from a Type I single-pass membrane protein. In some embodiments, transmembrane domains from multi-pass membrane proteins may also be compatible for use in the CARs described herein. Multi-pass membrane proteins may comprise a complex (at least 2, 3, 4, 5, 6, 7 or more) alpha helices or a beta sheet structure. In some embodiments, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side.

In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRFl), CD160, CD19, IL-2R beta, IL-2R gamma, IL-7R a, ITGA1, VLA1, CD49a, ITGA4, IA4, CD49D, ITGA6, VLA-6, CD49f, ITGAD, CDl ld, ITGAE, CD103, ITGAL, CDl la, LFA-1, ITGAM, CDl lb, ITGAX, CDl lc, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C, whereas the intracellular domain of the CAR comprises an intracellular domain derived from a first polypeptide that is different from the peptides listed in this paragraph.

Transmembrane domains for use in the CARs described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776, the relevant disclosures of which are incorporated by reference herein.

### 5.2.4 Intracellular Signaling Domain

In certain embodiments, the intracellular signaling domain of the CAR provided herein comprises an intracellular domain derived from a first polypeptide that is DAP10, DNAM1 or DAP12. In certain embodiments, the intracellular signaling domain of the CAR provided herein comprises the intracellular domain of DAP10, DNAM1 or DAP12. In certain embodiments, the first polypeptide is DAP10. In certain embodiments, the intracellular signaling domain of the CAR provided herein comprises an intracellular domain derived from DAP10. In certain embodiments, the intracellular signaling domain of the CAR provided herein comprises the intracellular domain of DAP10 (DAP10 ICD). In certain embodiments, the intracellular domain derived from the first polypeptide is linked to the C-terminus of the transmembrane domain directly or via a peptide linker provided herein.

In certain embodiments, the first polypeptide is capable of activating PI3K at the site of membrane. In certain embodiments, the intracellular domain derived from the first polypeptide (i.e., DAP10, DNAM1 or DAP12) is linked to the C-terminus of the transmembrane domain via a peptide linker. In certain embodiments, the peptide linker between the intracellular domain derived from the first polypeptide and the C-terminus of the transmembrane domain is less than 2, 3, 4, 5, 6, 7, 8, 9, or 10 amino acids long so that the intracellular domain derived from the first polypeptide is close to the C-terminus of the transmembrane domain. In certain embodiments, the peptide linker between the intracellular domain derived from the first polypeptide and the C-terminus of the transmembrane domain is in a length that the intracellular domain derived from the first polypeptide is close to the C-terminus of the transmembrane domain and/or close to the membrane of the engineered immune cell. In certain embodiments, the peptide linker between the intracellular domain derived from the first polypeptide and the C-terminus of the transmembrane domain is less than 2, 3, 4, 5, 6, 7, 8, 9, or 10 amino acids long so that the intracellular domain derived from the first polypeptide is capable of activating PI3K at the site of membrane. In certain embodiments, the peptide linker between the intracellular domain derived from the first polypeptide and the C-terminus of the transmembrane domain is in a length that the intracellular domain derived from the first polypeptide is capable of activating PI3K at the site of membrane. In certain embodiments, the peptide linker between the intracellular domain derived from the first polypeptide and the C-terminus of the transmembrane domain is GGGS (SEQ ID NO:153). In certain embodiments, the peptide linker between the intracellular domain derived from the first polypeptide and the C-terminus of the transmembrane domain is one or more DNA restriction endonuclease sites. Exemplary DNA restriction endonuclease sites include, but are not limited to, NotI, BglII, BamhI, EcoRI, NheI, XhoI, Pacd, Sacd, SacII, XbaI, SalI, and ClaI.

In certain embodiments, the intracellular signaling domain of the CAR comprises an amino acid sequence at least 75%, 80%, 85%, 90%, 95%, 96%, 97%, 98%, 99%, or more identical to the amino acid sequence of SEQ ID NO:20, SEQ ID NO:53, or SEQ ID NO:21. In certain embodiments, the intracellular signaling domain of the CAR comprises the amino acid sequence of SEQ ID NO:20, SEQ ID NO:53, or SEQ ID NO:21. In certain embodiments, the intracellular signaling domain of the CAR comprises an amino acid sequence at least 75%, 80%, 85%, 90%, 95%, 96%, 97%, 98%, 99%, or more identical to the amino acid sequence of SEQ ID NO:20. In certain embodiments, the intracellular signaling domain of the CAR comprises the amino acid sequence of SEQ ID NO:20.

In certain embodiments, the intracellular signaling domain of the CAR further comprises an intracellular domain derived from a third polypeptide, wherein the third polypeptide is different from the first polypeptide. In certain embodiments, the second polypeptide and the third polypeptide are the same polypeptide. In certain embodiments, the second polypeptide and the third polypeptide are different polypeptides. In certain embodiments, the third polypeptide is a signaling lymphocytic activation molecule family (SLAMF) receptor, CD2, CD28H, DNAM1, CD28, 4-1BB, OX40, KIR-S, NKG2C, or NKG2E. In certain embodiments, the SLAMF receptor is SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), or SLAMF9 (CD84H1, SF2001). In certain embodiments, the KIR-S is KIR2DS1, KIR2DS2, KIR2DS3, KIR2DS4, KIR2DS5 or KIR3DS1.

In some embodiments, the intracellular signaling domain of the CAR further comprises an intracellular domain derived from SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), SLAMF9 (CD84H1, SF2001), CD8, CD28, 4-1BB, OX40, 2B4, CD2, CD28H, DNAM1, KIR2DS1, KIR2DS2, KIR2DS3, KIR2DS4, KIR2DS5, KIR3DS1, NKG2C, or NKG2E. In some embodiments, the intracellular signaling domain of the CAR further comprises the intracellular domain (ICD) of SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), SLAMF9 (CD84H1, SF2001), CD8, CD28, 4-1BB, OX40, 2B4, CD2, CD28H, DNAM1, KIR2DS1, KIR2DS2, KIR2DS3, KIR2DS4, KIR2DS5, KIR3DS1, NKG2C, or NKG2E. In certain embodiments, the intracellular signaling domain of the CAR comprises an amino acid sequence at least 75%, 80%, 85%, 90%, 95%, 96%, 97%, 98%, 99%, or more identical to the amino acid sequence of SEQ ID NO:13, SEQ ID NO:25, SEQ ID NO:29, SEQ ID NO:33, SEQ ID NO:37, SEQ ID NO:41, SEQ ID NO:57, SEQ ID NO:61, SEQ ID NO:65, SEQ ID NO:69, SEQ ID NO:73, SEQ ID NO:77, SEQ ID NO:79, or SEQ ID NO:83. In certain embodiments, the intracellular signaling domain of the CAR comprises the amino acid sequence of SEQ ID NO:13, SEQ ID NO:25, SEQ ID NO:29, SEQ ID NO:33, SEQ ID NO:37, SEQ ID NO:41, SEQ ID NO:57, SEQ ID NO:61, SEQ ID NO:65, SEQ ID NO:69, SEQ ID NO:73, SEQ ID NO:77, SEQ ID NO:79, or SEQ ID NO:83.

In certain embodiments, the intracellular signaling domain of the CAR further comprises an intracellular domain derived from CD3zeta. In certain embodiments, the intracellular signaling domain of the CAR further comprises the intracellular domain of CD3zeta (CD3zeta ICD). In certain embodiments, the intracellular signaling domain of the CAR comprises an amino acid sequence at least 75%, 80%, 85%, 90%, 95%, 96%, 97%, 98%, 99%, or more identical to the amino acid sequence of SEQ ID NO:19. In certain embodiments, the intracellular signaling domain of the CAR comprises the amino acid sequence of SEQ ID NO:19.

In certain embodiments, the intracellular signaling domain of the CAR further comprises an intracellular domain derived from SLAM-associated protein (SAP). In certain embodiments, the intracellular signaling domain of the CAR comprises an amino acid sequence at least 75%, 80%, 85%, 90%, 95%, 96%, 97%, 98%, 99%, or more identical to the amino acid sequence of SEQ ID NO:86. In certain embodiments, the intracellular signaling domain of the CAR comprises the amino acid sequence of SEQ ID NO:86.

In certain embodiments, the intracellular signaling domain of the CAR further comprises an intracellular domain derived from EAT2. In certain embodiments, the intracellular signaling domain of the CAR comprises an amino acid sequence at least 75%, 80%, 85%, 90%, 95%, 96%, 97%, 98%, 99%, or more identical to the amino acid sequence of SEQ ID NO:87. In certain embodiments, the intracellular signaling domain of the CAR comprises the amino acid sequence of SEQ ID NO:87.

In certain embodiments, the intracellular signaling domain of the CAR further comprises a peptide linker derived from P2A, T2A, or E2A. In certain embodiments, the intracellular signaling domain of the CAR further comprises P2A, T2A, or E2A. In certain embodiments, the intracellular signaling domain of the CAR comprises an amino acid sequence at least 75%, 80%, 85%, 90%, 95%, 96%, 97%, 98%, 99%, or more identical to the amino acid sequence of SEQ ID NO:1, SEQ ID NO:2 or SEQ ID NO:3. In certain embodiments, the intracellular signaling domain of the CAR comprises the amino acid sequence of SEQ ID NO:1, SEQ ID NO:2 or SEQ ID NO:3. In certain embodiments, the intracellular signaling domain of the CAR further comprises a peptide linker derived from P2A. In certain embodiments, the intracellular signaling domain of the CAR further comprises P2A. In certain embodiments, the intracellular signaling domain of the CAR comprises an amino acid sequence at least 75%, 80%, 85%, 90%, 95%, 96%, 97%, 98%, 99%, or more identical to the amino acid sequence of SEQ ID NO:1. In certain embodiments, the intracellular signaling domain of the CAR comprises the amino acid sequence of SEQ ID NO:1.

In certain embodiments, the intracellular domain derived from CD3zeta is linked to the intracellular domain derived from SAP or the intracellular domain derived from EAT2 directly or via a peptide linker. In certain embodiments, the intracellular domain derived from CD3zeta is linked to the intracellular domain derived from SAP or the intracellular domain derived from EAT2 via P2A, T2A, or E2A.

In certain embodiments, without being bound by any particular theory, an intracellular domain derived from SAP or an intracellular domain derived from EAT2 can facilitate the CAR to trigger a complete activation signal and completely overcome the inhibitory receptor signaling.

In certain embodiments, the intracellular signaling domain of the CAR comprises a primary signaling domain and one or more co-stimulatory signaling domains. In certain embodiments, the primary signaling domain and the one or more co-stimulatory signaling domains are linked directly to each other or linked to each other via peptide linker(s).

In certain embodiments, the intracellular signaling domain of the CAR comprises a primary signaling domain comprising an intracellular domain derived from CD3zeta. In certain embodiments, the intracellular signaling domain of the CAR comprises a primary signaling domain comprising the intracellular domain of CD3zeta (CD3zeta ICD). In certain embodiments, the intracellular signaling domain of the CAR comprises a primary signaling domain comprising an amino acid sequence at least 75%, 80%, 85%, 90%, 95%, 96%, 97%, 98%, 99%, or more identical to the amino acid sequence of SEQ ID NO:19. In certain embodiments, the intracellular signaling domain of the CAR comprises a primary signaling domain comprising the amino acid sequence of SEQ ID NO:19.

In certain embodiments, the intracellular signaling domain of the CAR comprises a co-stimulatory signaling domain comprising an intracellular domain derived from a first polypeptide that is DAP10, DNAM1, or DAP12. In certain embodiments, the intracellular signaling domain of the CAR comprises a co-stimulatory signaling domain comprising the intracellular domain of DAP10, DNAM1, or DAP12. In certain embodiments, the intracellular signaling domain of the CAR comprises a co-stimulatory signaling domain comprising an amino acid sequence at least 75%, 80%, 85%, 90%, 95%, 96%, 97%, 98%, 99%, or more identical to the amino acid sequence of SEQ ID NO:20, SEQ ID NO:53, or SEQ ID NO:21. In certain embodiments, the intracellular signaling domain of the CAR comprises a co-stimulatory signaling domain comprising the amino acid sequence of SEQ ID NO:20, SEQ ID NO:53, or SEQ ID NO:21. In certain embodiments, the intracellular signaling domain of the CAR comprises a co-stimulatory signaling domain comprising an intracellular domain derived from DAP10. In certain embodiments, the intracellular signaling domain of the CAR comprises a co-stimulatory signaling domain comprising the intracellular domain of DAP10 (DAP10 ICD). In certain embodiments, the intracellular signaling domain of the CAR comprises a co-stimulatory signaling domain comprising an amino acid sequence at least 75%, 80%, 85%, 90%, 95%, 96%, 97%, 98%, 99%, or more identical to the amino acid sequence of SEQ ID NO:20. In certain embodiments, the intracellular signaling domain of the CAR comprises a co-stimulatory signaling domain comprising the amino acid sequence of SEQ ID NO:20.

In certain embodiments, the intracellular signaling domain of the CAR comprises a co-stimulatory signaling domain comprising an intracellular domain derived from a third polypeptide that is different from the first polypeptide. In certain embodiments, the intracellular signaling domain of the CAR comprises a co-stimulatory signaling domain comprising an intracellular domain derived from SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), SLAMF9 (CD84H1, SF2001), CD8, CD28, 4-1BB, OX40, 2B4, CD2, CD28H, DNAM1, KIR2DS1, KIR2DS2, KIR2DS3, KIR2DS4, KIR2DS5, KIR3DS1, NKG2C, or NKG2E. In certain embodiments, the intracellular signaling domain of the CAR comprises a co-stimulatory signaling domain comprising the intracellular domain (ICD) of SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), SLAMF9 (CD84H1, SF2001), CD8, CD28, 4-1BB, OX40, 2B4, CD2, CD28H, DNAM1, KIR2DS1, KIR2DS2, KIR2DS3, KIR2DS4, KIR2DS5, KIR3DS1, NKG2C, or NKG2E. In certain embodiments, the intracellular signaling domain of the CAR comprises a co-stimulatory signaling domain comprising an amino acid sequence at least 75%, 80%, 85%, 90%, 95%, 96%, 97%, 98%, 99%, or more identical to the amino acid sequence of SEQ ID NO:13, SEQ ID NO:25, SEQ ID NO:29, SEQ ID NO:33, SEQ ID NO:37, SEQ ID NO:41, SEQ ID NO:57, SEQ ID NO:61, SEQ ID NO:65, SEQ ID NO:69, SEQ ID NO:73, SEQ ID NO:77, SEQ ID NO:79, or SEQ ID NO:83. In certain embodiments, the intracellular signaling domain of the CAR comprises a co-stimulatory signaling domain comprising the amino acid sequence of SEQ ID NO:13, SEQ ID NO:25, SEQ ID NO:29, SEQ ID NO:33, SEQ ID NO:37, SEQ ID NO:41, SEQ ID NO:57, SEQ ID NO:61, SEQ ID NO:65, SEQ ID NO:69, SEQ ID NO:73, SEQ ID NO:77, SEQ ID NO:79, or SEQ ID NO:83.

In certain embodiments, the intracellular signaling domain of the CAR comprises a co-stimulatory signaling domain comprising an intracellular domain derived from SAP or EAT2. In certain embodiments, the intracellular signaling domain of the CAR comprises a co-stimulatory signaling domain comprising SAP or EAT. In certain embodiments, the intracellular signaling domain of the CAR comprises a co-stimulatory signaling domain comprising an amino acid sequence at least 75%, 80%, 85%, 90%, 95%, 96%, 97%, 98%, 99%, or more identical to the amino acid sequence of SEQ ID NO:86 or SEQ ID NO:87. In certain embodiments, the intracellular signaling domain of the CAR comprises a co-stimulatory signaling domain comprising the amino acid sequence of SEQ ID NO:86 or SEQ ID NO:87.

### 5.2.5 Hinge Domain

The CARs provided herein may comprise a hinge domain that is located between the extracellular antigen binding domain and the transmembrane domain. In certain embodiments, the CAR further comprises a hinge domain between the C-terminus of the extracellular antigen binding domain and the N-terminus of the transmembrane domain. In certain embodiments, the hinge domain of the CAR comprises a hinge domain derived from the second polypeptide provided herein.

In some embodiments, the hinge domain is derived from SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), SLAMF9 (CD84H1, SF2001), CD8, CD28, 4-1BB, OX40, 2B4, CD2, CD28H, DNAM1, KIR2DS1, KIR2DS2, KIR2DS3, KIR2DS4, KIR2DS5, KIR3DS1, NKG2C, or NKG2E. In some embodiments, the hinge domain is the hinge domain of SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), SLAMF9 (CD84H1, SF2001), CD8, CD28, 4-1BB, OX40, 2B4, CD2, CD28H, DNAM1, KIR2DS1, KIR2DS2, KIR2DS3, KIR2DS4, KIR2DS5, KIR3DS1, NKG2C, or NKG2E. In certain embodiments, the hinge domain comprises an amino acid sequence at least 75%, 80%, 85%, 90%, 95%, 96%, 97%, 98%, 99%, or more identical to the amino acid sequence of SEQ ID NO:5, SEQ ID NO:7, SEQ ID NO:11, SEQ ID NO:15, SEQ ID NO:23, SEQ ID NO:27, SEQ ID NO:31, SEQ ID NO:35, SEQ ID NO:39, SEQ ID NO:43, SEQ ID NO:47, SEQ ID NO:51, SEQ ID NO:55, SEQ ID NO:59, SEQ ID NO:63, SEQ ID NO:67, SEQ ID NO:71, SEQ ID NO:75, SEQ ID NO:81, or SEQ ID NO:85. In certain embodiments, the hinge domain comprises the amino acid sequence of SEQ ID NO:5, SEQ ID NO:7, SEQ ID NO:11, SEQ ID NO:15, SEQ ID NO:23, SEQ ID NO:27, SEQ ID NO:31, SEQ ID NO:35, SEQ ID NO:39, SEQ ID NO:43, SEQ ID NO:47, SEQ ID NO:51, SEQ ID NO:55, SEQ ID NO:59, SEQ ID NO:63, SEQ ID NO:67, SEQ ID NO:71, SEQ ID NO:75, SEQ ID NO:81, or SEQ ID NO:85.

A hinge domain is an amino acid segment that is generally found between two domains of a protein and may allow for flexibility of the protein and movement of one or both of the domains relative to one another. Any amino acid sequence that provides such flexibility and movement of the extracellular antigen binding domain relative to the transmembrane domain of the effector molecule can be used.

The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. Hinge domains of any protein known in the art to comprise a hinge domain are compatible for use in the CARs provided herein. In some embodiments, the hinge domain is at least a portion of a hinge domain of a naturally occurring protein and confers flexibility to the chimeric receptor. In some embodiments, the hinge domain is a portion of the hinge domain of CD8a, e.g., a fragment containing at least 15 (e.g., 20, 25, 30, 35, or 40) consecutive amino acids of the hinge domain of CD8a.

Hinge domains of antibodies, such as an IgG, IgA, IgM, IgE, or IgD antibodies, are also compatible for use in the CAR described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptors described herein. In some embodiments, the hinge domain between the C-terminus of the extracellular ligand-binding domain of an Fc receptor and the N-terminus of the transmembrane domain is a peptide linker, such as a (GxS)n linker, wherein x and n, independently can be an integer between 3 and 12, including 3, 4, 5, 6, 7, 8, 9, 10, 11, 12, or more.

### 5.2.6 Peptide Linkers

In certain embodiments, the CAR provided herein comprises one or more peptide linkers. In certain embodiments, the peptide linker is less than 2, 3, 4, 5, 6, 7, 8, 9, or 10 amino acids long. In certain embodiments, the peptide linker is GGGS (SEQ ID NO:153). In certain embodiments, the peptide linker comprises one or more DNA restriction endonuclease sites. Exemplary DNA restriction endonuclease sites include, but are not limited to, NotI, BglII, BamhI, EcoRI, NheI, XhoI, Pacd, Sacd, SacII, XbaI, SalI, and ClaI.

In certain embodiments, the CAR provided herein comprises a peptide linker between the transmembrane domain and the intracellular signaling domain. In certain embodiments, the intracellular domain derived from the first polypeptide (i.e., DAP10, DNAM1, CD28H, CD2, DAP12, CD28, or OX40) is linked to the C-terminus of the transmembrane domain via a peptide linker. In certain embodiments, the peptide linker between the intracellular domain derived from the first polypeptide and the C-terminus of the transmembrane domain is less than 2, 3, 4, 5, 6, 7, 8, 9, or 10 amino acids long so that the peptide linker between the intracellular domain derived from the first polypeptide is close to the C-terminus of the transmembrane domain. In certain embodiments, the peptide linker between the intracellular domain derived from the first polypeptide and the C-terminus of the transmembrane domain is GGGS (SEQ ID NO:153). In certain embodiments, the peptide linker between the intracellular domain derived from the first polypeptide and the C-terminus of the transmembrane domain is one or more DNA restriction endonuclease sites. Exemplary DNA restriction endonuclease sites include, but are not limited to, NotI, BglII, BamhI, EcoRI, NheI, XhoI, PacI, SacI, SacII, XbaI, SalI, and ClaI.

In certain embodiments, the CAR provided herein comprises one or more peptide linkers within the intracellular signaling domain. In certain embodiments, the one or more peptide linkers within the intracellular signaling domain is between intracellular domains derived from polypeptides. In certain embodiments, an intracellular domain derived from CD3zeta is linked to an intracellular domain derived from SAP or an intracellular domain derived from EAT2 via a peptide linker.

Each peptide linker in a CAR may have the same or different length and/or sequence depending on the structural and/or functional features of the single domain antibodies and/or the various domains. Each peptide linker may be selected and optimized independently. The length, the degree of flexibility and/or other properties of the peptide linker(s) used in the CARs may have some influence on properties, including but not limited to the affinity, specificity or avidity for one or more particular antigens or epitopes. For example, longer peptide linkers may be selected to ensure that two adjacent domains do not sterically interfere with one another. In some embodiments, a short peptide linker may be disposed between the transmembrane domain and the intracellular signaling domain of a CAR. In some embodiment, a peptide linker comprises flexible residues (such as glycine and serine) so that the adjacent domains are free to move relative to each other. For example, a glycine-serine doublet can be a suitable peptide linker.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. Exemplary flexible linkers include but not limited to glycine polymers (G)ₙ, glycine-serine polymers (including, for example, (GS)ₙ, (GSGGS)ₙ, (GGGS)ₙ, and (GGGGS)ₙ, where n is an integer of at least one), glycine-alanine polymers, alanine-serine polymers, and other flexible linkers known in the art. Exemplary peptide linkers are listed in the table below.

TABLE 3
Exemplary Peptide Linkers

| SEQ ID NO: | Sequence |
|---|---|
| 146 | (GS)ₙ, n is an integer including, e.g., 1, 2, 3, 4, 5, and 6. |
| 147 | (GSGGS)ₙ, n is an integer including, e.g., 1, 2, 3, 4, 5, and 6. |
| 148 | (GGGS)ₙ, n is an integer including, e.g., 1, 2, 3, 4, 5, and 6. |
| 149 | (GGGGS)ₙ, n is an integer including, e.g., 1, 2, 3, 4, 5, and 6. |
| 150 | GS |
| 151 | GSGS |
| 152 | GSGSGS |
| 153 | GGGS |
| 154 | GGGSGGGS |
| 155 | GSGGS |
| 156 | GSGGSGSGGS |
| 157 | GSGGGS |
| 158 | GGGGS |
| 159 | GGGGSGGGGS |
| 160 | GGRR |
| 161 | GGGGSGGGGSGGGGGGSGSGGGGS |
| 162 | GGRRGGGS |

Other linkers known in the art, for example, as described in WO2016014789, WO2015158671, WO2016102965, US20150299317, WO2018067992, U.S. Pat. No. 7,741,465, Colcher et al., J. Nat. Cancer Inst. 82:1191-1197 (1990), and Bird et al., Science 242:423-426 (1988) may also be included in the CARs provided herein, the disclosure of each of which is incorporated herein by reference.

### 5.3 Engineered Immune Cells

In another aspect, provided herein are engineered immune cells. In certain embodiments, provided herein is an engineered immune cell comprising the CAR provided herein.

In certain embodiments, provided herein is an engineered immune cell comprising a CAR, wherein the CAR comprises:
(i) an extracellular antigen binding domain;
(ii) a transmembrane domain; and
(iii) an intracellular signaling domain, and
wherein the engineered immune cell further comprises an exogenously introduced polynucleotide encoding an intracellular domain derived from SLAM-associated protein (SAP), and wherein the engineered immune cell is an NK cell, an NKT cell, or a T cell.

In certain embodiments, provided herein is an engineered immune cell comprising a CAR, wherein the CAR comprises:
(i) an extracellular antigen binding domain;
(ii) a transmembrane domain; and
(iii) an intracellular signaling domain, and
wherein the engineered immune cell further comprises an exogenously introduced polynucleotide encoding an intracellular domain derived from Ewing's sarcoma-activated transcript-2 (EAT2), and wherein the engineered immune cell is an NK cell, an NKT cell, or a T cell.

In certain embodiments, the polynucleotide encodes the CAR in the engineered immune cell, and wherein the intracellular signaling domain of the CAR in the engineered immune cell comprises the intracellular domain derived from SAP or the intracellular domain derived from EAT2.

In certain embodiments, the intracellular signaling domain of the CAR in the engineered immune cell comprises an intracellular domain derived from a first polypeptide, and wherein the first polypeptide is DAP10, DNAM1, CD28H, CD2, DAP12, CD28, or OX40.

In certain embodiments, the intracellular domain of the first polypeptide is linked to the C-terminus of the transmembrane domain directly or via a peptide linker.

In certain embodiments, the transmembrane domain of the CAR in the engineered immune cell comprises a transmembrane domain derived from a second polypeptide. In certain embodiments, the intracellular signaling domain of the CAR in the engineered immune cell comprises a intracellular domain derived from a third polypeptide. In certain embodiments, the second polypeptide or the third polypeptide is a signaling lymphocytic activation molecule family (SLAMF) receptor, CD2, CD28H, DNAM1, CD28, 4-1BB, OX40, KIR-S, NKG2C, or NKG2E. In certain embodiments, the SLAMF receptor is SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), or SLAMF9 (CD84H1, SF2001). In certain embodiments, the KIR-S is KIR2DS1, KIR2DS2, KIR2DS3, KIR2DS4, KIR2DS5 or KIR3DS1.

In certain embodiments, intracellular signaling domain of the CAR in the engineered immune cell comprises an intracellular domain derived from CD3zeta. In certain embodiments, intracellular signaling domain of the CAR in the engineered immune cell further comprises an intracellular domain derived from SLAM-associated protein (SAP) or an intracellular domain derived from EAT2, and wherein the intracellular domain derived from CD3zeta is linked to the intracellular domain derived from SAP or the intracellular domain derived from EAT2 directly or via a peptide linker. In certain embodiments, intracellular domain derived from CD3zeta is linked to the intracellular domain derived from SAP or the intracellular domain derived from EAT2 via P2A, T2A, or E2A.

In certain embodiments, extracellular antigen binding domain of the CAR in the engineered immune cell comprises an scFv. In certain embodiments, extracellular antigen binding domain of the CAR binds to a tumor antigen or a fragment thereof.

In certain embodiments, the CAR in the engineered immune cell comprises an amino acid sequence at least 90%, at least 95%, or 100% identical to the amino acid sequence of SEQ ID NO:20.

In certain embodiments, the CAR in the engineered immune cell comprises an amino acid sequence at least 90%, at least 95%, or 100% identical to the amino acid sequence of SEQ ID NO:86.

In certain embodiments, the CAR in the engineered immune cell comprises an amino acid sequence at least 90%, at least 95%, or 100% identical to the amino acid sequence of SEQ ID NO:87.

In certain embodiments, the CAR in the engineered immune cell comprises an amino acid sequence at least 90%, at least 95%, or 100% identical to the amino acid sequence of SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, or SEQ ID NO:145.

In certain embodiments, the engineered immune cell is NK cell.

### 5.3.1 Exemplary Engineered Immune Cells

In certain embodiments, the engineered immune cell provided herein comprises a CAR comprising
(i) an extracellular antigen binding domain;
(ii) a transmembrane domain; and
(iii) an intracellular signaling domain, and
wherein the engineered immune cell further comprises an exogenously introduced polynucleotide encoding an intracellular domain derived from SLAM-associated protein (SAP) or Ewing's sarcoma-activated transcript-2 (EAT2), and wherein the engineered immune cell is an NK cell, an NKT cell, or a T cell.

In certain embodiments, the engineered immune cell provided herein comprises any CAR known in the art and an exogenously introduced polynucleotide encoding an intracellular domain derived from SLAM-associated protein (SAP). In certain embodiments, the engineered immune cell provided herein comprises any CAR known in the art and an exogenously introduced polynucleotide encoding SLAM-associated protein (SAP). In certain embodiments, the engineered immune cell provided herein comprises any CAR known in the art and an exogenously introduced polynucleotide encoding an amino acid sequence at least 75%, 80%, 85%, 90%, 95%, 96%, 97%, 98%, 99%, or more identical to the amino acid sequence of SEQ ID NO:86. In certain embodiments, the engineered immune cell provided herein comprises any CAR known in the art and an exogenously introduced polynucleotide encoding the amino acid sequence of SEQ ID NO:86.

In certain embodiments, the engineered immune cell provided herein comprises any CAR known in the art and an exogenously introduced polynucleotide encoding an intracellular domain derived from Ewing's sarcoma-activated transcript-2 (EAT2). In certain embodiments, the engineered immune cell provided herein comprises any CAR known in the art and an exogenously introduced polynucleotide encoding Ewing's sarcoma-activated transcript-2 (EAT2). In certain embodiments, the engineered immune cell provided herein comprises any CAR known in the art and an exogenously introduced polynucleotide encoding an amino acid sequence at least 75%, 80%, 85%, 90%, 95%, 96%, 97%, 98%, 99%, or more identical to the amino acid sequence of SEQ ID NO:87. In certain embodiments, the engineered immune cell provided herein comprises any CAR known in the art and an exogenously introduced polynucleotide encoding the amino acid sequence of SEQ ID NO:87.

In certain embodiments, any CAR known in the art and an intracellular domain derived from SLAM-associated protein (SAP) are both expressed in the engineered immune cell provided herein. In certain embodiments, any CAR known in the art and SAP are both expressed in the engineered immune cell provided herein. In certain embodiments, any CAR known in the art and an amino acid sequence at least 75%, 80%, 85%, 90%, 95%, 96%, 97%, 98%, 99%, or more identical to the amino acid sequence of SEQ ID NO:86 are both expressed in the engineered immune cell provided herein. In certain embodiments, any CAR known in the art and the amino acid sequence of SEQ ID NO:86 are both expressed in the engineered immune cell provided herein.

In certain embodiments, any CAR known in the art and an intracellular domain derived from Ewing's sarcoma-activated transcript-2 (EAT2) are both expressed in the engineered immune cell provided herein. In certain embodiments, any CAR known in the art and EAT2 are both expressed in the engineered immune cell provided herein. In certain embodiments, any CAR known in the art and an amino acid sequence at least 75%, 80%, 85%, 90%, 95%, 96%, 97%, 98%, 99%, or more identical to the amino acid sequence of SEQ ID NO:87 are both expressed in the engineered immune cell provided herein. In certain embodiments, any CAR known in the art and the amino acid sequence of SEQ ID NO:87 are both expressed in the engineered immune cell provided herein.

In certain embodiments, the polynucleotide encoding an intracellular domain derived from SLAM-associated protein (SAP) or an intracellular domain derived from Ewing's sarcoma-activated transcript-2 (EAT2) also encodes the CAR. In certain embodiments, the CAR and the intracellular domain derived from SAP are encoded by the same polynucleotide. In certain embodiments, the CAR and the intracellular domain derived from EAT2 are encoded by the same polynucleotide.

In certain embodiments, an intracellular domain derived from SLAM-associated protein (SAP) or an intracellular domain derived from EAT2 is part of the CAR in the engineered immune cell. In certain embodiments, the engineered immune cell provided herein comprises a CAR, from N-terminus to C-terminus, comprising
(i) an extracellular antigen binding domain;
(ii) optionally a hinge domain that can be, but is not limited to, a hinge domain derived from a second polypeptide;
(iii) a transmembrane domain, optionally comprising an transmembrane domain derived from a second polypeptide;
(iv) optionally a peptide linker that can be, but is not limited to, a peptide linker provided herein;
(v) an intracellular signaling domain comprising an intracellular domain derived from SAP or an intracellular domain derived from EAT2.

In certain embodiments, the intracellular signaling domain of the CAR in the engineered immune cell further comprises one or more elements that are
(v)-(a) an intracellular domain derived from a first polypeptide that is DAP10, DNAM1, CD28H, CD2, DAP12, CD28, or OX40;
(v)-(b) an intracellular domain derived from a third polypeptide;
(v)-(c) an intracellular domain derived from CD3zeta; and
(v)-(d) a peptide linker, that can be, but is not limited to, P2A, T2A, or E2A.

In certain embodiments, the second polypeptide is different from the first polypeptide. In certain embodiments, the third polypeptide is different from the first polypeptide. In certain embodiments, the second polypeptide and the third polypeptide are the same polypeptide. In certain embodiments, the second polypeptide and the third polypeptide are different polypeptides. In certain embodiments, the second polypeptide or the third polypeptide is a signaling lymphocytic activation molecule family (SLAMF) receptor, CD2, CD28H, DNAM1, CD28, 4-1BB, OX40, KIR-S, NKG2C, or NKG2E. In certain embodiments, the SLAMF receptor is SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), or SLAMF9 (CD84H1, SF2001). In certain embodiments, the KIR-S is KIR2DS1, KIR2DS2, KIR2DS3, KIR2DS4, KIR2DS5 or KIR3DS1.

In certain embodiments, the engineered immune cell is an NK cell. In certain embodiments, the engineered immune cell is an NKT cell. In certain embodiments, the engineered immune cell is a T cell, such as CAR-T, TIL, TCR-T, gamma-deltaT, and double negative DN-T.

### 5.4 Polypeptides

In yet another aspect, provided herein are polypeptides. In certain embodiments, provided herein is a polypeptide comprising the CAR provided herein.

In certain embodiments, provided herein is a polypeptide comprising a chimeric antigen receptor (CAR) and an intracellular domain derived from SLAM-associated protein (SAP), wherein the CAR comprises:
(i) an extracellular antigen binding domain;
(ii) a transmembrane domain; and
(iii) an intracellular signaling domain, and
wherein the CAR is linked to an intracellular domain derived from SAP via a peptide linker.

In certain embodiments, provided herein is a polypeptide comprising a chimeric antigen receptor (CAR) an intracellular domain derived from EAT2, wherein the CAR comprises:
(i) an extracellular antigen binding domain;
(ii) a transmembrane domain; and
(iii) an intracellular signaling domain, and
wherein the CAR is linked to an intracellular domain derived from SAP via a peptide linker.

In certain embodiments, the CAR is linked to the intracellular domain derived from SAP or the intracellular domain derived from EAT2 via P2A, T2A, or E2A.

In certain embodiments, the polypeptide comprises an amino acid sequence at least 90%, at least 95%, or 100% identical to the amino acid sequence of SEQ ID NO:86 or SEQ ID NO:87.

In certain embodiments, the polypeptide comprises an amino acid sequence at least 90%, at least 95%, or 100% identical to the amino acid sequence of SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, or SEQ ID NO:145.

### 5.5 Polynucleotides and Vectors

In yet another aspect, provided herein are polynucleotide. In certain embodiments, provided herein is a polynucleotide encoding the CAR provided herein. In certain embodiments, provided herein is a polynucleotide encoding the polypeptide provided herein.

In certain embodiments, the polynucleotide encodes an amino acid sequence at least 90%, at least 95%, or 100% identical to the amino acid sequence of SEQ ID NO:86 or SEQ ID NO:87.

In certain embodiments, the polynucleotide encodes an amino acid sequence at least 90%, at least 95%, or 100% identical to the amino acid sequence of SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, or SEQ ID NO:145.

The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

The present application further relates to variants of the polynucleotides described herein, wherein the variant encodes, for example, fragments, analogs, and/or derivatives of the CAR provided herein or the polypeptide provided herein. In certain embodiments, the present application provides a polynucleotide comprising a polynucleotide having a nucleotide sequence at least about 75% identical, at least about 80% identical, at least about 85% identical, at least about 90% identical, at least about 95% identical, and in some embodiments, at least about 96%, 97%, 98% or 99% identical to a polynucleotide encoding the CAR provided herein or the polypeptide provided herein. As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In yet another aspect, provided herein are vectors. In certain embodiments, provided herein is a vector comprising the polynucleotide provided herein.

In an embodiment, the polynucleotide can be incorporated into a recombinant expression vector. The present application provides recombinant expression vectors comprising any of the polynucleotide provided herein. As used herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors described herein are not naturally-occurring as a whole; however, parts of the vectors can be naturally-occurring. The described recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. The non-naturally occurring or altered nucleotides or internucleotide linkages do not hinder the transcription or replication of the vector.

### 5.6 Pharmaceutical Composition

In yet another aspect, provided herein are pharmaceutical compositions. In certain embodiments, provided herein is a pharmaceutical composition comprising
(i) the CAR provided herein, the engineered immune cell provided herein, the polypeptide provided herein, the polynucleotide provided herein, or the vector provided herein; and
(ii) a pharmaceutically acceptable excipient.

In certain embodiments, provided herein is a pharmaceutical composition comprising
(i) the CAR provided herein, and
(ii) a pharmaceutically acceptable excipient.

In certain embodiments, provided herein is a pharmaceutical composition comprising
(i) the engineered immune cell provided herein; and
(ii) a pharmaceutically acceptable excipient.

In certain embodiments, provided herein is a pharmaceutical composition comprising
(i) the polypeptide provided herein; and
(ii) a pharmaceutically acceptable excipient.

In certain embodiments, provided herein is a pharmaceutical composition comprising
(i) the polynucleotide provided herein; and
(ii) a pharmaceutically acceptable excipient.

In certain embodiments, provided herein is a pharmaceutical composition comprising
(i) the vector provided herein; and
(ii) a pharmaceutically acceptable excipient.

In certain embodiments, provided herein is a pharmaceutical composition comprising the CAR provided herein, the engineered immune cell provided herein, the polypeptide provided herein, the polynucleotide provided herein, or the vector provided herein. In certain embodiments, provided herein is a pharmaceutical composition comprising the CAR provided herein. In certain embodiments, provided herein is a pharmaceutical composition comprising the engineered immune cell provided herein. In certain embodiments, provided herein is a pharmaceutical composition comprising the polypeptide provided herein. In certain embodiments, provided herein is a pharmaceutical composition comprising the polynucleotide provided herein. In certain embodiments, provided herein is a pharmaceutical composition comprising the vector provided herein.

In certain embodiments, provided herein is a pharmaceutical composition comprising an effective amount of the CAR provided herein, an effective amount of the engineered immune cell provided herein, an effective amount of the polypeptide provided herein, an effective amount of the polynucleotide provided herein, or an effective amount of the vector provided herein. In certain embodiments, provided herein is a pharmaceutical composition comprising an effective amount of the CAR provided herein. In certain embodiments, provided herein is a pharmaceutical composition comprising an effective amount of the engineered immune cell provided herein. In certain embodiments, provided herein is a pharmaceutical composition comprising an effective amount of the polypeptide provided herein. In certain embodiments, provided herein is a pharmaceutical composition comprising an effective amount of the polynucleotide provided herein. In certain embodiments, provided herein is a pharmaceutical composition comprising an effective amount of the vector provided herein.

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In a specific embodiment, the term “excipient” can also refer to a diluent, adjuvant (e.g., Freunds' adjuvant (complete or incomplete), carrier or vehicle. Pharmaceutical excipients can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid excipients. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical excipients are described in Remington's Pharmaceutical Sciences (1990) Mack Publishing Co., Easton, PA. Such compositions contain a prophylactically or therapeutically effective amount of the active ingredient provided herein, such as in purified form, together with a suitable amount of excipient so as to provide the form for proper administration to the patient. The formulation should suit the mode of administration.

In some embodiments, the choice of excipient is determined in part by the particular cell, binding molecule, and/or antibody, and/or by the method of administration. Accordingly, there are a variety of suitable formulations.

Typically, acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers may be used to control the pH in a range which optimizes the therapeutic effectiveness, especially if stability is pH dependent. Suitable buffering agents for use with the present disclosure include both organic and inorganic acids and salts thereof. For example, citrate, phosphate, succinate, tartrate, fumarate, gluconate, oxalate, lactate, acetate. Additionally, buffers may comprise histidine and trimethylamine salts such as Tris.

Preservatives may be added to retard microbial growth. Suitable preservatives for use with the present disclosure include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intra-molecular interactions. Exemplary tonicity agents include polyhydric sugar alcohols, trihydric or higher sugar alcohols, such as glycerin, erythritol, arabitol, xylitol, sorbitol and mannitol.

Additional exemplary excipients include: (1) bulking agents, (2) solubility enhancers, (3) stabilizers and (4) agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) may be present to help solubilize the therapeutic agent as well as to protect the therapeutic protein against agitation-induced aggregation, which also permits the formulation to be exposed to shear surface stress without causing denaturation of the active therapeutic protein or antibody. Suitable non-ionic surfactants include, e.g., polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In order for the pharmaceutical compositions to be used for in vivo administration, they are preferably sterile. The pharmaceutical composition may be rendered sterile by filtration through sterile filtration membranes. The pharmaceutical compositions herein generally can be placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

In another embodiment, a pharmaceutical composition can be provided as a controlled release or sustained release system. In one embodiment, a pump may be used to achieve controlled or sustained release (see, e.g., Sefton, Crit. Ref. Biomed. Eng. 14:201-40 (1987); Buchwald et al., Surgery 88:507-16 (1980); and Saudek et al., N. Engl. J. Med. 321:569-74 (1989)). In another embodiment, polymeric materials can be used to achieve controlled or sustained release of a prophylactic or therapeutic agent (e.g., a fusion protein as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 23:61-126 (1983); Levy et al., Science 228:190-92 (1985); During et al., Ann. Neurol. 25:351-56 (1989); Howard et al., J. Neurosurg. 71:105-12 (1989); U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, Science 249:1527-33 (1990). Any technique known to one of skill in the art can be used to produce sustained release formulations comprising one or more agents as described herein (see, e.g., U.S. Pat. No. 4,526,938, PCT publication Nos. WO 91/05548 and WO 96/20698, Ning et al., Radiotherapy & Oncology 39:179-89 (1996); Song et al., PDA J. of Pharma. Sci. & Tech. 50:372-97 (1995); Cleek et al., Pro. Int'l. Symp. Control. Rel. Bioact. Mater. 24:853-54 (1997); and Lam et al., Proc. Int'l. Symp. Control Rel. Bioact. Mater. 24:759-60 (1997)).

The active ingredients may also be entrapped in microcapsules prepared, for example, by coascervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition.

Various compositions and delivery systems are known and can be used with the therapeutic agents provided herein, including, but not limited to, encapsulation in liposomes, microparticles, microcapsules, recombinant cells capable of expressing the single domain antibody or therapeutic molecule provided herein, construction of a nucleic acid as part of a retroviral or other vector, etc.

In some embodiments, the pharmaceutical composition provided herein contains the binding molecules and/or cells in amounts effective to treat or prevent the disease or disorder, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined.

### 5.7 Therapeutic Methods and Uses

In yet another aspect, provided herein are methods of treating a disease or disorder in a subject. In certain embodiments, provided herein is a method of treating a disease or disorder in a subject, comprising administering to the subject an effective amount of the engineered immune cell provided herein or the pharmaceutical composition provided herein.

In certain embodiments, the method of treating a disease or disorder in a subject, comprises administering to the subject an effective amount of the engineered immune cell provided herein. In certain embodiments, the method of treating a disease or disorder in a subject, comprises administering to the subject an effective amount of the pharmaceutical composition provided herein.

As provided herein, the engineered immune cells provided herein are suitable for use as a cell-based therapy or therapies, such as in a method of treating cancer. In certain embodiments, treatment with the engineered immune cell provided herein or the pharmaceutical composition provided herein results in one, two, or more, or all of the following: (1) a reduction in severity, progression, spread, and/or frequency of one or more symptoms, (2) elimination of one or more symptoms and/or underlying cause, (3) prevention of the occurrence of one or more symptoms and/or their underlying cause, and (4) improvement or remediation of damage. In specific embodiments, treatment includes therapeutic treatment as well as prophylactic, or suppressive measures for the condition, disease or disorder.

Such methods and uses include therapeutic methods and uses, for example, involving administration of the molecules, cells, or compositions containing the same, to a subject having a disease, condition, or disorder. In some embodiments, the molecule, cell, and/or composition is administered in an effective amount to effect treatment of the disease or disorder. Uses include uses of the engineered immune cell provided herein or the pharmaceutical composition provided herein in such methods and treatments, and in the preparation of a medicament in order to carry out such therapeutic methods. In some embodiments, the methods are carried out by administering the engineered immune cell provided herein or the pharmaceutical composition, to the subject having or suspected of having the disease or condition. In some embodiments, the methods thereby treat the disease or disorder in the subject.

In some embodiments, the treatment provided herein cause complete or partial amelioration or reduction of a disease or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms include, but do not imply, complete curing of a disease or complete elimination of any symptom or effect(s) on all symptoms or outcomes.

As used herein, in some embodiments, the treatment provided herein delay development of a disease or disorder, e.g., defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease or disorder. For example, a late stage cancer, such as development of metastasis, may be delayed.

In some embodiments, the methods include administration of the engineered immune cell provided herein or the pharmaceutical composition provided herein to a subject, tissue, or cell, such as one having, at risk for, or suspected of having the disease or disorder.

In some embodiments, the subject, to whom the engineered immune cell provided herein or the pharmaceutical composition provided herein are administered is a primate, such as a human. The subject can be male or female and can be any suitable age, including infant, juvenile, adolescent, adult, and geriatric subjects. In some examples, the subject is a validated animal model for disease, adoptive cell therapy, and/or for assessing toxic outcomes.

The engineered immune cell provided herein or the pharmaceutical composition provided herein, can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration.

The amount of a prophylactic or therapeutic agent provided herein that is effective in the prevention and/or treatment of a disease or condition can be determined by standard clinical techniques. Effective doses may be extrapolated from dose-response curves derived from in vitro or animal model test systems. For the prevention or treatment of disease, the appropriate dosage of the binding molecule or cell may depend on the type of disease or disorder to be treated, the type of binding molecule, the severity and course of the disease or disorder, whether the therapeutic agent is administered for preventive or therapeutic purposes, previous therapy, the patient's clinical history and response to the agent, and the discretion of the attending physician. The compositions, molecules and cells are in some embodiments suitably administered to the patient at one time or over a series of treatments.

In the context of engineered immune cells, in some embodiments, a subject may be administered the range of about one million to about 100 billion cells and/or that amount of cells per kilogram of body weight. In some embodiments, wherein the pharmaceutical composition comprises any one of the engineered immune cells described herein, the pharmaceutical composition is administered at a dosage of at least about any of 10⁴, 10⁵, 10⁶, 10⁷, 10⁸, or 10⁹ cells/kg of body weight of the individual. Dosages may vary depending on attributes particular to the disease or disorder and/or patient and/or other treatments.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once or multiple times during a dosing cycle. A dosing cycle can be, e.g., 1, 2, 3, 4, 5 or more week(s), or 1, 2, 3, 4, 5, or more month(s). The optimal dosage and treatment regime for a particular patient can be determined by one skilled in the art of medicine by monitoring the patient for signs of disease and adjusting the treatment accordingly.

In some embodiments, the engineered immune cell provided herein or the pharmaceutical composition provided herein are administered as part of a combination treatment, such as simultaneously with or sequentially with, in any order, another therapeutic intervention, such as another antibody or engineered cell or receptor or agent, such as a cytotoxic or therapeutic agent.

In some embodiments, the engineered immune cell provided herein or the pharmaceutical composition provided herein are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells or antibodies are administered prior to the one or more additional therapeutic agents. In some embodiments, the cells or antibodies are administered after to the one or more additional therapeutic agents.

In other embodiments, the method or the use provided herein prevents a disease or disorder. In some embodiments, the disease or disorder is a CD19 associated disease or disorder. In some embodiments, the disease or disorder is a GPC3 associated disease or disorder.

In certain embodiments, the disease or disorder is cancer. The therapeutic method or use can be characterized according to the cancer to be treated. For example, in certain embodiments, the cancer is a hematologic malignancy or leukemia. In certain embodiments, the cancer is acute myeloid leukemia (AML), acute lymphoblastic leukemia (ALL), myelodysplasia, myelodysplastic syndromes, acute T-lymphoblastic leukemia, or acute promyelocytic leukemia, chronic myelomonocytic leukemia, or myeloid blast crisis of chronic myeloid leukemia.

Other exemplary cancers to be treated by the engineered immune cell provided herein or the pharmaceutical composition provided herein include breast cancer, ovarian cancer, esophageal cancer, bladder or gastric cancer, salivary duct carcinoma, salivary duct carcinomas, adenocarcinoma of the lung or aggressive forms of uterine cancer, such as uterine serous endometrial carcinoma. In some other embodiments, the cancer is brain cancer, breast cancer, cervical cancer, colon cancer, colorectal cancer, endometrial cancer, esophageal cancer, leukemia, lung cancer, liver cancer, melanoma, ovarian cancer, pancreatic cancer, rectal cancer, renal cancer, stomach cancer, testicular cancer, or uterine cancer. In yet other embodiments, the cancer is a squamous cell carcinoma, adenocarcinoma, small cell carcinoma, melanoma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well-differentiated carcinoma, or Wilms tumor.

In some other embodiments, the cancer to be treated is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

In certain embodiments, the cancer is solid tumor. In certain embodiments, the cancer is liver cancer.

### 5.8 Methods of Engineering an Immune Cell

In yet another aspect, provided herein are methods of engineering an immune cell. In certain embodiments, provided herein is a method of engineering an immune cell, comprising introducing into the immune cell the polynucleotide provided herein, the vector provided herein, or the polypeptide provided herein.

In certain embodiments, provided herein is a method of engineering an immune cell, comprising introducing into the immune cell the polynucleotide provided herein. In certain embodiments, provided herein is a method of engineering an immune cell, comprising introducing into the immune cell the vector provided herein. In certain embodiments, provided herein is a method of engineering an immune cell, comprising introducing into the immune cell the polypeptide provided herein.

In certain embodiments, the immune cell is an NK cell, an NKT cell, or a T cell. In certain embodiments, the immune cell is an NK cell.

In some embodiments, provided herein is a method of engineering an immune cell, comprising introducing into the immune cell a polynucleotide provided herein. In some embodiments, the method further comprises, selecting a immune cell for expression of the CAR encoded by the polynucleotide or the vector provided herein. In some embodiments, the method further comprises, selecting a immune cell for expression of the SAP or EAT2 encoded by the polynucleotide or the vector provided herein.

### 5.9 Assays

The practice of the embodiments provided herein can employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999); Glover, ed., DNA Cloning, Volumes I and 11(1985); Freshney, ed., Animal Cell Culture: Immobilized Cells and Enzymes (IRL Press, 1986); Immunochemical Methods in Cell and Molecular Biology (Academic Press, London); Scopes, Protein Purification: Principles and Practice (Springer Verlag, N.Y., 2d ed. 1987); and/or Janeway, et al. Immunobiology (Garland Science, 7th ed. 2008).

In certain embodiments, once the engineered immune cells are administered to a mammal (e.g., a human), the biological activity of the engineered immune cell populations is measured by any of a number of known methods. Parameters to assess include specific binding of an engineered or natural immune cell to antigen, in vivo, e.g., by imaging, or ex vivo, e.g., by ELISA or flow cytometry. In certain embodiments, the ability of the engineered immune cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of engineered immune cells also can be measured by assaying expression and/or secretion of certain cytokines, such as CD107a, IFNγ, IL-2, and TNF. In some aspects the biological activity is measured by assessing clinical outcome, such as reduction in tumor burden or load.

### 5.9.1 Cells, Cell Culture, and Cloning

Any cell known in the art can be used herein, for example NK92 cells, NALM6 cells, 293T cells, HUH7 cells, Human primary NK cells, and K562-CD137L cells. Human primary NK cells can be isolated from healthy human donors' peripheral blood. Any known cloning technology can be used herein.

### 5.9.2 In Vitro Killing, Degranulation Assays

The killing activity of an engineered immune cell can be evaluated using various known methods in the art. For example, 293T, 293T-CD19, NALM6, and Huh7 target cells can be genetically modified and stably expressed Luc2-GFP as described previously (Zhang et al. (2021) Ectopic activation of the miR-200c-EpCAM axis enhances antitumor T cell responses in models of adoptive cell therapy. Sci Transl Med. 13(611):eabg4328). To assess killing activities, CAR-NK cells can be co-incubated with target cells with different ratios for different time as indicated in figure legends. D-Luciferin can be used to measure luminescence emission spectra. Target cells without NK cells can be used as 100% live cell control. The killing ability can be determined based on the normalized spectrum and background subtraction and calculated using the equation killing %=(1−(exp/100% live control))*100 (Zhang et al. (2021) Ectopic activation of the miR-200c-EpCAM axis enhances antitumor T cell responses in models of adoptive cell therapy. Sci Transl Med. 13(611):eabg 4328).

The degranulation activity of an engineered immune cell can be evaluated using various known methods in the art. For example, to assess degranulation, fluorescently conjugated anti-CD107a antibody can be added to the NK-target cocultures and incubated, cells can then be washed with FACS buffer, and stained with anti-CD56 antibodies. CD56 stained CD107a+NK cells can be quantified by flow cytometry analysis (Beckman Coulter CytoFLEX LX).

### 5.9.3 Flow Cytometry Analysis of Intracellular Cytokines

The IFN production can be evaluated using various known methods in the art. For example, to assess IFN production, live NK cells can be co-incubated with target cells in U-bottom 96-well plates after spinning. Brefeldin A can be added to the cells and incubated. Cell surface markers can be stained, washed, and fixed with IC Fixation Buffer (eBioscience), and washed and stained with cytokine antibodies using permeabilization buffer (eBioscience), and then analyzed by flow cytometry.

### 5.9.4 Duolink PLA

PLA assays can be performed using the in situ Duolink PLA red kit (Sigma-Aldrich) according to the manufacturer's instructions. The NK cells can be attached to the poly-l-lysine glass, target cells can then be added and incubated. Unattached cells can be removed by washing with PBS. slides can be fixed and stained with anti-GFP (Rabbit IgG) and anti-DAP10 (Mouse IgG), washed, and stained with anti-rabbit and anti-mouse PLA probes, followed by probe DNA ligation, amplification, Fluorescent staining can be then performed and observed using confocal microscopy,

### 5.9.5 Retroviral Transduction

Any known method for NK cell transduction can be used herein. For example, for NK cell transduction, Phoenix A cells can be transfected with expression vectors together with the packaging vector GagPol using the calcium method. Filtered viral supernatant, polybrene and IL-2 can be mixed with NK line cells or 3-day expanded primary NK cells in 6-well plates. The plates cam be centrifuged. The transduced NK line cells can be cultured with respective media for in vitro experiments, the transduced primary NK cells can be co-cultured with inactivated K562-CD137L and recombinant IL-2 and used for in vitro validation and adoptive cell therapy experiment in mice in the next step.

### 6. EXAMPLES

### 6.1 Example 1: Exemplary Structures of the Novel Complete Signaling NK-CAR (NK-csCAR) Using SLMAF Genes or Non-SLAMF Genes

ScFv-SLAMF hinge-SLAMF TM-DAP10 ICD-SLAMF ICD-CD3Z-P2A-SAP or EAT2
ScFv-non-SLAMF hinge-SLAMF TM-DAP10 ICD-non-SLAMF ICD-CD3Z-P2A-SAP or EAT2

As shown above, the exemplary elements of the novel complete signaling NK-CAR (NK-csCAR) include:
ScFv, to specifically recognize the target antigen;
SLAMF (such as 2B4, CD229, NTB-A, CRACC, CD84) or non-SLAMF, to provide the hinge and transmembrane (TM); the term “non-SLAMF,” as used herein, means polypeptides that are not SLAMF, such as CD2, CD28H, DNAM1.
DAP10 ICD, to provide the first co-stimulatory signal. This ICD is directly anchored in the intracellular inner membrane, the location of DAP10 ICD is surprisingly critical to enhance activation signaling effectively;
ICD of the same SLAMF or non-SLAMF gene, to provide the second co-stimulatory signal;
CD3zeta, to provide the third signal to phosphorylate SLP-76 on both Tyr113 and Tyr128 via pZAP70/pSyk-dependent pathway;
SAP or EAT2 as signal booster, the fourth signal, without being bound by any particular theory, to remove the effect of inhibitory receptor signals by competitively binding to the ITSM tail of SLAMF, and to prevent the binding of SHP to the pTry and recruit Fyn., which guarantees the activation signaling is complete, and the inhibitory receptor signaling barrier can be completely overcome.

We demonstrated that NK cells could be effectively activated using the natural hinge and transmembrane domain of the selected molecules. Unexpectedly, the first co-stimulatory signal provided by ICD of DAP10 was necessary. The location anchored at the inner membrane is especially critical for the unexpected effect of efficient enhancement of the activation signal. Unexpectedly, the second co-stimulatory signal and the third signal CD3zeta are also important for efficient triggering of the CAR signals. Surprisingly, the SAP or EAT2 signal booster lead to the unexpected effect of significantly enhancing the NK cells activation and helping to overcome the HLA-1 stimulated inhibitory receptor signaling barrier. By employing the complete signaling designs, we identified a series of excellent CAR candidates, which possess the unexpected effect of efficiently activating NK cell functions and displaying complete resistance to the inhibition receptor signaling.

### 6.2 Example 2: Exemplary Materials and Methods

### 6.2.1 Cells

All cells were cultured at 37° C. and 5% CO₂. NK92 cells were cultured in RPMI-1640 supplemented with 2 mM L-glutamine (Sigma), 1.5 g/L sodium bicarbonate (Sigma), 0.2 mM inositol (Sigma), 0.1 mM 2-Mercaptoethanol (Sigma), 0.02 mM Folic acid (Sigma), 100 IU/mL recombinant IL-2, 12.5% horse serum (Sigma) and 12.5% Fetal Bovine Serum (FBS, Sigma). NALM6 cells were cultured in complete RPMI-1640 supplemented with 10 mM HEPES, 2 mM L-glutamine, 1×non-essential amino acids, 1 mM sodium pyruvate, 50 uM 2-mercaptoethanol and 10% FBS. 293T and HUH7 cells were cultured in complete Dulbecco's Modified Eagle Medium DMEM with 10% FBS.

Human primary NK cells were isolated from healthy human donors' peripheral blood using NK cell negative selection kit (STEMCELL Technoloties Inc.), Freshly isolated NK cells were cultured with Paraformaldehyde (PFA) fixed K562-CD137L cells (1:1) with 100 IU/mL IL-2 at 37° C. and 5% CO₂ in serum-free OpTmizer T Cell Expansion medium (Gibco) with 10% of human IL-2 preparation (Hemagen Diagnostics). NK cells were used to perform gene modification at day 3. And the cultured NK cells were used 2 weeks after the gene modification.

### 6.2.2 Vector Constructs

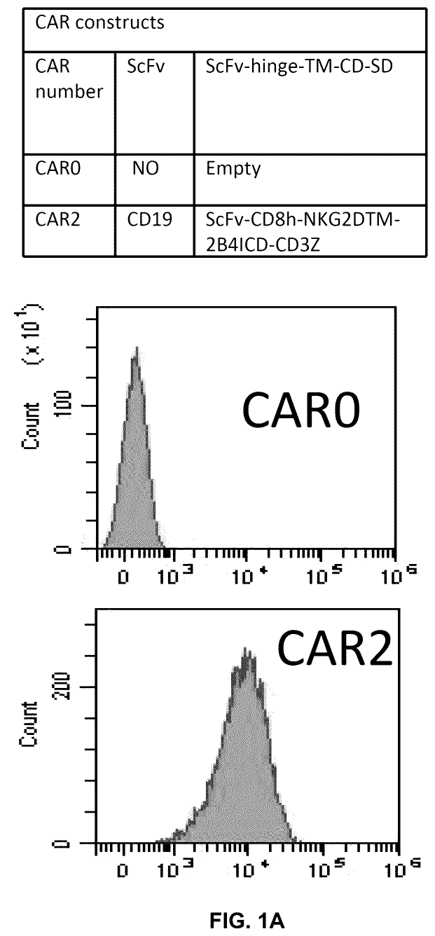

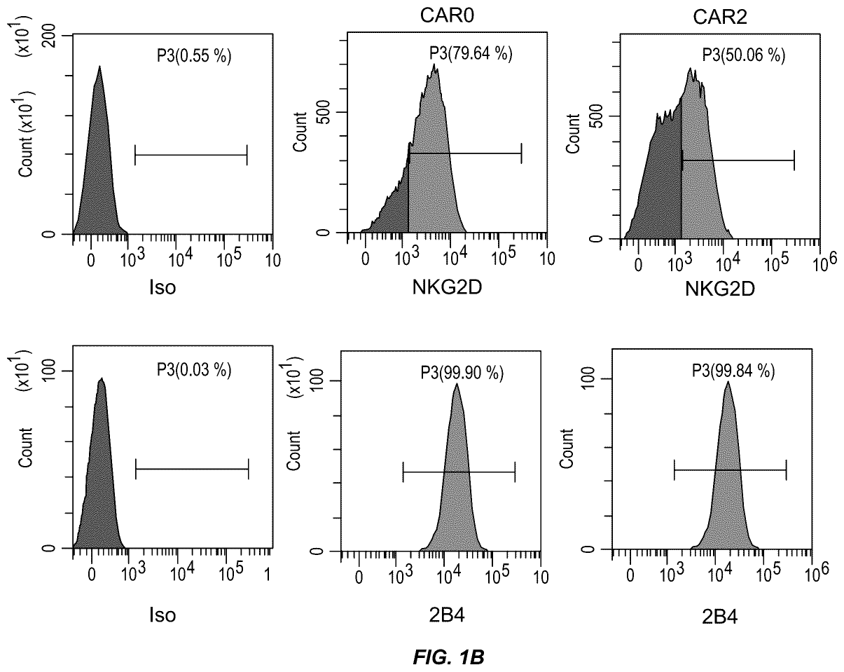

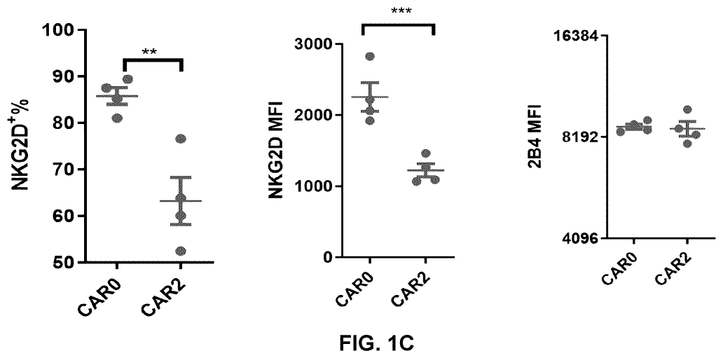

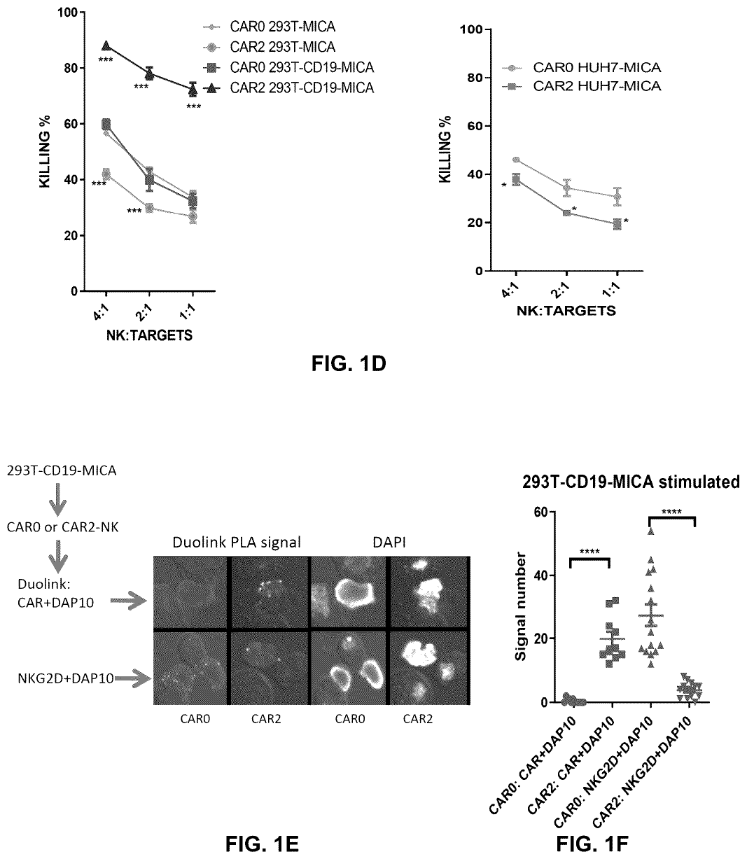

Primers and long DNA fragments were synthesized by ThermoFisher Scientific, CAR constructs were cloned into BglII/XhoI of MSCV retrovirus vectors. CAR backbone with ScFv recognizing CD19 or GPC3 were generated based on the designs shown in FIG. 1-7, and the gene sequences used in experiments shown in Table 4. The P2A sequence used for SAP expression following CAR is GSGATNFSLLKQAGDVEENPGP (SEQ ID NO: 1).

TABLE 4
Gene sequences used in experiments

| | NCBI | Signal | | | |
|---|---|---|---|---|---|
| Gene | Reference | peptide | Hinge | TM | |
| name | Sequence | (nt) | (nt) | (nt) | ICD(nt) |
| CD8 | NM_001768.7 | 89-151 | 500-634 | | |
| CD28 | NM 006139.4 | | 455-514 | 515-595 | 596-718 |
| NKG2D | NM_007360.4 | | | 318-380 | |
| CD3zeta | NM_198053.3 | | | | 218-559 |
| | | | | | (including stop) |
| DAP10 | NM_014266.4 | | | | 273-344 |
| 2B4 | NM_001166663.2 | | 592-897 | 898-960 | 961-1320 |
| CD229 | NM_002348.4 | | 1308-1376 | 1377-1442 | 1443-1979 |
| NTB-A | NM_001184714.2 | | 442-741 | 742-804 | 805-1059 |
| CRACC | NM_021181.5 | | 431-733 | 734-796 | 797-1060 |
| CD84 | NM_001184879.2 | | 651-734 | 735-797 | 798-1094 |
| CD2 | NM_001767.5 | | 424-690 | 691-768 | 769-1116 |
| CD28H | NM_144615.3 | | 347-454 | 455-517 | 518-850 |
| DNAM1 | NM_006566.4 | | 793-834 | 835-897 | 898-1080 |
| SAP | NM_002351.5 | | | | |
| EAT2 | NM_053282.5 | | | | |
| MICA | NM_000247.3 | | | | |
| MICB | NM_005931.5 | | | | |
| HLA-E | NM_005516.6 | | | | |
| CD48 | NM_001256030.2 | | | | |
| GPC3 | NM_001164617.2 | | | | |
| CD19 | NM_001178098.2 | | | | |
| CD137L | NM_003811.4 | | | | |
| LUC2 | MK484108.1 | | | | |

### 6.2.3 In Vitro Killing, Degranulation Assays

293T, 293T-CD19, NALM6, and Huh7 target cells were genetically modified and stably expressed Luc2-GFP as described previously (Zhang et al. (2021) Ectopic activation of the miR-200c-EpCAM axis enhances antitumor T cell responses in models of adoptive cell therapy. Sci Transl Med. 13(611):eabg4328). To assess killing activities, CAR-NK cells were co-incubated with target cells at 37° C. with different ratios for different time as indicated in figure legends 0.75 mg/mL D-Luciferin was used to measure luminescence emission spectra. Target cells without NK cells were used as 100% live cell control. The killing ability was determined based on the normalized spectrum and background subtraction and calculated using the equation killing %=(1−(exp/100% live control))*100 (Zhang et al. (2021) Ectopic activation of the miR-200c-EpCAM axis enhances antitumor T cell responses in models of adoptive cell therapy. Sci Transl Med. 13(611):eabg4328).

To assess degranulation, 2 g/ml fluorescently conjugated anti-CD107a antibody was added to the NK-target cocultures and incubated at 37° C. for 2 hours, cells were then washed with FACS buffer including 1 mM EDTA, and stained with anti-CD56 antibodies. CD56 stained CD107a+NK cells was quantified by flow cytometry analysis (Beckman Coulter CytoFLEX LX).

### 6.2.4 Flow Cytometry Analysis of Intracellular Cytokines

To assess IFN production, live NK cells were co-incubated with target cells (1:1) in U-bottom 96-well plates after spinning at 300 g for 3 minutes and incubated at 37° C. for 2 hours. Brefeldin A was added to the cells at final concentration of 5 μg/ml and incubated at 37° C. for additional 4 hours. Cell surface markers were stained, washed, and fixed with IC Fixation Buffer (eBioscience), and washed and stained with cytokine antibodies using permeabilization buffer (eBioscience), and then analyzed by flow cytometry.

### 6.2.5 Duolink PLA

PLA assays were performed using the in situ Duolink PLA red kit (Sigma-Aldrich) according to the manufacturer's instructions. The CAR0, an empty vector with GFP expression, or CAR2-GFP fusion expressing NK cells were attached to the poly-l-lysine glass, target cells were then added and incubated at 37° C. for 30 minutes. Unattached cells were removed by washing with PBS. slides were fixed and stained with anti-GFP (Rabbit IgG) and anti-DAP10 (Mouse IgG), washed, and stained with anti-rabbit and anti-mouse PLA probes, followed by probe DNA ligation, amplification, Fluorescent staining were then performed and observed using confocal microscopy,

### 6.2.6 Retroviral Transduction

For NK cell transduction, Phoenix A cells were transfected with expression vectors together with the packaging vector GagPol using the calcium method. 0.45 μm filtered viral supernatant, 8 μg/ml polybrene and IL-2 (100 IU/mL) were mixed with NK line cells or 3-day expanded primary NK cells in 6-well plates. The plates were centrifuged at 1400×g for 2 hours at 35° C. The transduced NK line cells were cultured with respective media for in vitro experiments, the transduced primary NK cells were co-cultured with inactivated K562-CD137L and recombinant IL-2 (100 IU/mL) for 14 days and used for in vitro validation and adoptive cell therapy experiment in mice in the next step.

### 6.3 Example 3: Dysfunction of Natural NKG2D in NKG2D-CAR NK Cells

NKG2D is a activation receptor expressed by NK cells, it alone cannot trigger NK cells activation (Long et al. (2013) Controlling natural killer cell responses: integration of signals for activation and inhibition. Annu Rev Immunol. 31:227-58). Tumor cells usually express various ligands for NK activating receptors. For example, 293T cells not only express MICA/B (NKG2D) ligand, but also express ligand for NKp30 (Flaig et al. (2004) Cutting edge: NTB-A activates NK cells via homophilic interaction. J Immunol. 172(11):6524-7) activation of NK cells requires the synergy of multiple activation receptors.

We used empty vector as the CAR0, and the reported NKG2D CAR as CAR2 (FIG. 1A), the data showed that CAR2 NK cells express lower level of NKG2D, but the same level of 2B4 (FIGS. 1B-IC). The reported NK specific NKG2D-TM based CAR is more effective than T-CAR expressed on NK cells. But NKG2D CAR expression reduced natural NKG2D expression in NK cells as reported (Li et al. (2018) Human iPSC-Derived Natural Killer Cells Engineered with Chimeric Antigen Receptors Enhance Anti-tumor Activity. Cell Stem Cell. 23(2):181-192.e5), indicating that the NKG2D CAR expression may impair the function of natural NKG2D.

In order to observe the function of constitutive natural NKG2D, we used different target cells, such as 293T cells and HUH7-MICA cells. 293T (no CD 19 expression) cells express NKG2D ligands such as MICA/MICB. HUH7 (no CD 19 expression) cells have no constitutive MICA/MICB expression, and a MICA/MICB stable expression HUH7 cell line was established.

Targeting to 293T-CD19-MICA cells, CAR2-NK cells showed excellent killing ability (FIG. 1D). When using 293T-MICA, although there is no CD19 expression on the target cells, CAR0-NK and CAR2-NK cells could kill them in a lower level, and the CAR2 expression on NK cells reduced the killing trigger than CAR0. The same was observed when using Huh7-MICA as target cells (FIG. 1D). This is at least partially due to the lower NKG2D expression in CAR2 NK cells, and also related to the signaling adaptor occupied by NKG2D-CAR as shown in FIGS. 1E-1F.

NKG2D receptor has no intracellular signaling domain, it recruits adaptor DNAX-Activation Protein 10 (DAP10) to transfer signals via YxxM motif (Eagle et al. (2009) Beyond Stressed Self: Evidence for NKG2D Ligand Expression on Healthy Cells. Curr Immunol Rev. 5(1):22-34). Duolink proximity ligation assay (PLA) is a technique to observe molecule-molecule interactions, signal is displayed when the distance is less than 30 nm between two molecules. We used CAR0 NK cells expressing GFP as control, and used CAR2-GFP fusion molecule expression NK cells to co-incubate with target 293T-CD19-MICA cells, PLA was performed between GFP and DAP10. DAP10 was recruited to the CAR2 effectively stimulating by CD19. When using NKG2D-GFP expression NK cells, the PLA between GFP and DAP10 showed CAR2 expression reduced the recruitment of DAP10 to the natural NKG2D significantly (FIGS. 1E and 1F). The data indicate that CAR2 recruits DAP10 to transfer signals and trigger NK cell activation, and at the same time, they occupy the resource of DAP10, leading to the lack of the DAP10 for natural NKG2D activation.

FIGS. 1A-1E demonstrate the following results: 1. NKG2D-CAR-NK cells express lower level of NKG2D, but same level of 2B4. 2. Lower natural NKG2D function of NKG2D-CAR-NK cells. 3. Lower interaction of NKG2D with DAP10 in NKG2D-CAR NK cells.

### 6.4 Example 4: Complete Signaling 2B4-CAR Mediated Anti-Tumor Activity of NK Cells

Without being bound by the theory, we designed novel complete signaling CARs for NK cells based on NK cell biology. 2B4 (CD244) is an activation receptor expressed by NK cells (Agresta et al. (2018) The Emerging Role of CD244 Signaling in Immune Cells of the Tumor Microenvironment. Front Immunol. 9:2809), which belongs to signaling lymphocyte activation molecule family (SLAMF4). Without being bound by the theory, the natural ligand of 2B4 has been identified as CD48. Without being bound by the theory, the cytoplasmic tail of 2B4 contains four Immunoreceptor Tyrosine-based Switch Motifs (ITSMs). Without being bound by the theory, the ITSMs can recruit Signaling Lymphocyte Activation Molecule (SLAM)-associated protein (SAP), which is associated with activation signaling via competitively binding to ITSMs and inhibiting the SHP recruitment. Without being bound by the theory, ITSMs can also recruit Ewing sarcoma-activated transcript 2 (EAT2), which is associated with activation and inhibition signaling. Without being bound by the theory, SAP can: bind to both non-phosphorylated and phosphorylated ITSMs, but EAT2 can only bind to phosphorylated SLAMF ITSMs (Morra et al. (2001) Structural basis for the interaction of the free SH2 domain EAT-2 with SLAM receptors in hematopoietic cells. EMBO J. 20(21):5840-52), which may limit the effect of EAT2 in 2B4 signaling by only partially inhibiting the binding of SHP to ITSMs. Without being bound by the theory, SAP and EAT2 have two functions: 1) help to activate Fyn in SLAMF signaling, 2) to block the binding of SHP to the pTyr in ITSMs of 2B4, which eliminates the inhibition by SHP molecules.

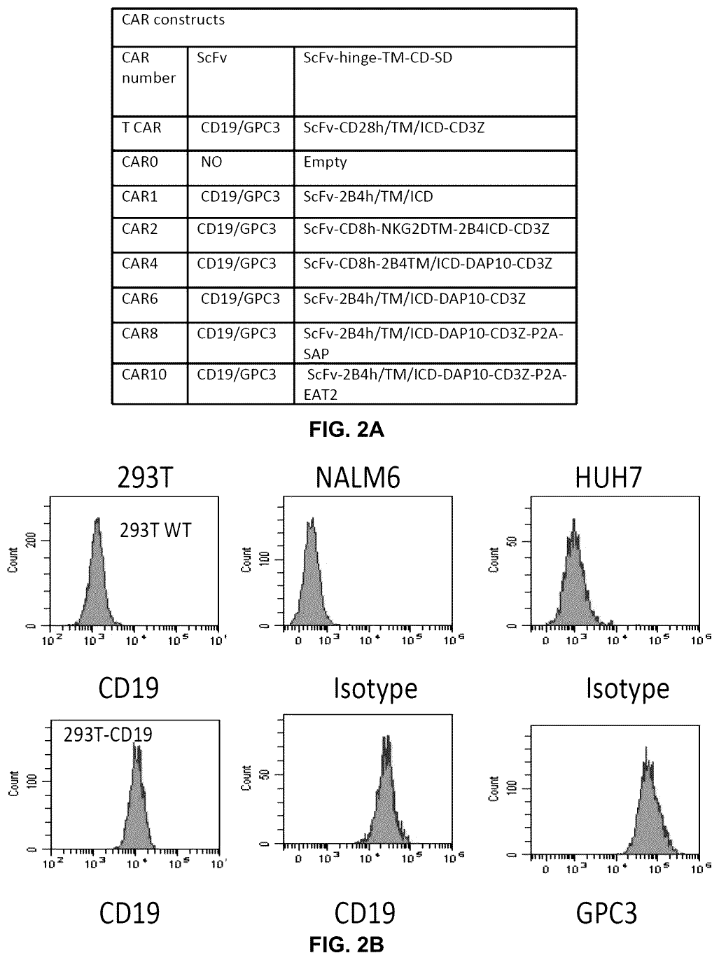

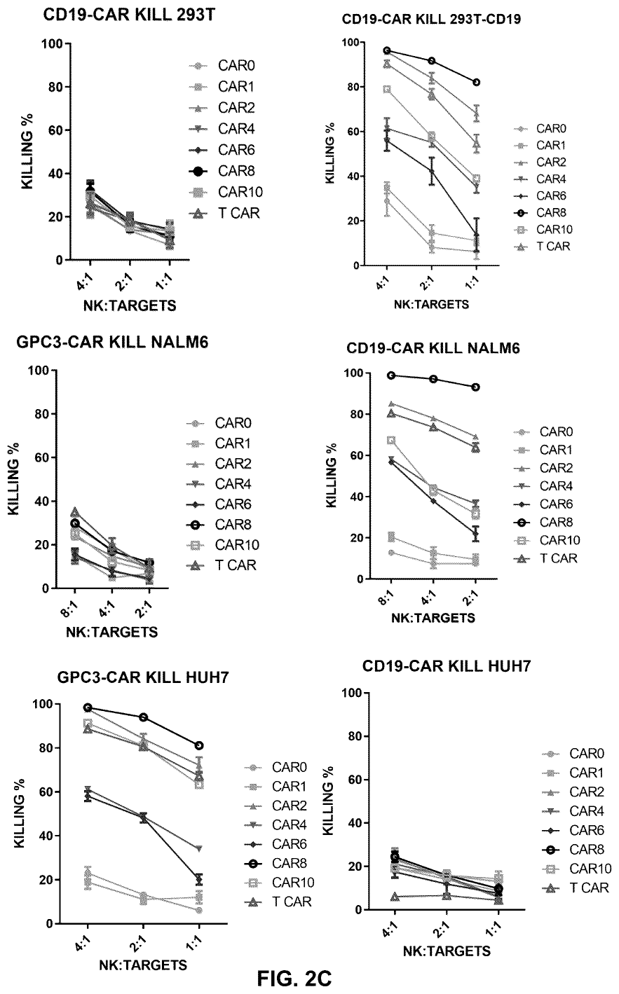

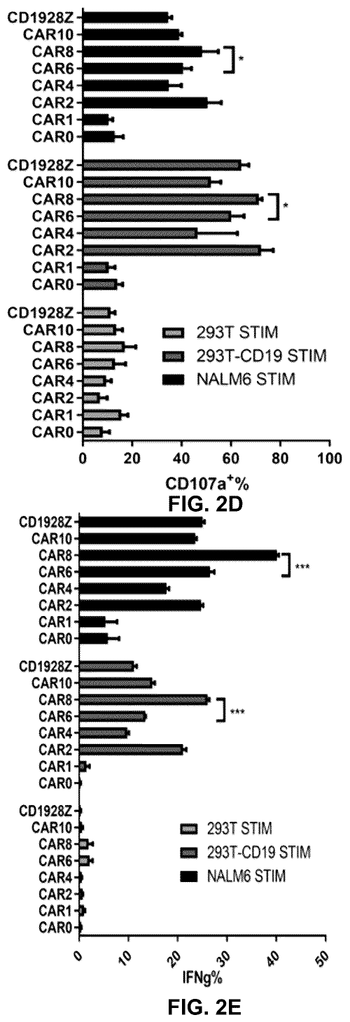

We designed the novel CAR as shown in FIG. 2A. The 2B4 hinge TM, the DAP10 ICD and CD3zeta ICD were used to generate the novel effective NK CAR. Without being bound by the theory, since 2B4 exhibits both inhibitory and activating functions, the NK CAR was designed to have a SAP signal or EAT2 signal directed by the P2A following the CAR construct. After translation, the P2A is cut by enzymes to release the CAR and the SAP or EAT2 protein (Liu et al. (2017) Systematic comparison of 2A peptides for cloning multi-genes in a polycistronic vector. Sci Rep. 7(1):2193). Without being bound by the theory, the SAP or EAT2 binds the ITSMs at the tail of 2B4 and competes with the inhibitory signal molecule SHP. So the complete activation signaling can be achieved in the NK-CAR. Data showed that, unexpectedly, all of the designs triggered activation signaling successfully compared to empty vector (CAR0), and control CAR1 without intracellular signaling DAP10 and CD3zeta (FIGS. 2C-E).

Hinge is reported to be critical for the effective CAR expression and function, we compared to use 2B4 hinge and CD8 hinge, both could trigger the CAR to be activated effectively in NK cells, suggesting that the 2B4 hinge can be used in NK-CAR to provide an effective activation signal for NK cells (FIGS. 2C-E).

Inhibitory receptors, such as NKG2A, can bind to and triggered by HLA-I molecule HLA-E, results in the triggering of the inhibitory molecule SHP. 2B4 can bind to SHP to inhibit the receptor signaling, and SAP or EAT2 can bind to the tail of 2B4 to compete with SHP, leading to a net activation signaling. Surprisingly, data showed that SAP (in CAR8) and EAT2 (in CAR10) can be used as effective signaling boosters to generate the complete signaling to activate the CAR-NK (FIGS. 2C-E). SAP functions better than EAT2 in this setting.

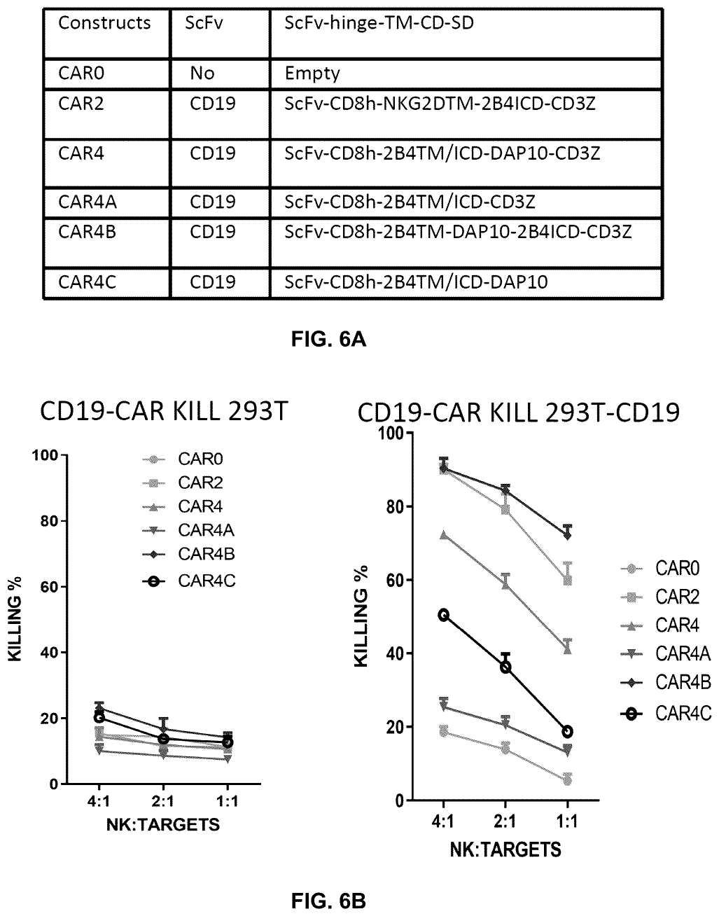

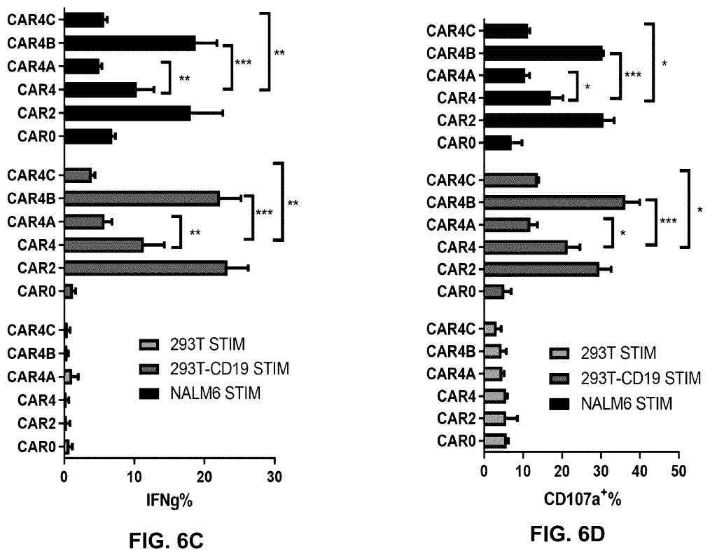

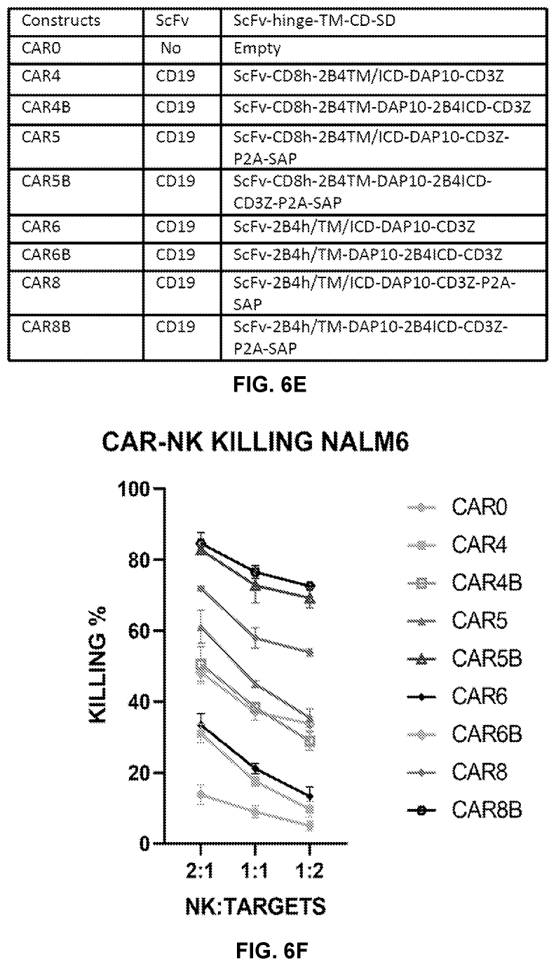

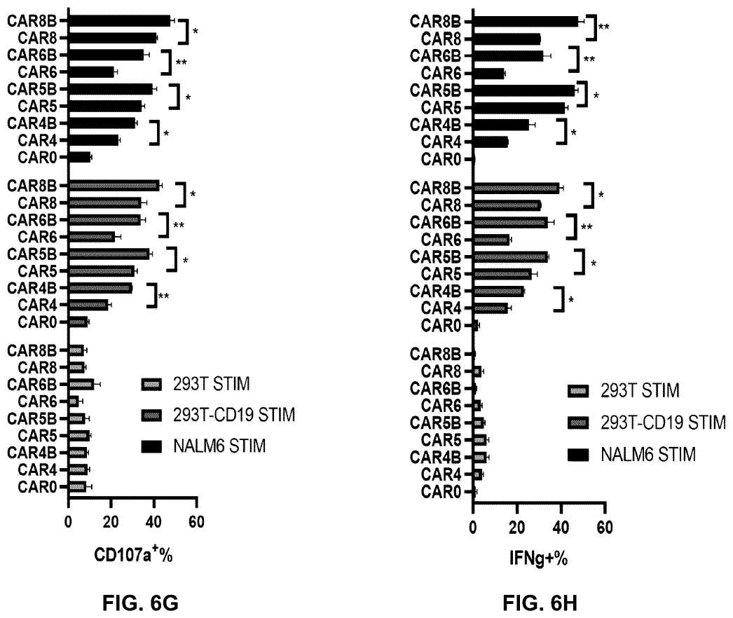

In addition, CAR4 and CAR6, that is CD8hinge-2B4-TM/ICD CAR and 2B4-hinge/TM/ICD CARs, both are effective in triggering activation signals, but is still limited without the fourth SAP signal (FIGS. 2C-E), which is discussed later as in FIG. 6.

FIGS. 2A-2E demonstrate the following results: 1. 2B4 can be used in TM, ICD to make effective NK CAR. 2. 2B4 hinge can be used as effective one in the CAR construct. 3. Surprising, DAP10 used to replace the NKG2D is good in CAR construct. 4. SAP and EAT2 unexpectedly boost the NK activation signaling, which make up the complete signaling NK-CAR (NK-csCAR), and SAP can be better than EAT2. 5. CAR4 and CAR6, that is CD8hinge-2B4-TM/ICD CAR and 2B4-hinge/TM/ICD CAR are less effective than NKG2D-CAR (FIG. 6).

Data above indicating that our novel design works surprisingly well, and complete signaling CAR can trigger NK cell activation effectively in an unexpected level.

### 6.5 Example 5: SAP Boosted Complete Signaling

As shown in FIG. 2, 2B4 CAR can successfully trigger the NK cell activation (CAR6), and SAP, as a complete signal booster, can efficiently block the inhibitory receptor signaling barrier mediated by SHP (in CAR8).

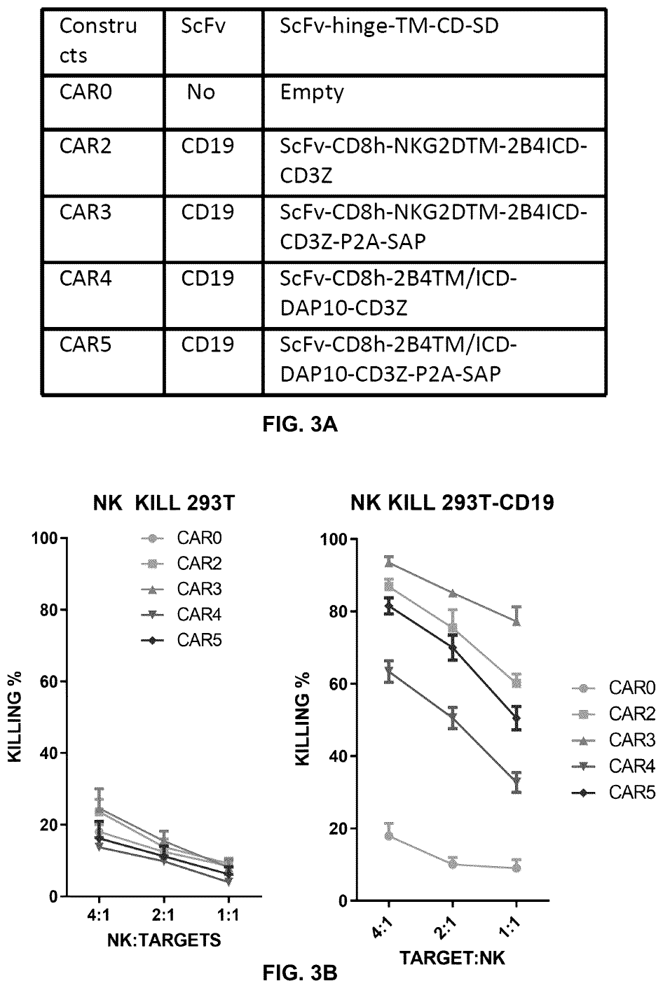

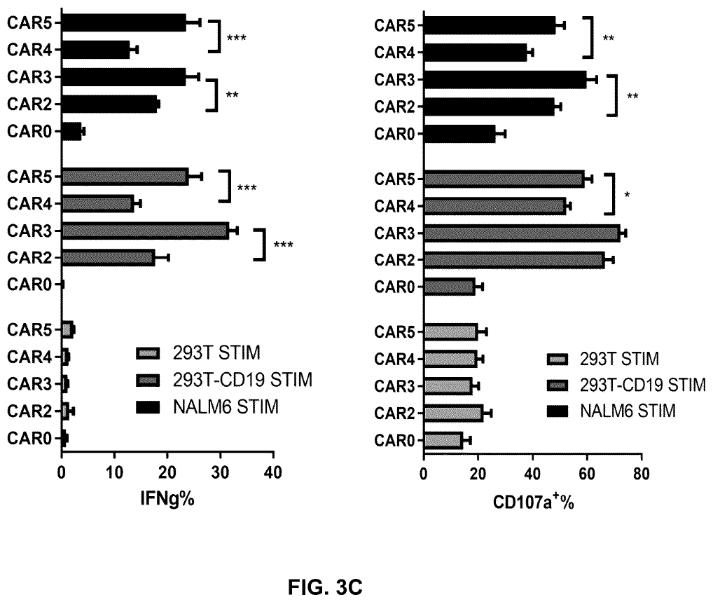

To investigate the effects of using the fourth signal SAP and using the CD8 hinge instead of 2B4 hinge in CAR4, new constructs, CAR3 and CAR5, were designed by adding the SAP signal booster in CAR2 and CAR4. (FIG. 3A). Similar from CAR6 to CAR8 by adding SAP (FIG. 2), the addition of SAP to make CAR3 from CAR2, and CAR5 from CAR4, had the same effect, the signal booster SAP effectively enhanced the signaling in CAR3 and CAR5. The killing ability, degranulation and IFNg production of CAR3-NK cells was significantly increased compared to that of CAR2-NK cells, and that of CAR5-NK cells was also improved compared to that of CAR4-NK cells (FIGS. 3B and 3C). Although the function of CAR5-NK cells is similar with CAR2-NK cells, the function of CAR4-NK cells were still limited, which is the same phenomenon shown in FIG. 2, and is discussed later as in FIG. 6.

FIGS. 3A-3C demonstrate the following results: 1. Unexpectedly, SAP can boost the killing ability of CAR-NK cells. 2. Unexpectedly, SAP can give the complete signaling to CAR-NK cells to stimulate degranulation and IFNg production. 3. Still, 2B4 CAR is less effective than NKG2D CAR, explained in FIG. 6.

The data above indicating that SAP boosted CARs provide complete signaling in NK cells and improve the function of NK cells effectively.

### 6.6 Example 6: 2B4-CAR-NK Cells have Normal Natural NKG2D Function, and Complete Signaling CAR Boosts the Natural 2B4 Signaling

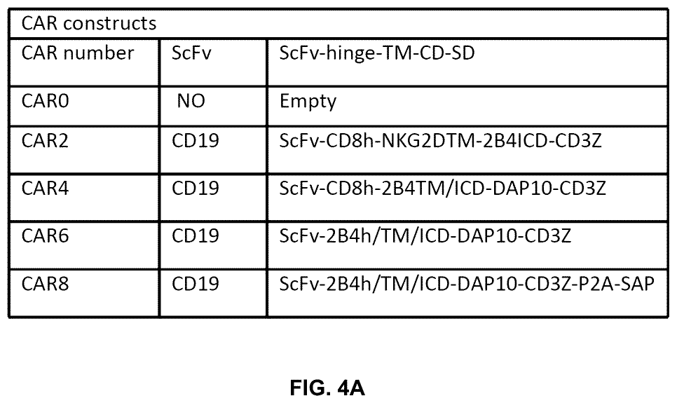

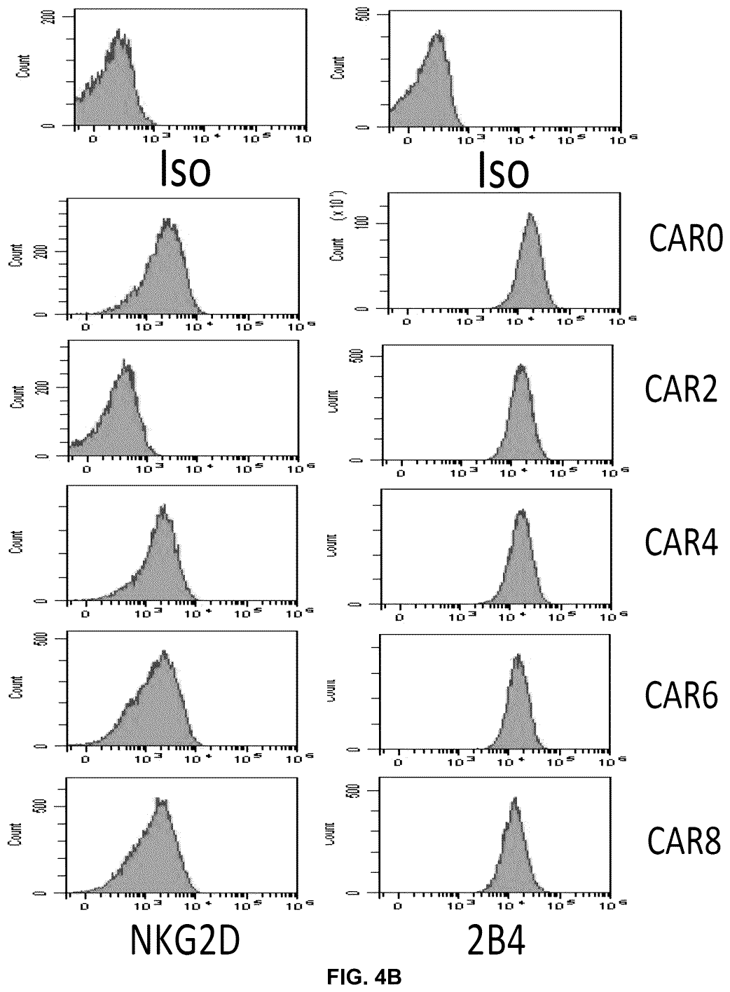

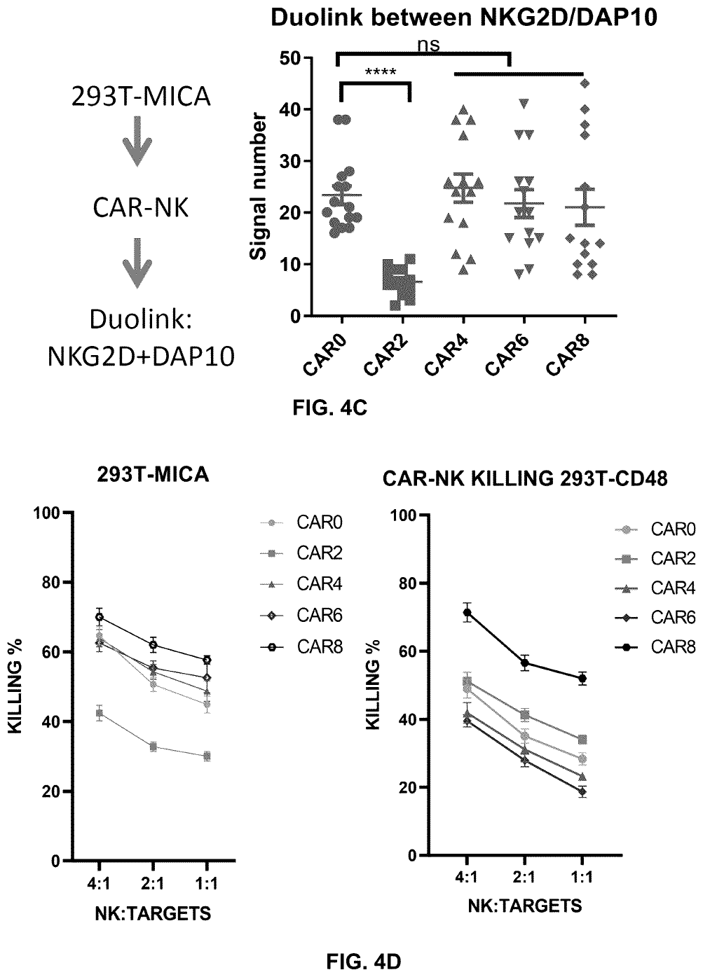

As shown in FIG. 1, we confirmed that the NKG2D CAR (CAR2) NK cells have reduced constitutive natural NKG2D expression and dysfunctional NKG2D. In order to determine if there is the same phenomenon in our novel designed CAR NK cells, we compare the NKG2D function of CAR2, CAR4, CAR6 and CAR8 (FIG. 4A). In CAR expressed NK cells, the NKG2D expression was reduced in CAR2 expressed NK cells, but it was normal in other CAR-NK cells, also, the 2B4 expression is similar between these several CAR-NK cells (FIG. 4B).

The duolink PLA assay between NKG2D and DAP10 showed that unlike the CAR2-NK cells, the several novel designed CAR-NK cells all have the normal recruitment of DAP10 to NKG2D receptors (FIG. 4C), indicating the normal signaling of NKG2D receptors in these novel designed CAR-NK cells. Consistently, NKG2D mediated 293T-MICA cell killing showed CAR2 reduced and CAR4, CAR6, CAR8 improved the killing process, and in 293T-CD48 cell killing, although the synergy of NKG2D and 2B4 receptors, the killing percentage was not increased except for the CAR8 NK cells, indicating a similar 2B4 function except for CAR8 enhancing the signaling significantly (FIG. 4D). Which may be due to the two directions of 2B4 receptor signaling, the inhibitory polarization signaling limited the NK cell activation.

FIGS. 4A-4D demonstrate the following results: 1. NKG2D CAR is dysfunctional of natural NKG2D, with lower NKG2D-DAP10 binding. 2. 2B4-DAP10-CD3Z CAR-NK cells have normal NKG2D function. 3. 2B4 expression is normal in CAR-NK cells, and 2B4 has two direct functions. 4. Unexpectedly, SAP complete signaling CAR has boosted activation and killing ability.

Altogether, our newly designed 2B4 CARs have normal and effective natural NKG2D receptor function, and the newly designed 2B4 CARs, especially the CAR8 with SAP boosted complete signaling NK-CARs are able to maintain the multifunctional properties of the NK cells during CAR-NK cell therapy.

### 6.7 Example 7: Complete Signaling CAR (csCAR) can Effectively Overcome HLA-I Inhibition Barrier

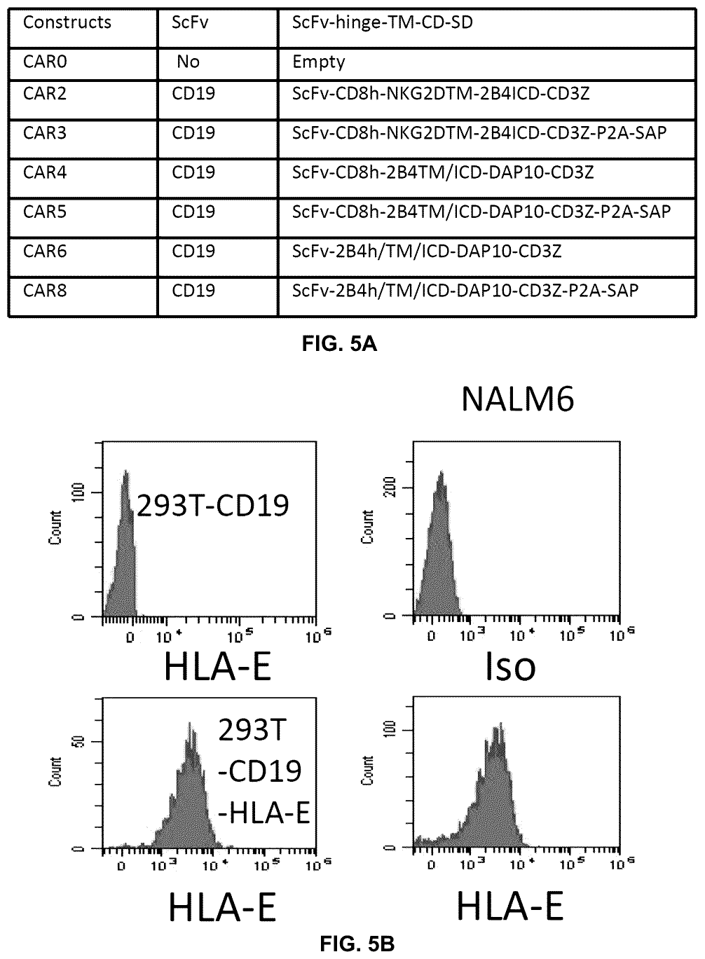

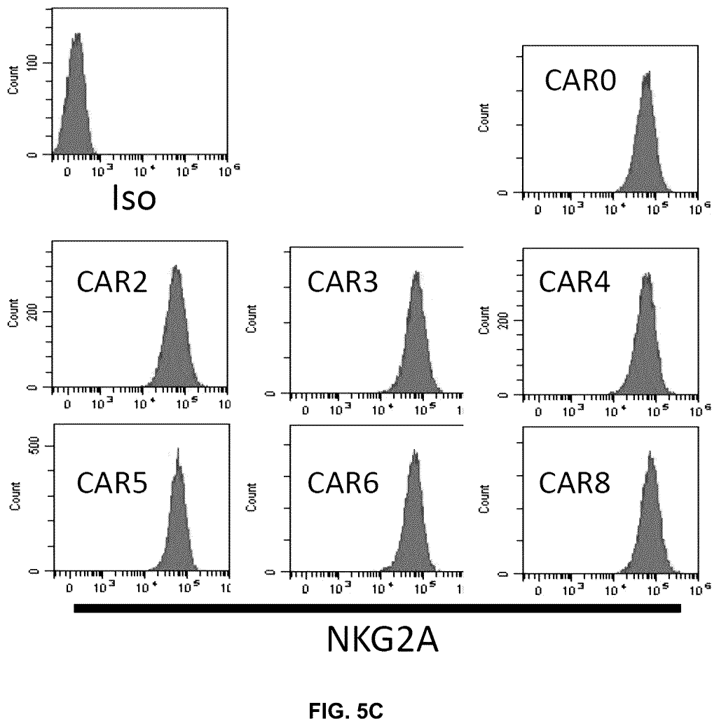

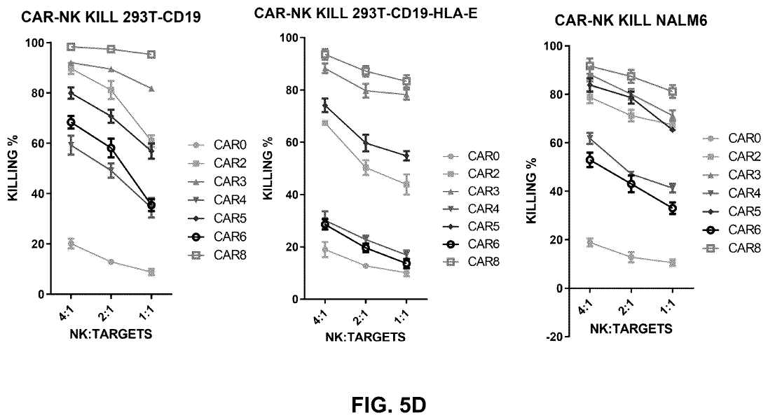

Without being bound by any particular theory, HLA class I (HLA-I) endows NK cells with inhibitory response and facilitates the process of licensing or education, but in tumor immunotherapy, the adoptively transferred NK cells have to overcome the inhibitory signaling barriers from HLA-I expressed on tumor cells (Long et al. (2013) Controlling natural killer cell responses: integration of signals for activation and inhibition. Annu Rev Immunol. 31:227-58). Without being bound by any particular theory, the inhibitory receptors of NK cells usually use SHP to inhibit vav1 function and phosphorylate CSK to affect the cytoskeleton organization. Without being bound by any particular theory, SAP competes with SHP to bind pTry at ITSM tail of SLAMF, which blocks the SHP binding and removes the inhibitory potential of HLA-I. In complete signaling CAR (csCAR) constructs (FIG. 5A), SAP is expressed with the CARs (in CAR3, CAR5, and CAR8) and we then determined if the CARs can overcome the inhibitory signals when triggered by the antigen on tumor cells.

Without being bound by any particular theory, HLA-E (Major Histocompatibility Complex, Class I, E) belongs to the HLA class I heavy chain paralogues, and it is a major ligand for the natural killer inhibitory receptor CD94/NKG2A. Without being bound by any particular theory, NKG2A is an inhibitory receptor expressed by NK cells, which binds to HLA-E and triggers inhibitory signaling dependent on the recruitment of the SHP. We established stable expression of HLA-E on 293T-CD19 cells, and NALM6 cells constitutively express HLA-E (FIG. 5B). Although NKG2D expression was reduced in CAR2 NK cells (FIG. 4B), there was no obvious difference of NKG2A expression between different CAR-NK cells (FIG. 5C). Nevertheless, HLA-E and NKG2A interaction provides inhibitory signaling, which reduced the killing ability of NK cells, especially for NK cells with CAR2, CAR4 or CAR6 (FIG. 5D).

Surprisingly, SAP boosted CAR provided the complete signaling and overcame HLA-E inhibitory barrier effectively in CAR3, CAR5 and CAR8 NK cells (FIG. 5D). Here, the CAR3 is from CAR2 (NKG2D CAR) with the addition of P2A-SAP, CAR5 from CAR4 with the addition of P2A-SAP, CAR8 from CAR6 with the addition of P2A-SAP. The data suggested that SAP boosted CAR enhanced the killing ability and was able to overcome the inhibitory signaling barrier from HLA-I triggered inhibitory receptor.

After several weeks of culture using feeder cells, unexpectedly, CAR and SAP gene modified NK cells maintained very high functions after several weeks culture with feeder cells (FIG. 5D), suggesting that NK cell inhibition would be only removed when triggered by tumor antigens, and that the normal licensing process would be maintained without tumor antigen and meet with HLA-I on healthy cells. So that from the culture enhancement process, survival and licensing process during in vitro culture and after injection into the vein, and also the super effective function of NK cells meeting the antigen expressing tumor cells, indicating that our novel NK csCAR designs is a very potent approach for the cancer immunotherapy.

FIGS. 5A-5D demonstrate the following results: 1. NKG2A expression is the same in different CAR-NK cells. 2. HLA-E and NKG2A interaction provides inhibitory signaling. 3. SAP boosted CAR complete signaling can overcome HLA-E barrier effectively.

### 6.8 Example 8: Signaling Domains of NK-CAR in NK Signaling, DAP10 and CD3Zeta are Necessary, and DAP10 Anchored at the Inner-Membrane is Critical to Enhance Activation and Killing Activities of NK Cells

As shown in FIG. 2 and FIG. 3, although CAR4 and CAR6 could effectively trigger the activation signaling, the efficiency was limited without the fourth signal SAP. Theoretically, the signals should be sufficient even without the SAP, at least the efficiency should be the level as CAR2. As we know, the NKG2D has no signal tail, but recruit the DAP10 adaptor to deliver signals, and DAP10 is a transmembrane protein with a YxxM motif in its tail. Through the YxxM, DAP10 can activate phosphatidylinositol 3-kinase (PI3K) dependent signaling pathways, leading to cell survival and proliferation, and the PI3K is a group of plasma membrane-associated lipid kinases, which works on the membrane regions (Yang et al. (2019) Targeting PI3K in cancer: mechanisms and advances in clinical trials. Mol Cancer. 18(1):26; Chen et al. (2020) Research Progress on NK Cell Receptors and Their Signaling Pathways. Mediators Inflamm. 2020:6437057). In CAR4 and CAR6, the DAP10 follows the 2B4 ICD, which makes it far from the plasma membrane, therefore, may limit its function.

Using CAR4 as a model, we designed CARs with or without CD3Zeta or DAP10, and also changed the location of DAP10, to let it anchor in the inner plasma membrane or not (FIG. 6A). When NK cells expressing these CARs were compared, we found that CD3zeta and DAP10 are necessary for the CAR activation (FIGS. 6B-D). And also, surprisingly, DAP10 is location sensitive. DAP10 anchored at the inner plasma membrane is critical to strongly enhance the activation and killing ability (FIGS. 6B-D). Naturally, the DAP10 recruits to NKG2D by NKG2D transmembrane domain to trigger GRB2 and vav1 function. DAP10 anchored at the membrane uses the YxxM to activate PI3K. Without being bound by any particular theory, when DAP10 is located behind the tail of 2B4 ICD, it may be too far from the membrane to play the role, therefore, its effect to activate PI3K is reduced. Since PI3K uses the ATP and PtdIns(4,5)P2 (phosphatidylinositol 4,5-bisphosphate) to generate phosphatidylinositol 3,4,5-trisphosphate (PIP3) at the site of intracellular inner membrane, we adjusted the position of DAP10 to let it anchor at the intracellular inner membrane, making it more closer to the substrate and help it play the right role.

After discovering the critical DAP10 location in CAR4, we then compared the DAP10 location effect in CAR4, CAR5, CAR6, and CAR8 (FIG. 6E), similarly to CAR4, DAP10 anchored at the inner membrane also showed more excellent activation signaling in other CARs (FIGS. 6F-H). All data demonstrated that the DAP10 and/or CD3zeta domains are necessary for the fully CAR activation, and the activation signals in CAR NK cells were significantly improved when DAP10 was anchored at the inner plasma membrane following the TM domain. Accordingly, the most advantageous backbone of csCAR can be ScFv-SLAMF hinge-SLAMF TM-DAP10-SLAMF ICD-CD3Z-P2A-SAP.

FIGS. 6A-6H demonstrate the following results: 1. CD3Z and DAP10 are critical for the CAR activation. 2. DAP10 location is sensitive, DAP10 anchored at the inner-membrane is surprisingly critical for the good effect of enhancing activation and killing. 3. DAP10 at the inner-membrane improve CAR signaling, whatever it is with or without SAP signaling booster.

### 6.9 Example 9: Wider Screening of SLAMF and Non-SLAMF CARs in NK Cells Activation and Killing

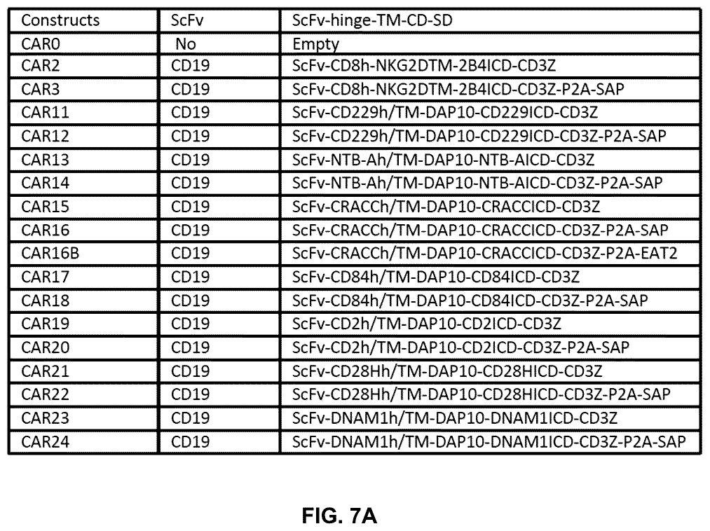

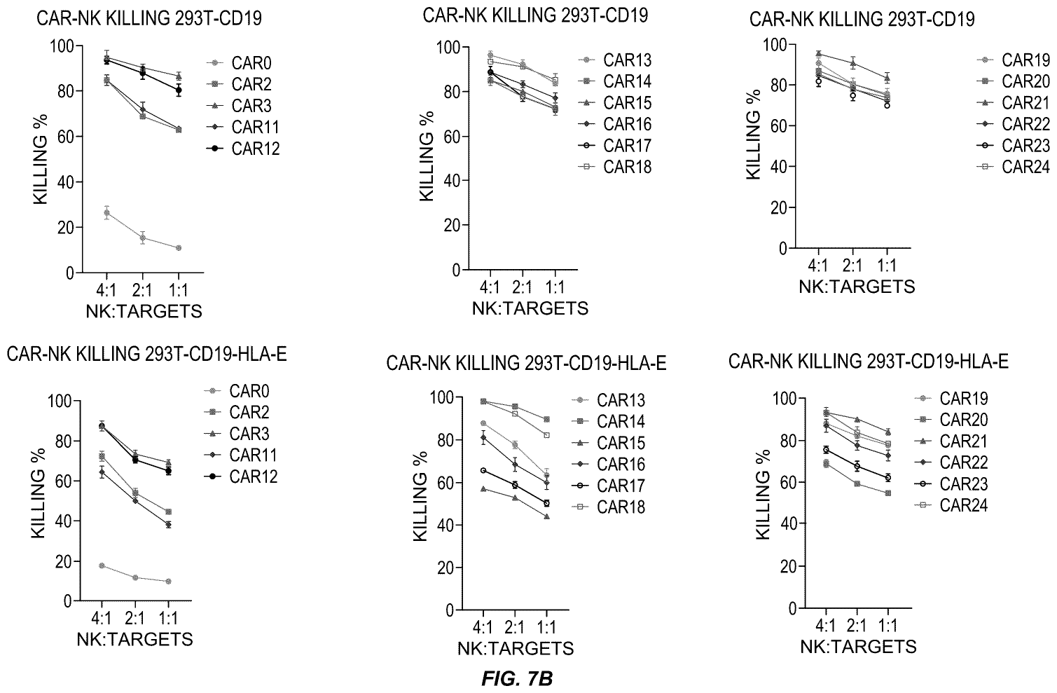

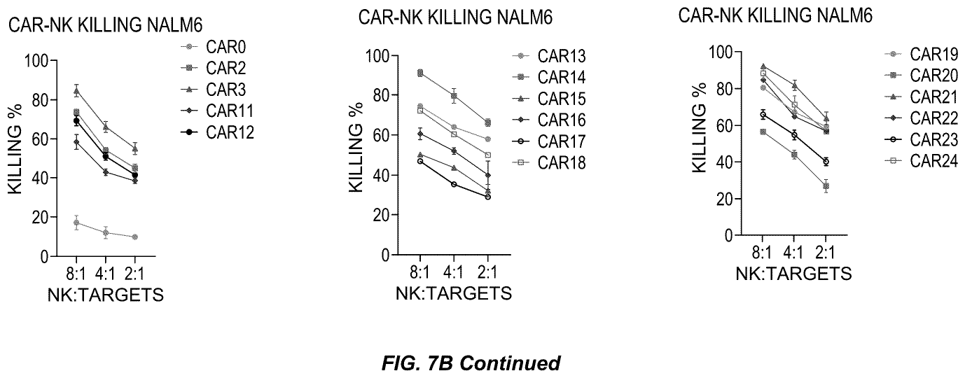

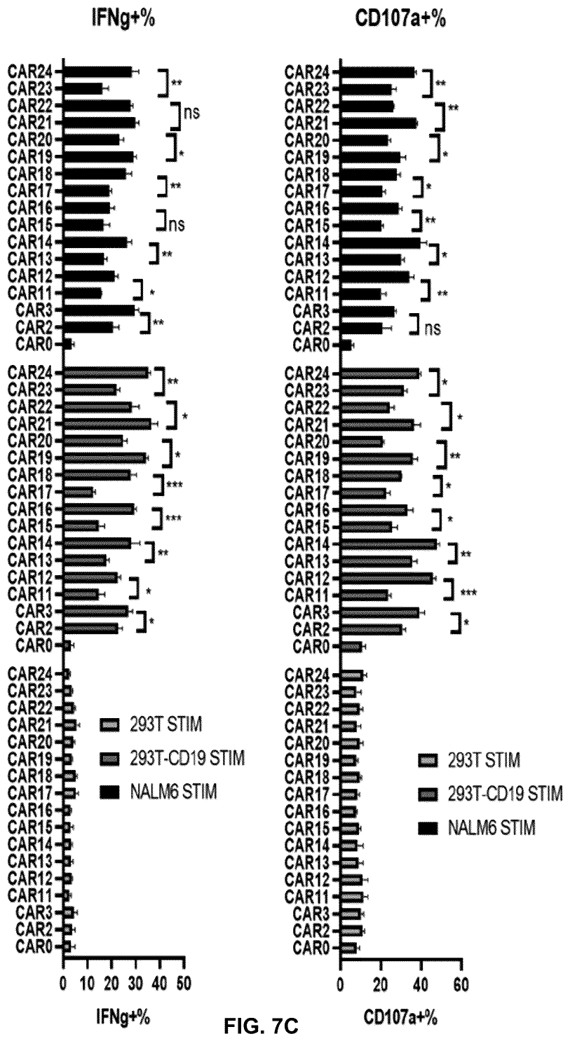

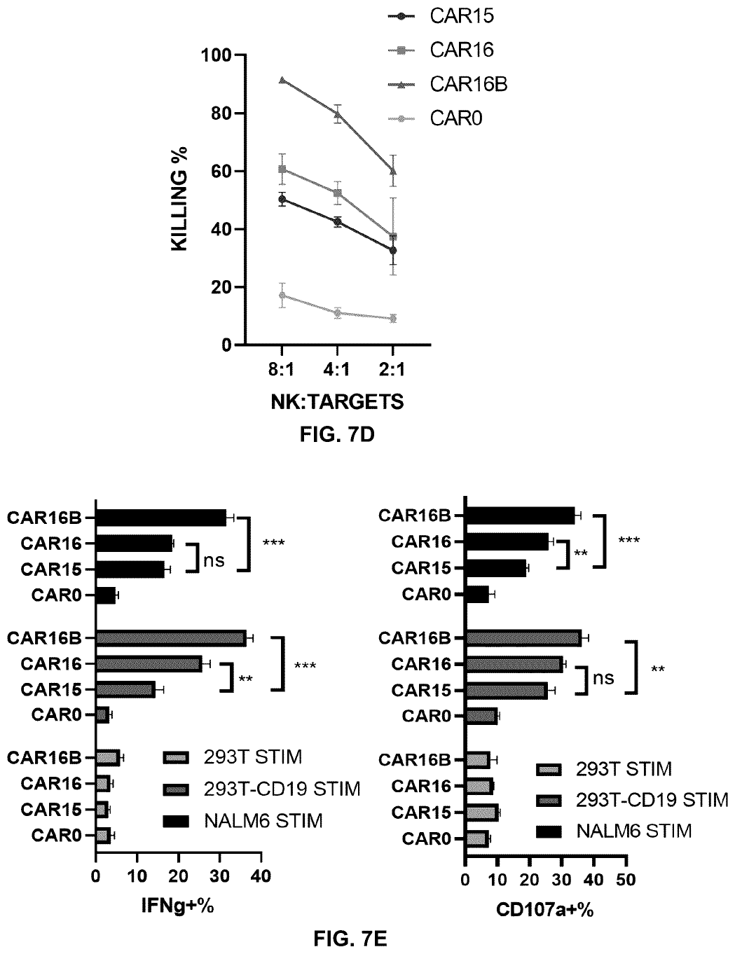

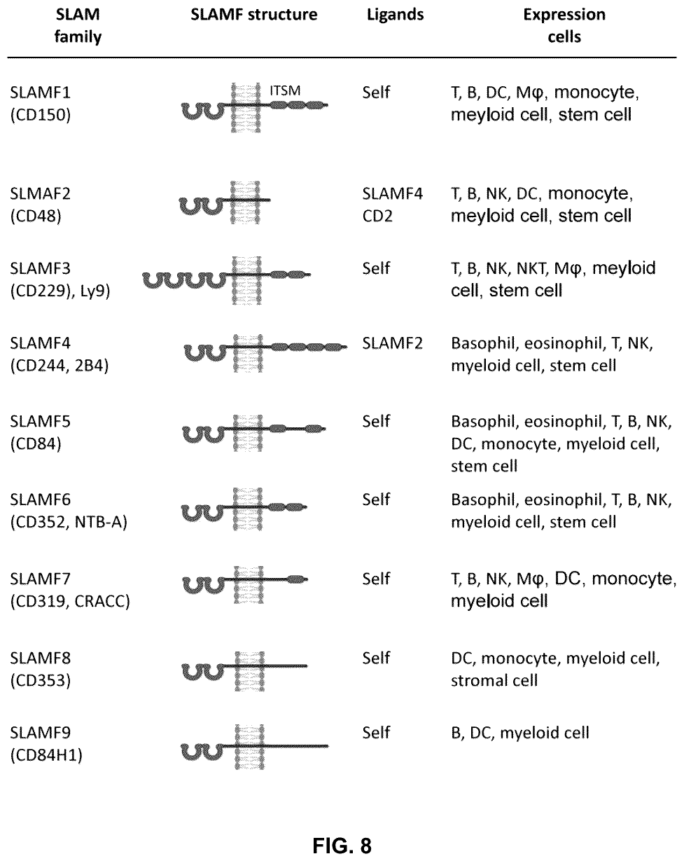

Several SLMAF family genes are expressed on NK cells, excluding SLAMF2 (CD48), which has no intracellular signaling domain (FIG. 8). Novel NK CARs using the hinge, TM and ICD of other SLAMF family genes were designed as 2B4 CAR (CAR 6 and CAR8), including CD229(CAR11-12), NTB-A(CAR13-14), CD84(CAR17-18), and CRACC(CAR15-16), and DAP10 is located in front of SLAMF ICD, that is DAP10 is anchored on the inner-membrane (FIG. 7A).

As the CAR using 2B4, CARs using CD229, NTB-A, CD84 and CRACC are effective to activate NK signaling (FIGS. 7B and 7C), and the CARs could be boosted by SAP, except for CRACC CAR, suggesting that the genes are very excellent CAR trigger, especially with the SAP as booster in CAR12,14,18 to overcome the inhibitory signaling barrier.

But in CRACC CARs (CAR15-16), HLA-E stimulated signals greatly inhibited the CAR NK function, even with SAP as a signal booster (FIGS. 7B and 7C). Reports showed that EAT2 could be recruited to the pTyr304 within the ITSM of CRACC, and EAT2 recruit and activate PLCgamma. SAP can also be recruited to the pTyr304 but at a much lower affinity. If there is lacking of EAT2, SHIP-1 can be recruited to the pTyr and mediate inhibitory signaling (Campbell et al. (2018) Mechanisms of NK Cell Activation and Clinical Activity of the Therapeutic SLAMF7 Antibody, Elotuzumab in Multiple Myeloma. Front Immunol. 9:2551). This should be why SAP cannot be as an effective signal booster for CRACC CAR. Accordingly, we designed CAR16B, using EAT2 to replace the SAP in CAR16. Data showed that EAT2 is the right signal booster for CRACC CAR (CAR16B), which could improve the NK cell activation, and also overcome the HLA-I inhibitory signaling barrier completely (FIGS. 7D and 7E).

Without being bound by any particular theory, according to the NK cell signaling property, as long as there are three synergy signals, the CAR can be activated completely. So we also designed CARs using non-SLAMF genes, such as CD2(CAR19-20), CD28H (CAR21-22) and DNAM1 (CAR23-24), In these designs, we still used the natural hinge, TM and ICD, of the CD2, CD28H or DNAM1, and DAP10 ICD with CD3Zeta, the DAP10 is also anchored at the intracellular membrane (FIG. 7A).

Surprising, we found that both CD2 CARs and CH28H CARs designs works (CAR21, 23) well, but they don't need SAP signal (CAR22,24) (FIGS. 7B and 7C). Without being bound by any particular theory, considering CD2 and CD28H have no ITSM in the intracellular tail, reasonably, they cannot recruit SAP, therefore, SAP may not help to improve the activation signaling. Nevertheless, the CD2 CAR (CAR19) and CD28H CAR (CAR21) can overcome the HLA-I induced inhibitory signaling barrier effectively.

DNAM1 CAR is a special, we designed the DNAM1 CAR as CAR23, CAR24. DNAM1 CAR (CAR23) is effective but still limited, however, it works very well with a fourth signal SAP booster, although it does not belong to SLAMF (FIGS. 7B and 7C). Without being bound by any particular theory, DNAM1 does not belong to the SLAMF family, and has no ITSM in tail as in 2B4, SAP should not bind to it. Without being bound by any particular theory, even though DNAM1 works with LFA-1 (de Andrade et al. (2014) DNAM-1 control of natural killer cells functions through nectin and nectin-like proteins. Immunol Cell Biol. 92(3):237-44), SAP may help LFA-1 to activate Fyn, and Fyn is recruited to the tail of DNAM-1, so that enhanced the activation signal and made it completely activated.

FIGS. 7A-7E demonstrate the following results: 1. SLAMF family CD229, NTB-A, CD84, CARs are effective, and can be boosted by SAP, and CRACC CARs can be boosted by EAT2. 2. non-SLAMF CD2 and CD28H CARs are effective, and they do not need SAP for the signaling. 3. DNAM1 CAR is effective, and SAP can also boost it, although it does not belong to SLAMF.

Altogether, besides 2B4 CARs, we did a wider screening of SLAMF and non-SLAMF csCARs in NK cells, and confirmed a series of excellent CARs such as CD229 CAR (CAR12), NTB-A CAR (CAR13, CAR14), CRACC CAR (CAR16B), CD84 CAR (CAR18), CD2 CAR (CAR19), CD28H CAR (CAR 21, CAR22), and DNAM-1 CAR (CAR24), these CARs can effectively improve NK cell activation, and successfully overcome the HLA-I induced inhibitory signaling barrier, which facilitate the successful anti-tumor immunotherapy.

### 7. EQUIVALENTS

The polypeptides, polynucleotides, cells, methods, and compositions disclosed herein are not to be limited in scope by the specific embodiments described herein. Indeed, various modifications of the polypeptides, polynucleotides, cells, methods, and compositions in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

### 8. SEQUENCES

| SEQ ID | | |
|---|---|---|
| NO: | Description | Sequence |
| 1 | P2A | GSGATNFSLLKQAGDVEENPGP |
| 2 | T2A | GSGEGRGSLLRCGDVEENPGP |
| 3 | E2A | GSGQCTNYALLKLAGDVESNPGP |
| 4 | CD8 signal peptide for CAR | MALPVTALLLPLALLLHAARP |
| | expression | |
| 5 | CD8 hinge | PAKPTTTPAPRPPTPAPTIASQPLSLRPEACRPAAG |
| | | GAVHTRGLDFACD |
| 6 | CD28 hinge, TM and ICD | IHVKGKHLCPSPLFPGPSKPFWVLVVVGGVLACYSL |
| | | LVTVAFIIFWVRSKRSRLLHSDYMNMTPRRPGPTRK |
| | | HYQPYAPPRDFAAYRS |
| 7 | CD28 hinge | IHVKGKHLCPSPLFPGPSKP |
| 8 | CD28 TM | FWVLVVVGGVLACYSLLVTVAFIIFWV |
| 9 | CD28 ICD | RSKRSRLLHSDYMNMTPRRPGPTRKHYQPYAPPRDE |
| | | AAYRS |
| 10 | 4-1BB hinge, TM, and ICD | SPADLSPGASSVTPPAPAREPGHSPQIISFFLALTS |
| | | TALLFLLFELTLRFSVVKRGRKKLLYIFKQPFMRPV |
| | | QTTQEEDGCSCRFPEEEEGGCEL |
| 11 | 4-1BB hinge | SPADLSPGASSVTPPAPAREPGHSPQ |
| 12 | 4-1BB TM | IISFFLALTSTALLFLLFELTLRESVV |
| 13 | 4-1BB ICD | KRGRKKLLYIFKQPFMRPVQTTQEEDGCSCRFPEEE |
| | | EGGCEL |
| 14 | OX40 hinge, TM, and ICD | DRDPPATQPQETQGPPARPITVQPTEAWPRTSQGPS |
| | | TRPVEVPGGRAVAAILGLGLVLGLLGPLAILLALYL |
| | | LRRDQRLPPDAHKPPGGGSFRTPIQEEQADAHSTLA |
| | | KI |
| 15 | OX40 hinge | DRDPPATQPQETQGPPARPITVQPTEAWPRTSQGPS |
| | | TRPVEVPGGRA |
| 16 | OX40 TM | VAAILGLGLVLGLLGPLAILL |
| 17 | OX40 ICD | ALYLLRRDQRLPPDAHKPPGGGSFRTPIQEEQADAH |
| | | STLAKI |
| 18 | NKG2D TM | PFFFCCFIAVAMGIRFIIMVT |
| 19 | CD3zeta ICD | SFGLLDPKLCYLLDGILFIYGVILTALFLRVKFSRS |
| | | ADAPAYQQGQNQLYNELNLGRREEYDVLDKRRGRDP |
| | | EMGGKPRRKNPQEGLYNELQKDKMAEAYSEIGMKGE |
| | | RRRGKGHDGLYQGLSTATKDTYDALHMQALPPR |
| 20 | DAP10 ICD | LCARPRRSPAQEDGKVYINMPGRG |
| 21 | DAP12 ICD | YFLGRLVPRGRGAAEAATRKQRITETESPYQELQGQ |
| | | RSDVYSDLNTQRPYYK |
| 22 | 2B4 hinge, TM and ICD | SLLPDKVEKPRLQGQGKILDRGRCQVALSCLVSRDG |
| | | NVSYAWYRGSKLIQTAGNLTYLDEEVDINGTHTYTC |
| | | NVSNPVSWESHTLNLTQDCQNAHQEFREWPFLVIIV |
| | | ILSALFLGTLACFCVWRRKRKEKQSETSPKEFLTIY |
| | | EDVKDLKTRRNHEQEQTFPGGGSTIYSMIQSQSSAP |
| | | TSQEPAYTLYSLIQPSRKSGSRKRNHSPSENSTIYE |
| | | VIGKSQPKAQNPARLSRKELENFDVYS |
| 23 | 2B4 hinge | SLLPDKVEKPRLQGQGKILDRGRCQVALSCLVSRDG |
| | | NVSYAWYRGSKLIQTAGNLTYLDEEVDINGTHTYTC |
| | | NVSNPVSWESHTLNLTQDCQNAHQEFREWP |
| 24 | 2B4 TM | FLVIIVILSALFLGTLACFCV |
| 25 | 2B4 ICD | WRRKRKEKQSETSPKEFLTIYEDVKDLKTRRNHEQE |
| | | QTFPGGGSTIYSMIQSQSSAPTSQEPAYTLYSLIQP |
| | | SRKSGSRKRNHSPSFNSTIYEVIGKSQPKAQNPARL |
| | | SRKELENEDVYS |
| 26 | CD229 hinge, TM and ICD | PVSRSSHQFLSENICSGPERNTKLWIGLFLMVCLLC |
| | | VGIFSWCIWKRKGRCSVPAFCSSQAEAPADTPEPTA |
| | | GHTLYSVLSQGYEKLDTPLRPARQQPTPTSDSSSDS |
| | | NLTTEEDEDRPEVHKPISGRYEVEDQVTQEGAGHDP |
| | | APEGQADYDPVTPYVTEVESVVGENTMYAQVENLQG |
| | | KTPVSQKEESSATIYCSIRKPQVVPPPQQNDLEIPE |
| | | SPTYENFT |
| 27 | CD229 hinge | PVSRSSHQFLSENICSGPERNTK |
| 28 | CD229 TM | LWIGLFLMVCLLCVGIFSWCIW |
| 29 | CD229 ICD | KRKGRCSVPAFCSSQAEAPADTPEPTAGHTLYSVLS |
| | | QGYEKLDTPLRPARQQPTPTSDSSSDSNLTTEEDED |
| | | RPEVHKPISGRYEVFDQVTQEGAGHDPAPEGQADYD |
| | | PVTPYVTEVESVVGENTMYAQVENLQGKTPVSQKEE |
| | | SSATIYCSIRKPQVVPPPQQNDLEIPESPTYENFT |
| 30 | NTB-A hinge, TM and ICD | LRQLRNIQVTNHSQLFQNMTCELHLTCSVEDADDNV |
| | | SFRWEALGNTLSSQPNLTVSWDPRISSEQDYTCIAE |
| | | NAVSNLSFSVSAQKLCEDVKIQYTDTKMILFMVSGI |
| | | CIVFGFIILLLLVLRKRRDSLSLSTQRTQGPAESAR |
| | | NLEYVSVSPTNNTVYASVTHSNRETEIWTPRENDTI |
| | | TIYSTINHSKESKPTFSRATALDNVV |
| 31 | NTB-A hinge | LRQLRNIQVTNHSQLFQNMTCELHLTCSVEDADDNV |
| | | SFRWEALGNTLSSQPNLTVSWDPRISSEQDYTCIAE |
| | | NAVSNLSFSVSAQKLCEDVKIQYTDTKM |
| 32 | NTB-A TM | ILFMVSGICIVFGFIILLLLV |
| 33 | NTB-A ICD | LRKRRDSLSLSTQRTQGPAESARNLEYVSVSPINNT |
| | | VYASVTHSNRETEIWTPRENDTITIYSTINHSKESK |
| | | PTESRATALDNVV |
| 34 | CRACC hinge, TM and ICD | YVGIYSSSLQQPSTQEYVLHVYEHLSKPKVTMGLQS |
| | | NKNGTCVTNLTCCMEHGEEDVIYTWKALGQAANESH |
| | | NGSILPISWRWGESDMTFICVARNPVSRNESSPILA |
| | | RKLCEGAADDPDSSMVLLCLLLVPLLLSLFVLGLFL |
| | | WELKRERQEEYIEEKKRVDICRETPNICPHSGENTE |
| | | YDTIPHTNRTILKEDPANTVYSTVEIPKKMENPHSL |
| | | LTMPDTPRLFAYENVI |
| 35 | CRACC hinge | YVGIYSSSLQQPSTQEYVLHVYEHLSKPKVTMGLQS |
| | | NKNGTCVTNLTCCMEHGEEDVIYTWKALGQAANESH |
| | | NGSILPISWRWGESDMTFICVARNPVSRNESSPILA |
| | | RKLCEGAADDPDSSM |
| 36 | CRACC TM | VLLCLLLVPLLLSLFVLGLFL |
| 37 | CRACC ICD | WELKRERQEEYIEEKKRVDICRETPNICPHSGENTE |
| | | YDTIPHTNRTILKEDPANTVYSTVEIPKKMENPHSL |
| | | LTMPDTPRLFAYENVI |
| 38 | CD84 hinge, TM and ICD | PVSNNSDSISARQLCADIAMGFRTHHTGLLSVLAMF |
| | | FLLVLILSSVELERLFKRRQGRIFPEGSCLNTFTKN |
| | | PYAASKKTIYTYIMASRNTQPAESRIYDEILQSKVL |
| | | PSKEEPVNTVYSEVQFADKMGKASTQDSKPPGTSSY |
| | | EIVI |
| 39 | CD84 hinge | PVSNNSDSISARQLCADIAMGERTHHTG |
| 40 | CD84 TM | LLSVLAMFFLLVLILSSVELE |
| 41 | CD84 ICD | RLFKRRQGRIFPEGSCLNTFTKNPYAASKKTIYTYI |
| | | MASRNTQPAESRIYDEILQSKVLPSKEEPVNTVYSE |
| | | VQFADKMGKASTQDSKPPGTSSYEIVI |
| 42 | CD2 hinge, TM and ICD | IFDLKIQERVSKPKISWTCINTTLTCEVMNGTDPEL |
| | | NLYQDGKHLKLSQRVITHKWTTSLSAKFKCTAGNKV |
| | | SKESSVEPVSCPEKGLDIYLIIGICGGGSLLMVEVA |
| | | LLVFYITKRKKQRSRRNDEELETRAHRVATEERGRK |
| | | PHQIPASTPQNPATSQHPPPPPGHRSQAPSHRPPPP |
| | | GHRVQHQPQKRPPAPSGTQVHQQKGPPLPRPRVQPK |
| | | PPHGAAENSLSPSSN |
| 43 | CD2 hinge | IFDLKIQERVSKPKISWTCINTTLTCEVMNGTDPEL |
| | | NLYQDGKHLKLSQRVITHKWTTSLSAKFKCTAGNKV |
| | | SKESSVEPVSCPEKGLD |
| 44 | CD2 TM | IYLIIGICGGGSLLMVFVALLVEYIT |
| 45 | CD2 ICD | KRKKQRSRRNDEELETRAHRVATEERGRKPHQIPAS |
| | | TPQNPATSQHPPPPPGHRSQAPSHRPPPPGHRVQHQ |
| | | PQKRPPAPSGTQVHQQKGPPLPRPRVQPKPPHGAAE |
| | | NSLSPSSN |
| 46 | CD28H hinge, TM and ICD | AVEIPELEEAEGNITRLFVDPDDPTQNRNRIASFPG |
| | | FLFVLLGVGSMGVAAIVWGAWFWGRRSCQQRDSGNS |
| | | PGNAFYSNVLYRPRGAPKKSEDCSGEGKDQRGQSIY |
| | | STSFPQPAPRQPHLASRPCPSPRPCPSPRPGHPVSM |
| | | VRVSPRPSPTQQPRPKGFPKVGEE |
| 47 | CD28H hinge | AVEIPELEEAEGNITRLFVDPDDPTQNRNRIASFPG |
| 48 | CD28H TM | FLFVLLGVGSMGVAAIVWGAW |
| 49 | CD28H ICD | FWGRRSCQQRDSGNSPGNAFYSNVLYRPRGAPKKSE |
| | | DCSGEGKDQRGQSIYSTSFPQPAPRQPHLASRPCPS |
| | | PRPCPSPRPGHPVSMVRVSPRPSPTQQPRPKGFPKV |
| | | GEE |
| 50 | DNAM1 hinge, TM and ICD | AEGKTDNQYTLFVAGGTVLLLLFVISITTIIVIELN |
| | | RRRRRERRDLFTESWDTQKAPNNYRSPISTSQPTNQ |
| | | SMDDTREDIYVNYPTESRRPKTRV |
| 51 | DNAM1 hinge | AEGKTDNQYTLFVA |
| 52 | DNAM1 TM | GGTVLLLLFVISITTIIVIFL |
| 53 | DNAM1 ICD | NRRRRRERRDLFTESWDTQKAPNNYRSPISTSQPTN |
| | | QSMDDTREDIYVNYPTESRRPKTRV |
| 54 | KIR2DS1 hinge, TM, ICD | SFRDSPYEWSKSSDPLLVSVTGNPSNSWPSPTEPSS |
| | | ETGNPRHLHVLIGTSVVKIPFTILLFELLHRWCSDK |
| | | KNAAVMDQEPAGNRTVNSEDSDEQDHQEVSYA |
| 55 | KIR2DS1 hinge | SFRDSPYEWSKSSDPLLVSVTGNPSNSWPSPTEPSS |
| | | ETGNPRHLH |
| 56 | KIR2DS1 TM | VLIGTSVVKIPFTILLFELL |
| 57 | KIR2DS1 ICD | HRWCSDKKNAAVMDQEPAGNRTVNSEDSDEQDHQEV |
| | | SYA |
| 58 | KIR2DS2 hinge, TM, ICD | SFRDSPYEWSNSSDPLLVSVTGNPSNSWPSPTEPSS |
| | | KTGNPRHLHVLIGTSVVKIPFTILLFELLHRWCSNK |
| | | KNAAVMDQEPAGNRTVNSEDSDEQDHQEVSYA |
| 59 | KIR2DS2 hinge | SFRDSPYEWSNSSDPLLVSVTGNPSNSWPSPTEPSS |
| | | KTGNPRHLH |
| 60 | KIR2DS2 TM | VLIGTSVVKIPFTILLFELL |
| 61 | KIR2DS2 ICD | HRWCSNKKNAAVMDQEPAGNRTVNSEDSDEQDHQEV |
| | | SYA |
| 62 | KIR2DS3 hinge, TM, ICD | SFHDSPYEWSKSSDPLLVSVTGNPSNSWPSPTEPSS |
| | | KTGNPRHLHVLIGTSVVKLPFTILLFELLHRWCSDK |
| | | KNASVMDQGPAGNRTVNREDSDEQDHQEVSYA |
| 63 | KIR2DS3 hinge | SFHDSPYEWSKSSDPLLVSVTGNPSNSWPSPTEPSS |
| | | KTGNPRHLH |
| 64 | KIR2DS3 TM | VLIGTSVVKLPFTILLFELL |
| 65 | KIR2DS3 ICD | HRWCSDKKNASVMDQGPAGNRTVNREDSDEQDHQEV |
| | | SYA |
| 66 | KIR2DS4 hinge, TM, ICD | SFRDAPYEWSNSSDPLLVSVTGNPSNSWPSPTEPSS |
| | | KTGNPRHLHVLIGTSVVKIPFTILLFELLHRWCSDK |
| | | KNAAVMDQEPAGNRTVNSEDSDEQDHQEVSYA |
| 67 | KIR2DS4 hinge | SFRDAPYEWSNSSDPLLVSVTGNPSNSWPSPTEPSS |
| | | KTGNPRHLH |
| 68 | KIR2DS4 TM | VLIGTSVVKIPFTILLFELL |
| 69 | KIR2DS4 ICD | HRWCSDKKNAAVMDQEPAGNRTVNSEDSDEQDHQEV |
| | | SYA |
| 70 | KIR2DS5 hinge, TM, ICD | SFRDSPYEWSKSSDPLLVSVTGNSSNSWPSPTEPSS |
| | | ETGNPRHLHVLIGTSVVKLPFTILLFELLHRWCSNK |
| | | KNASVMDQGPAGNRTVNREDSDEQDHQEVSYA |
| 71 | KIR2DS5 hinge | SFRDSPYEWSKSSDPLLVSVTGNSSNSWPSPTEPSS |
| | | ETGNPRHLH |
| 72 | KIR2DS5 TM | VLIGTSVVKLPFTILLFELL |
| 73 | KIR2DS5 ICD | HRWCSNKKNASVMDQGPAGNRTVNREDSDEQDHQEV |
| | | SYA |
| 74 | KIR3DS1 hinge, TM, ICD | SFRHSPYEWSDPSDPLLVSVTGNPSSSWPSPTEPSS |
| | | KSGNLRHLHILIGTSVVKIPFTILLFELLHRWCSNK |
| | | KKCCCNGPRACREQK |
| 75 | KIR3DS1 hinge | SFRHSPYEWSDPSDPLLVSVTGNPSSSWPSPTEPSS |
| | | KSGNLRHLH |
| 76 | KIR3DS1 TM | ILIGTSVVKIPFTILLFELL |
| 77 | KIR3DS1 ICD | HRWCSNKKKCCCNGPRACREQK |
| 78 | NKG2C ICD, TM, hinge | MNKQRGTFSEVSLAQDPKRQQRKPKGNKSSISGTEQ |
| | | EIFQVELNLQNPSLNHQGIDKIYDCQGLLPPPEKLT |
| | | AEVLGIICIVLMATVLKTIVLIPFLEQNNESPNTRT |
| | | QKARHCGH |
| 79 | NKG2C ICD | MNKQRGTFSEVSLAQDPKRQQRKPKGNKSSISGTEQ |
| | | EIFQVELNLQNPSLNHQGIDKIYDCQGLLPPPEK |
| 80 | NKG2C TM | LTAEVLGIICIVLMATVLKTIVL |
| 81 | NKG2C hinge | IPFLEQNNFSPNTRTQKARHCGH |
| 82 | NKG2E ICD, TM, hinge | MSKQRGTFSEVSLAQDPKWQQRKPKGNKSSISGTEQ |
| | | EIFQVELNLQNASLNHQGIDKIYDCQGLLPPPEKLT |
| | | AEVLGIICIVLMATVLKTIVLIPFLEQNNSSPNART |
| | | QKARHCGH |
| 83 | NKG2E ICD | MSKQRGTFSEVSLAQDPKWQQRKPKGNKSSISGTEQ |
| | | EIFQVELNLQNASLNHQGIDKIYDCQGLLPPPEK |
| 84 | NKG2E TM | LTAEVLGIICIVLMATVLKTIVL |
| 85 | NKG2E hinge | IPFLEQNNSSPNARTQKARHCGH |
| 86 | SAP | MDAVAVYHGKISRETGEKLLLATGLDGSYLLRDSES |
| | | VPGVYCLCVLYHGYIYTYRVSQTETGSWSAETAPGV |
| | | HKRYFRKIKNLISAFQKPDQGIVIPLQYPVEKKSSA |
| | | RSTQGTTGIREDPDVCLKAP |
| 87 | EAT2 | MDLPYYHGRLTKQDCETLLLKEGVDGNELLRDSESI |
| | | PGVLCLCVSFKNIVYTYRIFREKHGYYRIQTAEGSP |
| | | KQVFPSLKELISKFEKPNQGMVVHLLKPIKRTSPSL |
| | | RWRGLKLELETFVNSNSDYVDVLP |
| 88 | MICA | MGLGPVELLLAGIFPFAPPGAAAEPHSLRYNLTVLS |
| | | WDGSVQSGFLTEVHLDGQPFLRCDRQKCRAKPQGQW |
| | | AEDVLGNKTWDRETRDLTGNGKDLRMTLAHIKDQKE |
| | | GLHSLQEIRVCEIHEDNSTRSSQHFYYDGELFLSQN |
| | | LETKEWTMPQSSRAQTLAMNVRNFLKEDAMKTKTHY |
| | | HAMHADCLQELRRYLKSGVVLRRTVPPMVNVTRSEA |
| | | SEGNITVTCRASGFYPWNITLSWRQDGVSLSHDTQQ |
| | | WGDVLPDGNGTYQTWVATRICQGEEQRFTCYMEHSG |
| | | NHSTHPVPSGKVLVLQSHWQTFHVSAVAAAAIFVII |
| | | IFYVRCCKKKTSAAEGPELVSLQVLDQHPVGTSDHR |
| | | DATQLGFQPLMSDLGSTGSTEGA |
| 89 | MICB | MGLGRVLLFLAVAFPFAPPAAAAEPHSLRYNLMVLS |
| | | QDGSVQSGFLAEGHLDGQPFLRYDRQKRRAKPQGQW |
| | | AENVLGAKTWDTETEDLTENGQDLRRTLTHIKDQKG |
| | | GLHSLQEIRVCEIHEDSSTRGSRHFYYDGELFLSQN |
| | | LETQESTVPQSSRAQTLAMNVTNEWKEDAMKTKTHY |
| | | RAMQADCLQKLQRYLKSGVAIRRTVPPMVNVTCSEV |
| | | SEGNITVTCRASSFYPRNITLTWRQDGVSLSHNTQQ |
| | | WGDVLPDGNGTYQTWVATRIRQGEEQRFTCYMEHSG |
| | | NHGTHPVPSGKALVLQSQRTDFPYVSAAMPCFVIII |
| | | ILCVPCCKKKTSAAEGPELVSLQVLDQHPVGTGDHR |
| | | DAAQLGFQPLMSATGSTGSTEGT |
| 90 | HLA-E | MVDGTLLLLLSEALALTQTWAGSHSLKYFHTSVSRP |
| | | GRGEPRFISVGYVDDTQFVREDNDAASPRMVPRAPW |
| | | MEQEGSEYWDRETRSARDTAQIFRVNLRTLRGYYNQ |
| | | SEAGSHTLQWMHGCELGPDGRFLRGYEQFAYDGKDY |
| | | LTLNEDLRSWTAVDTAAQISEQKSNDASEAEHQRAY |
| | | LEDTCVEWLHKYLEKGKETLLHLEPPKTHVTHHPIS |
| | | DHEATLRCWALGFYPAEITLTWQQDGEGHTQDTELV |
| | | ETRPAGDGTFQKWAAVVVPSGEEQRYTCHVQHEGLP |
| | | EPVTLRWKPASQPTIPIVGIIAGLVLLGSVVSGAVV |
| | | AAVIWRKKSSGGKGGSYSKAEWSDSAQGSESHSL |
| 91 | Membrane bound CD48 | MALPVTALLLPLALLLHAARPHLVHMTVVSGSNVTL |
| | | NISESLPENYKQLTWFYTFDQKIVEWDSRKSKYFES |
| | | KFKGRVRLDPQSGALYISKVQKEDNSTYIMRVLKKT |
| | | GNEQEWKIKLQVLDPVPKPVIKIEKIEDMDDNCYLK |
| | | LSCVIPGESVNYTWYGDKRPFPKELQNSVLETTLMP |
| | | HNYSRCYTCQVSNSVSSKNGTVCLSPPCTLGKKDPW |
| | | ELRGAQGNWSCFEQRKAGGPIQPPCTVWWIYIWAPL |
| | | AGTCGVLLLSLVIT |
| 92 | GPC3 | MAGTVRTACLVVAMLLSLDFPGQAQPPPPPPDATCH |
| | | QVRSFFQRLQPGLKWVPETPVPGSDLQVCLPKGPTC |
| | | CSRKMEEKYQLTARLNMEQLLQSASMELKFLIIQNA |
| | | AVFQEAFEIVVRHAKNYTNAMFKNNYPSLTPQAFEF |
| | | VGEFFTDVSLYILGSDINVDDMVNELFDSLFPVIYT |
| | | QLMNPGLPDSALDINECLRGARRDLKVEGNFPKLIM |
| | | TQVSKSLQVTRIFLQALNLGIEVINTTDHLKESKDC |
| | | GRMLTRMWYCSYCQGLMMVKPCGGYCNVVMQGCMAG |
| | | VVEIDKYWREYILSLEELVNGMYRIYDMENVLLGLE |
| | | STIHDSIQYVQKNAGKLTTTETEKKIWHFKYPIFFL |
| | | CIGLDLQIGKLCAHSQQRQYRSAYYPEDLFIDKKVL |
| | | KVAHVEHEETLSSRRRELIQKLKSFISFYSALPGYI |
| | | CSHSPVAENDTLCWNGQELVERYSQKAARNGMKNQF |
| | | NLHELKMKGPEPVVSQIIDKLKHINQLLRTMSMPKG |
| | | RVLDKNLDEEGFESGDCGDDEDECIGGSGDGMIKVK |
| | | NQLRFLAELAYDLDVDDAPGNSQQATPKDNEISTFH |
| | | NLGNVHSPLKLLTSMAISVVCFFELVH |
| 93 | CD19 | MPPPRLLFFLLELTPMEVRPEEPLVVKVEEGDNAVL |
| | | QCLKGTSDGPTQQLTWSRESPLKPFLKLSLGLPGLG |
| | | IHMRPLAIWLFIENVSQQMGGFYLCQPGPPSEKAWQ |
| | | PGWTVNVEGSGELFRWNVSDLGGLGCGLKNRSSEGP |
| | | SSPSGKLMSPKLYVWAKDRPEIWEGEPPCLPPRDSL |
| | | NQSLSQDLTMAPGSTLWLSCGVPPDSVSRGPLSWTH |
| | | VHPKGPKSLLSLELKDDRPARDMWVMETGLLLPRAT |
| | | AQDAGKYYCHRGNLTMSFHLEITARPVLWHWLLRTG |
| | | GWKVSAVTLAYLIFCLCSLVGILHL |
| 94 | CD137L | MEYASDASLDPEAPWPPAPRARACRVLPWALVAGLL |
| | | LLLLLAAACAVELACPWAVSGARASPGSAASPRLRE |
| | | GPELSPDDPAGLLDLRQGMFAQLVAQNVLLIDGPLS |
| | | WYSDPGLAGVSLTGGLSYKEDTKELVVAKAGVYYVF |
| | | FQLELRRVVAGEGSGSVSLALHLQPLRSAAGAAALA |
| | | LTVDLPPASSEARNSAFGFQGRLLHLSAGQRLGVHL |
| | | HTEARARHAWQLTQGATVLGLFRVTPEIPAGLPSPR |
| | | SE |
| 95 | LUC2 | MEDAKNIKKGPAPFYPLEDGTAGEQLHKAMKRYALV |
| | | PGTIAFTDAHIEVDITYAEYFEMSVRLAEAMKRYGL |
| | | NTNHRIVVCSENSLQFFMPVLGALFIGVAVAPANDI |
| | | YNERELLNSMGISQPTVVFVSKKGLQKILNVQKKLP |
| | | IIQKIIIMDSKTDYQGFQSMYTFVTSHLPPGENEYD |
| | | FVPESEDRDKTIALIMNSSGSTGLPKGVALPHRTAC |
| | | VRFSHARDPIFGNQIIPDTAILSVVPFHHGFGMFTT |
| | | LGYLICGFRVVLMYRFEEELFLRSLQDYKIQSALLV |
| | | PTLESFFAKSTLIDKYDLSNLHEIASGGAPLSKEVG |
| | | EAVAKRFHLPGIRQGYGLTETTSAILITPEGDDKPG |
| | | AVGKVVPFFEAKVVDLDTGKTLGVNQRGELCVRGPM |
| | | IMSGYVNNPEATNALIDKDGWLHSGDIAYWDEDEHE |
| | | FIVDRLKSLIKYKGYQVAPAELESILLQHPNIFDAG |
| | | VAGLPDDDAGELPAAVVVLEHGKTMTEKEIVDYVAS |
| | | QVTTAKKLRGGVVFVDEVPKGLTGKLDARKIREILI |
| | | KAKKGGKIAV |
| 96 | CD28 hinge/TM/ICD- | IEVMYPPPYLDNEKSNGTIIHVKGKHLCPSPLFPGP |
| | CD3zeta in CAR1 in T CAR | SKPFWVLVVVGGVLACYSLLVTVAFIIFWVRSKRSR |
| | | LLHSDYMNMTPRRPGPTRKHYQPYAPPRDFAAYRSR |
| | | VKFSRSADAPAYQQGQNQLYNELNLGRREEYDVLDK |
| | | RRGRDPEMGGKPRRKNPQEGLYNELQKDKMAEAYSE |
| | | IGMKGERRRGKGHDGLYQGLSTATKDTYDALHMQAL |
| | | PPR |
| 97 | 2B4 hinge/TM/ICD in CAR1 | SLLPDKVEKPRLQGQGKILDRGRCQVALSCLVSRDG |
| | | NVSYAWYRGSKLIQTAGNLTYLDEEVDINGTHTYTC |
| | | NVSNPVSWESHTLNLTQDCQNAHQEFREWPFLVIIV |
| | | ILSALFLGTLACFCVWRRKRKEKQSETSPKEFLTIY |
| | | EDVKDLKTRRNHEQEQTFPGGGSTIYSMIQSQSSAP |
| | | TSQEPAYTLYSLIQPSRKSGSRKRNHSPSENSTIYE |
| | | VIGKSQPKAQNPARLSRKELENEDVYS |
| 98 | CD8 hinge-NKG2DTM- | PAKPTTTPAPRPPTPAPTIASQPLSLRPEACRPAAG |
| | 2B4ICD-CD3zeta in CAR2 | GAVHTRGLDFACDPFFFCCFIAVAMGIRFIIMVTWR |
| | | RKRKEKQSETSPKEFLTIYEDVKDLKTRRNHEQEQT |
| | | FPGGGSTIYSMIQSQSSAPTSQEPAYTLYSLIQPSR |
| | | KSGSRKRNHSPSFNSTIYEVIGKSQPKAQNPARLSR |
| | | KELENFDVYSSFGLLDPKLCYLLDGILFIYGVILTA |
| | | LFLRVKFSRSADAPAYQQGQNQLYNELNLGRREEYD |
| | | VLDKRRGRDPEMGGKPRRKNPQEGLYNELQKDKMAE |
| | | AYSEIGMKGERRRGKGHDGLYQGLSTATKDTYDALH |
| | | MQALPPR |
| 99 | CD8 hinge-NKG2DTM- | PAKPTTTPAPRPPTPAPTIASQPLSLRPEACRPAAG |
| | 2B4ICD-CD3zeta-P2A-SAP | GAVHTRGLDFACDPFFFCCFIAVAMGIRFIIMVTWR |
| | in CAR3 | RKRKEKQSETSPKEFLTIYEDVKDLKTRRNHEQEQT |
| | | FPGGGSTIYSMIQSQSSAPTSQEPAYTLYSLIQPSR |
| | | KSGSRKRNHSPSFNSTIYEVIGKSQPKAQNPARLSR |
| | | KELENFDVYSSFGLLDPKLCYLLDGILFIYGVILTA |
| | | LFLRVKESRSADAPAYQQGQNQLYNELNLGRREEYD |
| | | VLDKRRGRDPEMGGKPRRKNPQEGLYNELQKDKMAE |
| | | AYSEIGMKGERRRGKGHDGLYQGLSTATKDTYDALH |
| | | MQALPPRGSGATNFSLLKQAGDVEENPGPMDAVAVY |
| | | HGKISRETGEKLLLATGLDGSYLLRDSESVPGVYCL |
| | | CVLYHGYIYTYRVSQTETGSWSAETAPGVHKRYFRK |
| | | IKNLISAFQKPDQGIVIPLQYPVEKKSSARSTQGTT |
| | | GIREDPDVCLKAP |
| 100 | ScFv-CD8 hinge- | PAKPTTTPAPRPPTPAPTIASQPLSLRPEACRPAAG |
| | 2B4TM/ICD-DAP10- | GAVHTRGLDFACDELVIIVILSALFLGTLACFCVWR |
| | CD3zeta in CAR4 | RKRKEKQSETSPKEFLTIYEDVKDLKTRRNHEQEQT |
| | | FPGGGSTIYSMIQSQSSAPTSQEPAYTLYSLIQPSR |
| | | KSGSRKRNHSPSENSTIYEVIGKSQPKAQNPARLSR |
| | | KELENFDVYSLCARPRRSPAQEDGKVYINMPGRGSF |
| | | GLLDPKLCYLLDGILFIYGVILTALFLRVKESRSAD |
| | | APAYQQGQNQLYNELNLGRREEYDVLDKRRGRDPEM |
| | | GGKPRRKNPQEGLYNELQKDKMAEAYSEIGMKGERR |
| | | RGKGHDGLYQGLSTATKDTYDALHMQALPPR |
| 101 | CD8 hinge-2B4TM/ICD- | PAKPTTTPAPRPPTPAPTIASQPLSLRPEACRPAAG |
| | CD3zeta in CAR4A | GAVHTRGLDFACDELVIIVILSALFLGTLACFCVWR |
| | | RKRKEKQSETSPKEFLTIYEDVKDLKTRRNHEQEQT |
| | | FPGGGSTIYSMIQSQSSAPTSQEPAYTLYSLIQPSR |
| | | KSGSRKRNHSPSFNSTIYEVIGKSQPKAQNPARLSR |
| | | KELENFDVYSSFGLLDPKLCYLLDGILFIYGVILTA |
| | | LFLRVKFSRSADAPAYQQGQNQLYNELNLGRREEYD |
| | | VLDKRRGRDPEMGGKPRRKNPQEGLYNELQKDKMAE |
| | | AYSEIGMKGERRRGKGHDGLYQGLSTATKDTYDALH |
| | | MQALPPR |
| 102 | CD8 hinge-2B4TM-DAP10- | PAKPTTTPAPRPPTPAPTIASQPLSLRPEACRPAAG |
| | 2B4ICD-CD3zeta in CAR4B | GAVHTRGLDFACDFWVLVVVGGVLACYSLLVTVAFI |
| | | IFWVLCARPRRSPAQEDGKVYINMPGRGWRRKRKEK |
| | | QSETSPKEFLTIYEDVKDLKTRRNHEQEQTFPGGGS |
| | | TIYSMIQSQSSAPTSQEPAYTLYSLIQPSRKSGSRK |
| | | RNHSPSENSTIYEVIGKSQPKAQNPARLSRKELENF |
| | | DVYSSFGLLDPKLCYLLDGILFIYGVILTALFLRVK |
| | | FSRSADAPAYQQGQNQLYNELNLGRREEYDVLDKRR |
| | | GRDPEMGGKPRRKNPQEGLYNELQKDKMAEAYSEIG |
| | | MKGERRRGKGHDGLYQGLSTATKDTYDALHMQALPP |
| | | R |
| 103 | CD8 hinge-2B4TM/ICD- | PAKPTTTPAPRPPTPAPTIASQPLSLRPEACRPAAG |
| | DAP10 in CAR4C | GAVHTRGLDFACDELVIIVILSALFLGTLACFCVWR |
| | | RKRKEKQSETSPKEFLTIYEDVKDLKTRRNHEQEQT |
| | | FPGGGSTIYSMIQSQSSAPTSQEPAYTLYSLIQPSR |
| | | KSGSRKRNHSPSFNSTIYEVIGKSQPKAQNPARLSR |
| | | KELENFDVYSLCARPRRSPAQEDGKVYINMPGRG |
| 104 | CD8 hinge-2B4TM/ICD- | PAKPTTTPAPRPPTPAPTIASQPLSLRPEACRPAAG |
| | DAP10-CD3zeta-P2A-SAP | GAVHTRGLDFACDELVIIVILSALFLGTLACFCVWR |
| | in CAR5 | RKRKEKQSETSPKEFLTIYEDVKDLKTRRNHEQEQT |
| | | FPGGGSTIYSMIQSQSSAPTSQEPAYTLYSLIQPSR |
| | | KSGSRKRNHSPSENSTIYEVIGKSQPKAQNPARLSR |
| | | KELENFDVYSLCARPRRSPAQEDGKVYINMPGRGSF |
| | | GLLDPKLCYLLDGILFIYGVILTALFLRVKESRSAD |
| | | APAYQQGQNQLYNELNLGRREEYDVLDKRRGRDPEM |
| | | GGKPRRKNPQEGLYNELQKDKMAEAYSEIGMKGERR |
| | | RGKGHDGLYQGLSTATKDTYDALHMQALPPRGSGAT |
| | | NFSLLKQAGDVEENPGPMDAVAVYHGKISRETGEKL |
| | | LLATGLDGSYLLRDSESVPGVYCLCVLYHGYIYTYR |
| | | VSQTETGSWSAETAPGVHKRYFRKIKNLISAFQKPD |
| | | QGIVIPLQYPVEKKSSARSTQGTTGIREDPDVCLKA |
| | | P |
| 105 | CD8 hinge-2B4TM-DAP10- | PAKPTTTPAPRPPTPAPTIASQPLSLRPEACRPAAG |
| | 2B4ICD-CD3zeta-P2A-SAP | GAVHTRGLDFACDELVIIVILSALFLGTLACFCVLC |
| | in CAR5B | ARPRRSPAQEDGKVYINMPGRGWRRKRKEKQSETSP |
| | | KEFLTIYEDVKDLKTRRNHEQEQTFPGGGSTIYSMI |
| | | QSQSSAPTSQEPAYTLYSLIQPSRKSGSRKRNHSPS |
| | | FNSTIYEVIGKSQPKAQNPARLSRKELENEDVYSSF |
| | | GLLDPKLCYLLDGILFIYGVILTALFLRVKESRSAD |
| | | APAYQQGQNQLYNELNLGRREEYDVLDKRRGRDPEM |
| | | GGKPRRKNPQEGLYNELQKDKMAEAYSEIGMKGERR |
| | | RGKGHDGLYQGLSTATKDTYDALHMQALPPRGSGAT |
| | | NFSLLKQAGDVEENPGPMDAVAVYHGKISRETGEKL |
| | | LLATGLDGSYLLRDSESVPGVYCLCVLYHGYIYTYR |
| | | VSQTETGSWSAETAPGVHKRYFRKIKNLISAFQKPD |
| | | QGIVIPLQYPVEKKSSARSTQGTTGIREDPDVCLKA |
| | | P |
| 106 | 2B4 hinge/TM/ICD-DAP10- | SLLPDKVEKPRLQGQGKILDRGRCQVALSCLVSRDG |
| | CD3zeta in CAR6 | NVSYAWYRGSKLIQTAGNLTYLDEEVDINGTHTYTC |
| | | NVSNPVSWESHTLNLTQDCQNAHQEFREWPELVIIV |
| | | ILSALFLGTLACFCVWRRKRKEKQSETSPKEFLTIY |
| | | EDVKDLKTRRNHEQEQTFPGGGSTIYSMIQSQSSAP |
| | | TSQEPAYTLYSLIQPSRKSGSRKRNHSPSENSTIYE |
| | | VIGKSQPKAQNPARLSRKELENFDVYSLCARPRRSP |
| | | AQEDGKVYINMPGRGSFGLLDPKLCYLLDGILFIYG |
| | | VILTALFLRVKESRSADAPAYQQGQNQLYNELNLGR |
| | | REEYDVLDKRRGRDPEMGGKPRRKNPQEGLYNELQK |
| | | DKMAEAYSEIGMKGERRRGKGHDGLYQGLSTATKDT |
| | | YDALHMQALPPR |
| 107 | 2B4 hinge/TM-DAP10- | SLLPDKVEKPRLQGQGKILDRGRCQVALSCLVSRDG |
| | 2B4ICD-CD3zeta in CAR6B | NVSYAWYRGSKLIQTAGNLTYLDEEVDINGTHTYTC |
| | | NVSNPVSWESHTLNLTQDCQNAHQEFREWPELVIIV |
| | | ILSALFLGTLACFCVLCARPRRSPAQEDGKVYINMP |
| | | GRGWRRKRKEKQSETSPKEFLTIYEDVKDLKTRRNH |
| | | EQEQTFPGGGSTIYSMIQSQSSAPTSQEPAYTLYSL |
| | | IQPSRKSGSRKRNHSPSENSTIYEVIGKSQPKAQNP |
| | | ARLSRKELENEDVYSSFGLLDPKLCYLLDGILFIYG |
| | | VILTALFLRVKFSRSADAPAYQQGQNQLYNELNLGR |
| | | REEYDVLDKRRGRDPEMGGKPRRKNPQEGLYNELQK |
| | | DKMAEAYSEIGMKGERRRGKGHDGLYQGLSTATKDT |
| | | YDALHMQALPPR |
| 108 | 2B4 hinge/TM/ICD-DAP10- | SLLPDKVEKPRLQGQGKILDRGRCQVALSCLVSRDG |
| | CD3zeta-P2A-SAP in CAR8 | NVSYAWYRGSKLIQTAGNLTYLDEEVDINGTHTYTC |
| | | NVSNPVSWESHTLNLTQDCQNAHQEFREWPFLVIIV |
| | | ILSALFLGTLACFCVWRRKRKEKQSETSPKEFLTIY |
| | | EDVKDLKTRRNHEQEQTFPGGGSTIYSMIQSQSSAP |
| | | TSQEPAYTLYSLIQPSRKSGSRKRNHSPSENSTIYE |
| | | VIGKSQPKAQNPARLSRKELENFDVYSLCARPRRSP |
| | | AQEDGKVYINMPGRGSFGLLDPKLCYLLDGILFIYG |
| | | VILTALFLRVKESRSADAPAYQQGQNQLYNELNLGR |
| | | REEYDVLDKRRGRDPEMGGKPRRKNPQEGLYNELQK |
| | | DKMAEAYSEIGMKGERRRGKGHDGLYQGLSTATKDT |
| | | YDALHMQALPPRGSGATNFSLLKQAGDVEENPGPMD |
| | | AVAVYHGKISRETGEKLLLATGLDGSYLLRDSESVP |
| | | GVYCLCVLYHGYIYTYRVSQTETGSWSAETAPGVHK |
| | | RYFRKIKNLISAFQKPDQGIVIPLQYPVEKKSSARS |
| | | TQGTTGIREDPDVCLKAP |
| 109 | 2B4 hinge/TM-DAP10- | SLLPDKVEKPRLQGQGKILDRGRCQVALSCLVSRDG |
| | 2B4ICD-CD3zeta-P2A-SAP | NVSYAWYRGSKLIQTAGNLTYLDEEVDINGTHTYTC |
| | in CAR8B | NVSNPVSWESHTLNLTQDCQNAHQEFREWPELVIIV |
| | | ILSALFLGTLACFCVLCARPRRSPAQEDGKVYINMP |
| | | GRGWRRKRKEKQSETSPKEFLTIYEDVKDLKTRRNH |
| | | EQEQTFPGGGSTIYSMIQSQSSAPTSQEPAYTLYSL |
| | | IQPSRKSGSRKRNHSPSENSTIYEVIGKSQPKAQNP |
| | | ARLSRKELENFDVYSSFGLLDPKLCYLLDGILFIYG |
| | | VILTALFLRVKFSRSADAPAYQQGQNQLYNELNLGR |
| | | REEYDVLDKRRGRDPEMGGKPRRKNPQEGLYNELQK |
| | | DKMAEAYSEIGMKGERRRGKGHDGLYQGLSTATKDT |
| | | YDALHMQALPPRGSGATNESLLKQAGDVEENPGPMD |
| | | AVAVYHGKISRETGEKLLLATGLDGSYLLRDSESVP |
| | | GVYCLCVLYHGYIYTYRVSQTETGSWSAETAPGVHK |
| | | RYFRKIKNLISAFQKPDQGIVIPLQYPVEKKSSARS |
| | | TQGTTGIREDPDVCLKAP |
| 110 | 2B4 hinge/TM/ICD-DAP10- | SLLPDKVEKPRLQGQGKILDRGRCQVALSCLVSRDG |
| | CD3zeta-P2A-EAT2 in | NVSYAWYRGSKLIQTAGNLTYLDEEVDINGTHTYTC |
| | CAR10 | NVSNPVSWESHTLNLTQDCQNAHQEFREWPFLVIIV |
| | | ILSALFLGTLACFCVWRRKRKEKQSETSPKEFLTIY |
| | | EDVKDLKTRRNHEQEQTFPGGGSTIYSMIQSQSSAP |
| | | TSQEPAYTLYSLIQPSRKSGSRKRNHSPSENSTIYE |
| | | VIGKSQPKAQNPARLSRKELENFDVYSLCARPRRSP |
| | | AQEDGKVYINMPGRGSFGLLDPKLCYLLDGILFIYG |
| | | VILTALFLRVKFSRSADAPAYQQGQNQLYNELNLGR |
| | | REEYDVLDKRRGRDPEMGGKPRRKNPQEGLYNELQK |
| | | DKMAEAYSEIGMKGERRRGKGHDGLYQGLSTATKDT |
| | | YDALHMQALPPRGSGATNESLLKQAGDVEENPGPMD |
| | | LPYYHGRLTKQDCETLLLKEGVDGNELLRDSESIPG |
| | | VLCLCVSFKNIVYTYRIFREKHGYYRIQTAEGSPKQ |
| | | VFPSLKELISKFEKPNQGMVVHLLKPIKRTSPSLRW |
| | | RGLKLELETFVNSNSDYVDVLP |
| 111 | CD229 hinge/TM-DAP10- | PVSRSSHQFLSENICSGPERNTKLWIGLFLMVCLLC |
| | CD229ICD-CD3zeta in | VGIFSWCIWLCARPRRSPAQEDGKVYINMPGRGKRK |
| | CAR11 | GRCSVPAFCSSQAEAPADTPEPTAGHTLYSVLSQGY |
| | | EKLDTPLRPARQQPTPTSDSSSDSNLTTEEDEDRPE |
| | | VHKPISGRYEVFDQVTQEGAGHDPAPEGQADYDPVT |
| | | PYVTEVESVVGENTMYAQVENLQGKTPVSQKEESSA |
| | | TIYCSIRKPQVVPPPQQNDLEIPESPTYENFTSFGL |
| | | LDPKLCYLLDGILFIYGVILTALFLRVKFSRSADAP |
| | | AYQQGQNQLYNELNLGRREEYDVLDKRRGRDPEMGG |
| | | KPRRKNPQEGLYNELQKDKMAEAYSEIGMKGERRRG |
| | | KGHDGLYQGLSTATKDTYDALHMQALPPR |
| 112 | CD229 hinge/TM-DAP10- | PVSRSSHQFLSENICSGPERNTKLWIGLFLMVCLLC |
| | CD229ICD-CD3zeta-P2A- | VGIFSWCIWLCARPRRSPAQEDGKVYINMPGRGKRK |
| | SAP in CAR12 | GRCSVPAFCSSQAEAPADTPEPTAGHTLYSVLSQGY |
| | | EKLDTPLRPARQQPTPTSDSSSDSNLTTEEDEDRPE |
| | | VHKPISGRYEVEDQVTQEGAGHDPAPEGQADYDPVT |
| | | PYVTEVESVVGENTMYAQVENLQGKTPVSQKEESSA |
| | | TIYCSIRKPQVVPPPQQNDLEIPESPTYENFTSFGL |
| | | LDPKLCYLLDGILFIYGVILTALFLRVKFSRSADAP |
| | | AYQQGQNQLYNELNLGRREEYDVLDKRRGRDPEMGG |
| | | KPRRKNPQEGLYNELQKDKMAEAYSEIGMKGERRRG |
| | | KGHDGLYQGLSTATKDTYDALHMQALPPRGSGATNE |
| | | SLLKQAGDVEENPGPMDAVAVYHGKISRETGEKLLL |
| | | ATGLDGSYLLRDSESVPGVYCLCVLYHGYIYTYRVS |
| | | QTETGSWSAETAPGVHKRYFRKIKNLISAFQKPDQG |
| | | IVIPLQYPVEKKSSARSTQGTTGIREDPDVCLKAP |
| 113 | NTB-A hinge/TM-DAP10- | LRQLRNIQVTNHSQLFQNMTCELHLTCSVEDADDNV |
| | NTB-AICD-CD3zeta in | SFRWEALGNTLSSQPNLTVSWDPRISSEQDYTCIAE |
| | CAR13 | NAVSNLSFSVSAQKLCEDVKIQYTDTKMILEMVSGI |
| | | CIVFGFIILLLLVLCARPRRSPAQEDGKVYINMPGR |
| | | GLRKRRDSLSLSTQRTQGPAESARNLEYVSVSPTNN |
| | | TVYASVTHSNRETEIWTPRENDTITIYSTINHSKES |
| | | KPTFSRATALDNVVSFGLLDPKLCYLLDGILFIYGV |
| | | ILTALFLRVKESRSADAPAYQQGQNQLYNELNLGRR |
| | | EEYDVLDKRRGRDPEMGGKPRRKNPQEGLYNELQKD |
| | | KMAEAYSEIGMKGERRRGKGHDGLYQGLSTATKDTY |
| | | DALHMQALPPR |
| 114 | NTB-A hinge/TM-DAP10- | LRQLRNIQVTNHSQLFQNMTCELHLTCSVEDADDNV |
| | NTB-AICD-CD3zeta-P2A- | SFRWEALGNTLSSQPNLTVSWDPRISSEQDYTCIAE |
| | SAP in CAR14 | NAVSNLSFSVSAQKLCEDVKIQYTDTKMILFMVSGI |
| | | CIVFGFIILLLLVLCARPRRSPAQEDGKVYINMPGR |
| | | GLRKRRDSLSLSTQRTQGPAESARNLEYVSVSPTNN |
| | | TVYASVTHSNRETEIWTPRENDTITIYSTINHSKES |
| | | KPTFSRATALDNVVSFGLLDPKLCYLLDGILFIYGV |
| | | ILTALFLRVKFSRSADAPAYQQGQNQLYNELNLGRR |
| | | EEYDVLDKRRGRDPEMGGKPRRKNPQEGLYNELQKD |
| | | KMAEAYSEIGMKGERRRGKGHDGLYQGLSTATKDTY |
| | | DALHMQALPPRGSGATNFSLLKQAGDVEENPGPMDA |
| | | VAVYHGKISRETGEKLLLATGLDGSYLLRDSESVPG |
| | | VYCLCVLYHGYIYTYRVSQTETGSWSAETAPGVHKR |
| | | YFRKIKNLISAFQKPDQGIVIPLQYPVEKKSSARST |
| | | QGTTGIREDPDVCLKAP |
| 115 | CRACC hinge/TM-DAP10- | YVGIYSSSLQQPSTQEYVLHVYEHLSKPKVTMGLQS |
| | CRACCICD-CD3zeta in | NKNGTCVTNLTCCMEHGEEDVIYTWKALGQAANESH |
| | CAR15 | NGSILPISWRWGESDMTFICVARNPVSRNESSPILA |
| | | RKLCEGAADDPDSSMVLLCLLLVPLLLSLFVLGLEL |
| | | LCARPRRSPAQEDGKVYINMPGRGWELKRERQEEYI |
| | | EEKKRVDICRETPNICPHSGENTEYDTIPHTNRTIL |
| | | KEDPANTVYSTVEIPKKMENPHSLLTMPDTPRLFAY |
| | | ENVISFGLLDPKLCYLLDGILFIYGVILTALFLRVK |
| | | FSRSADAPAYQQGQNQLYNELNLGRREEYDVLDKRR |
| | | GRDPEMGGKPRRKNPQEGLYNELQKDKMAEAYSEIG |
| | | MKGERRRGKGHDGLYQGLSTATKDTYDALHMQALPP |
| | | R |
| 116 | CRACC hinge/TM-DAP10- | YVGIYSSSLQQPSTQEYVLHVYEHLSKPKVTMGLQS |
| | CRACCICD-CD3zeta-P2A- | NKNGTCVTNLTCCMEHGEEDVIYTWKALGQAANESH |
| | SAP in CAR16 | NGSILPISWRWGESDMTFICVARNPVSRNESSPILA |
| | | RKLCEGAADDPDSSMVLLCLLLVPLLLSLFVLGLFL |
| | | LCARPRRSPAQEDGKVYINMPGRGWELKRERQEEYI |
| | | EEKKRVDICRETPNICPHSGENTEYDTIPHTNRTIL |
| | | KEDPANTVYSTVEIPKKMENPHSLLTMPDTPRLFAY |
| | | ENVISFGLLDPKLCYLLDGILFIYGVILTALFLRVK |
| | | FSRSADAPAYQQGQNQLYNELNLGRREEYDVLDKRR |
| | | GRDPEMGGKPRRKNPQEGLYNELQKDKMAEAYSEIG |
| | | MKGERRRGKGHDGLYQGLSTATKDTYDALHMQALPP |
| | | RGSGATNFSLLKQAGDVEENPGPMDAVAVYHGKISR |
| | | ETGEKLLLATGLDGSYLLRDSESVPGVYCLCVLYHG |
| | | YIYTYRVSQTETGSWSAETAPGVHKRYFRKIKNLIS |
| | | AFQKPDQGIVIPLQYPVEKKSSARSTQGTTGIREDP |
| | | DVCLKAP |
| 117 | CRACC hinge/TM-DAP10- | YVGIYSSSLQQPSTQEYVLHVYEHLSKPKVTMGLQS |
| | CRACCICD-CD3zeta-P2A- | NKNGTCVTNLTCCMEHGEEDVIYTWKALGQAANESH |
| | EAT2 in CAR16B | NGSILPISWRWGESDMTFICVARNPVSRNESSPILA |
| | | RKLCEGAADDPDSSMVLLCLLLVPLLLSLFVLGLEL |
| | | LCARPRRSPAQEDGKVYINMPGRGWELKRERQEEYI |
| | | EEKKRVDICRETPNICPHSGENTEYDTIPHTNRTIL |
| | | KEDPANTVYSTVEIPKKMENPHSLLTMPDTPRLFAY |
| | | ENVISFGLLDPKLCYLLDGILFIYGVILTALFLRVK |
| | | FSRSADAPAYQQGQNQLYNELNLGRREEYDVLDKRR |
| | | GRDPEMGGKPRRKNPQEGLYNELQKDKMAEAYSEIG |
| | | MKGERRRGKGHDGLYQGLSTATKDTYDALHMQALPP |
| | | RGSGATNFSLLKQAGDVEENPGPMDLPYYHGRLTKQ |
| | | DCETLLLKEGVDGNFLLRDSESIPGVLCLCVSEKNI |
| | | VYTYRIFREKHGYYRIQTAEGSPKQVFPSLKELISK |
| | | FEKPNQGMVVHLLKPIKRTSPSLRWRGLKLELETFV |
| | | NSNSDYVDVLP |
| 118 | CD84 hinge/TM-DAP10- | PVSNNSDSISARQLCADIAMGERTHHTGLLSVLAME |
| | CD84ICD-CD3zeta in | FLLVLILSSVELFLCARPRRSPAQEDGKVYINMPGR |
| | CAR17 | GRLFKRRQGRIFPEGSCLNTFTKNPYAASKKTIYTY |
| | | IMASRNTQPAESRIYDEILQSKVLPSKEEPVNTVYS |
| | | EVQFADKMGKASTQDSKPPGTSSYEIVISFGLLDPK |
| | | LCYLLDGILFIYGVILTALFLRVKESRSADAPAYQQ |
| | | GQNQLYNELNLGRREEYDVLDKRRGRDPEMGGKPRR |
| | | KNPQEGLYNELQKDKMAEAYSEIGMKGERRRGKGHD |
| | | GLYQGLSTATKDTYDALHMQALPPR |
| 119 | CD84 hinge/TM-DAP10- | PVSNNSDSISARQLCADIAMGFRTHHTGLLSVLAME |
| | CD84ICD-CD3zeta-P2A- | FLLVLILSSVELELCARPRRSPAQEDGKVYINMPGR |
| | SAP in CAR18 | GRLFKRRQGRIFPEGSCLNTFTKNPYAASKKTIYTY |
| | | IMASRNTQPAESRIYDEILQSKVLPSKEEPVNTVYS |
| | | EVQFADKMGKASTQDSKPPGTSSYEIVISFGLLDPK |
| | | LCYLLDGILFIYGVILTALFLRVKFSRSADAPAYQQ |
| | | GQNQLYNELNLGRREEYDVLDKRRGRDPEMGGKPRR |
| | | KNPQEGLYNELQKDKMAEAYSEIGMKGERRRGKGHD |
| | | GLYQGLSTATKDTYDALHMQALPPRGSGATNFSLLK |
| | | QAGDVEENPGPMDAVAVYHGKISRETGEKLLLATGL |
| | | DGSYLLRDSESVPGVYCLCVLYHGYIYTYRVSQTET |
| | | GSWSAETAPGVHKRYFRKIKNLISAFQKPDQGIVIP |
| | | LQYPVEKKSSARSTQGTTGIREDPDVCLKAP |
| 120 | CD2 hinge/TM-DAP10- | IFDLKIQERVSKPKISWTCINTTLTCEVMNGTDPEL |
| | CD2ICD-CD3zeta in CAR19 | NLYQDGKHLKLSQRVITHKWTTSLSAKFKCTAGNKV |
| | | SKESSVEPVSCPEKGLDIYLIIGICGGGSLLMVEVA |
| | | LLVFYITLCARPRRSPAQEDGKVYINMPGRGKRKKQ |
| | | RSRRNDEELETRAHRVATEERGRKPHQIPASTPQNP |
| | | ATSQHPPPPPGHRSQAPSHRPPPPGHRVQHQPQKRP |
| | | PAPSGTQVHQQKGPPLPRPRVQPKPPHGAAENSLSP |
| | | SSNSFGLLDPKLCYLLDGILFIYGVILTALFLRVKE |
| | | SRSADAPAYQQGQNQLYNELNLGRREEYDVLDKRRG |
| | | RDPEMGGKPRRKNPQEGLYNELQKDKMAEAYSEIGM |
| | | KGERRRGKGHDGLYQGLSTATKDTYDALHMQALPPR |
| 121 | CD2 hinge/TM-DAP10- | IFDLKIQERVSKPKISWTCINTTLTCEVMNGTDPEL |
| | CD2ICD-CD3zeta-P2A-SAP | NLYQDGKHLKLSQRVITHKWTTSLSAKFKCTAGNKV |
| | in CAR20 | SKESSVEPVSCPEKGLDIYLIIGICGGGSLLMVEVA |
| | | LLVFYITLCARPRRSPAQEDGKVYINMPGRGKRKKQ |
| | | RSRRNDEELETRAHRVATEERGRKPHQIPASTPQNP |
| | | ATSQHPPPPPGHRSQAPSHRPPPPGHRVQHQPQKRP |
| | | PAPSGTQVHQQKGPPLPRPRVQPKPPHGAAENSLSP |
| | | SSNSFGLLDPKLCYLLDGILFIYGVILTALFLRVKE |
| | | SRSADAPAYQQGQNQLYNELNLGRREEYDVLDKRRG |
| | | RDPEMGGKPRRKNPQEGLYNELQKDKMAEAYSEIGM |
| | | KGERRRGKGHDGLYQGLSTATKDTYDALHMQALPPR |
| | | GSGATNFSLLKQAGDVEENPGPMDAVAVYHGKISRE |
| | | TGEKLLLATGLDGSYLLRDSESVPGVYCLCVLYHGY |
| | | IYTYRVSQTETGSWSAETAPGVHKRYFRKIKNLISA |
| | | FQKPDQGIVIPLQYPVEKKSSARSTQGTTGIREDPD |
| | | VCLKAP |
| 122 | CD28H hinge/TM-DAP10- | AVEIPELEEAEGNITRLFVDPDDPTQNRNRIASFPG |
| | CD28HICD-CD3zeta in | FLFVLLGVGSMGVAAIVWGAWLCARPRRSPAQEDGK |
| | CAR21 | VYINMPGRGFWGRRSCQQRDSGNSPGNAFYSNVLYR |
| | | PRGAPKKSEDCSGEGKDQRGQSIYSTSFPQPAPRQP |
| | | HLASRPCPSPRPCPSPRPGHPVSMVRVSPRPSPTQQ |
| | | PRPKGFPKVGEESFGLLDPKLCYLLDGILFIYGVIL |
| | | TALFLRVKESRSADAPAYQQGQNQLYNELNLGRREE |
| | | YDVLDKRRGRDPEMGGKPRRKNPQEGLYNELQKDKM |
| | | AEAYSEIGMKGERRRGKGHDGLYQGLSTATKDTYDA |
| | | LHMQALPPR |
| 123 | CD28H hinge/TM-DAP10- | AVEIPELEEAEGNITRLFVDPDDPTQNRNRIASFPG |
| | CD28HICD-CD3zeta-P2A- | FLFVLLGVGSMGVAAIVWGAWLCARPRRSPAQEDGK |
| | SAP in CAR22 | VYINMPGRGFWGRRSCQQRDSGNSPGNAFYSNVLYR |
| | | PRGAPKKSEDCSGEGKDQRGQSIYSTSFPQPAPRQP |
| | | HLASRPCPSPRPCPSPRPGHPVSMVRVSPRPSPTQQ |
| | | PRPKGFPKVGEESFGLLDPKLCYLLDGILFIYGVIL |
| | | TALFLRVKFSRSADAPAYQQGQNQLYNELNLGRREE |
| | | YDVLDKRRGRDPEMGGKPRRKNPQEGLYNELQKDKM |
| | | AEAYSEIGMKGERRRGKGHDGLYQGLSTATKDTYDA |
| | | LHMQALPPRGSGATNFSLLKQAGDVEENPGPMDAVA |
| | | VYHGKISRETGEKLLLATGLDGSYLLRDSESVPGVY |
| | | CLCVLYHGYIYTYRVSQTETGSWSAETAPGVHKRYF |
| | | RKIKNLISAFQKPDQGIVIPLQYPVEKKSSARSTQG |
| | | TTGIREDPDVCLKAP |
| 124 | DNAM1 hinge/TM-DAP10- | AEGKTDNQYTLFVAGGTVLLLLFVISITTIIVIFLL |
| | DNAMIICD-CD3zeta in | CARPRRSPAQEDGKVYINMPGRGNRRRRRERRDLFT |
| | CAR23 | ESWDTQKAPNNYRSPISTSQPTNQSMDDTREDIYVN |
| | | YPTFSRRPKTRVSFGLLDPKLCYLLDGILFIYGVIL |
| | | TALFLRVKESRSADAPAYQQGQNQLYNELNLGRREE |
| | | YDVLDKRRGRDPEMGGKPRRKNPQEGLYNELQKDKM |
| | | AEAYSEIGMKGERRRGKGHDGLYQGLSTATKDTYDA |
| | | LHMQALPPR |
| 125 | DNAM1 hinge/TM-DAP10- | AEGKTDNQYTLFVAGGTVLLLLFVISITTIIVIELL |
| | DNAMIICD-CD3zeta-P2A- | CARPRRSPAQEDGKVYINMPGRGNRRRRRERRDLFT |
| | SAP in CAR24 | ESWDTQKAPNNYRSPISTSQPTNQSMDDTREDIYVN |
| | | YPTFSRRPKTRVSFGLLDPKLCYLLDGILFIYGVIL |
| | | TALFLRVKESRSADAPAYQQGQNQLYNELNLGRREE |
| | | YDVLDKRRGRDPEMGGKPRRKNPQEGLYNELQKDKM |
| | | AEAYSEIGMKGERRRGKGHDGLYQGLSTATKDTYDA |
| | | LHMQALPPRGSGATNFSLLKQAGDVEENPGPMDAVA |
| | | VYHGKISRETGEKLLLATGLDGSYLLRDSESVPGVY |
| | | CLCVLYHGYIYTYRVSQTETGSWSAETAPGVHKRYF |
| | | RKIKNLISAFQKPDQGIVIPLQYPVEKKSSARSTQG |
| | | TTGIREDPDVCLKAP |
| 126 | CD28 hinge/TM-DAP10 | IHVKGKHLCPSPLFPGPSKPFWVLVVVGGVLACYSL |
| | ICD-CD28 ICD-CD3zeta in | LVTVAFIIFWVLCARPRRSPAQEDGKVYINMPGRGR |
| | CAR25 | SKRSRLLHSDYMNMTPRRPGPTRKHYQPYAPPRDFA |
| | | AYRSSFGLLDPKLCYLLDGILFIYGVILTALFLRVK |
| | | FSRSADAPAYQQGQNQLYNELNLGRREEYDVLDKRR |
| | | GRDPEMGGKPRRKNPQEGLYNELQKDKMAEAYSEIG |
| | | MKGERRRGKGHDGLYQGLSTATKDTYDALHMQALPP |
| | | R |
| 127 | CD28 hinge/TM-DAP10 | IHVKGKHLCPSPLFPGPSKPFWVLVVVGGVLACYSL |
| | ICD-CD28 ICD-CD3zeta- | LVTVAFIIFWVLCARPRRSPAQEDGKVYINMPGRGR |
| | P2A-SAP in CAR26 | SKRSRLLHSDYMNMTPRRPGPTRKHYQPYAPPRDFA |
| | | AYRSSFGLLDPKLCYLLDGILFIYGVILTALFLRVK |
| | | FSRSADAPAYQQGQNQLYNELNLGRREEYDVLDKRR |
| | | GRDPEMGGKPRRKNPQEGLYNELQKDKMAEAYSEIG |
| | | MKGERRRGKGHDGLYQGLSTATKDTYDALHMQALPP |
| | | RGSGATNFSLLKQAGDVEENPGPMDAVAVYHGKISR |
| | | ETGEKLLLATGLDGSYLLRDSESVPGVYCLCVLYHG |
| | | YIYTYRVSQTETGSWSAETAPGVHKRYFRKIKNLIS |
| | | AFQKPDQGIVIPLQYPVEKKSSARSTQGTTGIREDP |
| | | DVCLKAP |
| 128 | 4-1BB hinge/TM-DAP10 | SPADLSPGASSVTPPAPAREPGHSPQIISFFLALTS |
| | ICD-4-1BB ICD-CD3zeta in | TALLFLLFFLTLRESVVLCARPRRSPAQEDGKVYIN |
| | CAR27 | MPGRGKRGRKKLLYIFKQPFMRPVQTTQEEDGCSCR |
| | | FPEEEEGGCELSFGLLDPKLCYLLDGILFIYGVILT |
| | | ALFLRVKESRSADAPAYQQGQNQLYNELNLGRREEY |
| | | DVLDKRRGRDPEMGGKPRRKNPQEGLYNELQKDKMA |
| | | EAYSEIGMKGERRRGKGHDGLYQGLSTATKDTYDAL |
| | | HMQALPPR |
| 129 | 4-1BB hinge/TM-DAP10 | SPADLSPGASSVTPPAPAREPGHSPQIISFFLALTS |
| | ICD-4-1BB ICD-CD3zeta- | TALLFLLFFLTLRFSVVLCARPRRSPAQEDGKVYIN |
| | P2A-SAP in CAR28 | MPGRGKRGRKKLLYIFKQPFMRPVQTTQEEDGCSCR |
| | | FPEEEEGGCELSFGLLDPKLCYLLDGILFIYGVILT |
| | | ALFLRVKFSRSADAPAYQQGQNQLYNELNLGRREEY |
| | | DVLDKRRGRDPEMGGKPRRKNPQEGLYNELQKDKMA |
| | | EAYSEIGMKGERRRGKGHDGLYQGLSTATKDTYDAL |
| | | HMQALPPRGSGATNFSLLKQAGDVEENPGPMDAVAV |
| | | YHGKISRETGEKLLLATGLDGSYLLRDSESVPGVYC |
| | | LCVLYHGYIYTYRVSQTETGSWSAETAPGVHKRYFR |
| | | KIKNLISAFQKPDQGIVIPLQYPVEKKSSARSTQGT |
| | | TGIREDPDVCLKAP |
| 130 | OX40 hinge/TM-DAP10 | DRDPPATQPQETQGPPARPITVQPTEAWPRTSQGPS |
| | ICD-OX40 ICD-CD3zeta in | TRPVEVPGGRAVAAILGLGLVLGLLGPLAILLLCAR |
| | CAR29 | PRRSPAQEDGKVYINMPGRGALYLLRRDQRLPPDAH |
| | | KPPGGGSFRTPIQEEQADAHSTLAKISFGLLDPKLC |
| | | YLLDGILFIYGVILTALFLRVKFSRSADAPAYQQGQ |
| | | NQLYNELNLGRREEYDVLDKRRGRDPEMGGKPRRKN |
| | | PQEGLYNELQKDKMAEAYSEIGMKGERRRGKGHDGL |
| | | YQGLSTATKDTYDALHMQALPPR |
| 131 | OX40 hinge/TM-DAP10 | DRDPPATQPQETQGPPARPITVQPTEAWPRTSQGPS |
| | ICD-OX40 ICD-CD3zeta- | TRPVEVPGGRAVAAILGLGLVLGLLGPLAILLLCAR |
| | P2A-SAP in CAR30 | PRRSPAQEDGKVYINMPGRGALYLLRRDQRLPPDAH |
| | | KPPGGGSFRTPIQEEQADAHSTLAKISFGLLDPKLC |
| | | YLLDGILFIYGVILTALFLRVKFSRSADAPAYQQGQ |
| | | NQLYNELNLGRREEYDVLDKRRGRDPEMGGKPRRKN |
| | | PQEGLYNELQKDKMAEAYSEIGMKGERRRGKGHDGL |
| | | YQGLSTATKDTYDALHMQALPPRGSGATNFSLLKQA |
| | | GDVEENPGPMDAVAVYHGKISRETGEKLLLATGLDG |
| | | SYLLRDSESVPGVYCLCVLYHGYIYTYRVSQTETGS |
| | | WSAETAPGVHKRYFRKIKNLISAFQKPDQGIVIPLQ |
| | | YPVEKKSSARSTQGTTGIREDPDVCLKAP |
| 132 | KIR2DS1 hinge/TM-DAP10 | SFRDSPYEWSKSSDPLLVSVTGNPSNSWPSPTEPSS |
| | ICD-CD3zeta in CAR31 | ETGNPRHLHVLIGTSVVKIPFTILLFFLLLCARPRR |
| | | SPAQEDGKVYINMPGRGSFGLLDPKLCYLLDGILFI |
| | | YGVILTALFLRVKESRSADAPAYQQGQNQLYNELNL |
| | | GRREEYDVLDKRRGRDPEMGGKPRRKNPQEGLYNEL |
| | | QKDKMAEAYSEIGMKGERRRGKGHDGLYQGLSTATK |
| | | DTYDALHMQALPPR |
| 133 | KIR2DS1 hinge/TM-DAP10 | SFRDSPYEWSKSSDPLLVSVTGNPSNSWPSPTEPSS |
| | ICD-CD3zeta-P2A-SAP in | ETGNPRHLHVLIGTSVVKIPFTILLFFLLLCARPRR |
| | CAR32 | SPAQEDGKVYINMPGRGSFGLLDPKLCYLLDGILFI |
| | | YGVILTALFLRVKESRSADAPAYQQGQNQLYNELNL |
| | | GRREEYDVLDKRRGRDPEMGGKPRRKNPQEGLYNEL |
| | | QKDKMAEAYSEIGMKGERRRGKGHDGLYQGLSTATK |
| | | DTYDALHMQALPPRGSGATNFSLLKQAGDVEENPGP |
| | | MDAVAVYHGKISRETGEKLLLATGLDGSYLLRDSES |
| | | VPGVYCLCVLYHGYIYTYRVSQTETGSWSAETAPGV |
| | | HKRYFRKIKNLISAFQKPDQGIVIPLQYPVEKKSSA |
| | | RSTQGTTGIREDPDVCLKAP |
| 134 | KIR2DS2 hinge/TM-DAP10 | SFRDSPYEWSNSSDPLLVSVTGNPSNSWPSPTEPSS |
| | ICD-CD3zeta in CAR33 | KTGNPRHLHVLIGTSVVKIPFTILLFFLLLCARPRR |
| | | SPAQEDGKVYINMPGRGSFGLLDPKLCYLLDGILFI |
| | | YGVILTALFLRVKFSRSADAPAYQQGQNQLYNELNL |
| | | GRREEYDVLDKRRGRDPEMGGKPRRKNPQEGLYNEL |
| | | QKDKMAEAYSEIGMKGERRRGKGHDGLYQGLSTATK |
| | | DTYDALHMQALPPR |
| 135 | KIR2DS2 hinge/TM-DAP10 | SFRDSPYEWSNSSDPLLVSVTGNPSNSWPSPTEPSS |
| | ICD-CD3zeta-P2A-SAP in | KTGNPRHLHVLIGTSVVKIPFTILLFFLLLCARPRR |
| | CAR34 | SPAQEDGKVYINMPGRGSFGLLDPKLCYLLDGILFI |
| | | YGVILTALFLRVKFSRSADAPAYQQGQNQLYNELNL |
| | | GRREEYDVLDKRRGRDPEMGGKPRRKNPQEGLYNEL |
| | | QKDKMAEAYSEIGMKGERRRGKGHDGLYQGLSTATK |
| | | DTYDALHMQALPPRGSGATNESLLKQAGDVEENPGP |
| | | MDAVAVYHGKISRETGEKLLLATGLDGSYLLRDSES |
| | | VPGVYCLCVLYHGYIYTYRVSQTETGSWSAETAPGV |
| | | HKRYFRKIKNLISAFQKPDQGIVIPLQYPVEKKSSA |
| | | RSTQGTTGIREDPDVCLKAP |
| 136 | KIR2DS3 hinge/TM-DAP10 | SFHDSPYEWSKSSDPLLVSVTGNPSNSWPSPTEPSS |
| | ICD-CD3zeta in CAR35 | KTGNPRHLHVLIGTSVVKLPFTILLFELLLCARPRR |
| | | SPAQEDGKVYINMPGRGSFGLLDPKLCYLLDGILFI |
| | | YGVILTALFLRVKFSRSADAPAYQQGQNQLYNELNL |
| | | GRREEYDVLDKRRGRDPEMGGKPRRKNPQEGLYNEL |
| | | QKDKMAEAYSEIGMKGERRRGKGHDGLYQGLSTATK |
| | | DTYDALHMQALPPR |
| 137 | KIR2DS3 hinge/TM-DAP10 | SFHDSPYEWSKSSDPLLVSVTGNPSNSWPSPTEPSS |
| | ICD-CD3zeta-P2A-SAP in | KTGNPRHLHVLIGTSVVKLPFTILLFELLLCARPRR |
| | CAR36 | SPAQEDGKVYINMPGRGSFGLLDPKLCYLLDGILFI |
| | | YGVILTALFLRVKESRSADAPAYQQGQNQLYNELNL |
| | | GRREEYDVLDKRRGRDPEMGGKPRRKNPQEGLYNEL |
| | | QKDKMAEAYSEIGMKGERRRGKGHDGLYQGLSTATK |
| | | DTYDALHMQALPPRGSGATNFSLLKQAGDVEENPGP |
| | | MDAVAVYHGKISRETGEKLLLATGLDGSYLLRDSES |
| | | VPGVYCLCVLYHGYIYTYRVSQTETGSWSAETAPGV |
| | | HKRYFRKIKNLISAFQKPDQGIVIPLQYPVEKKSSA |
| | | RSTQGTTGIREDPDVCLKAP |
| 138 | KIR2DS4 hinge/TM-DAP10 | SFRDAPYEWSNSSDPLLVSVTGNPSNSWPSPTEPSS |
| | ICD-CD3zeta in CAR37 | KTGNPRHLHVLIGTSVVKIPFTILLFFLLLCARPRR |
| | | SPAQEDGKVYINMPGRGSFGLLDPKLCYLLDGILFI |
| | | YGVILTALFLRVKESRSADAPAYQQGQNQLYNELNL |
| | | GRREEYDVLDKRRGRDPEMGGKPRRKNPQEGLYNEL |
| | | QKDKMAEAYSEIGMKGERRRGKGHDGLYQGLSTATK |
| | | DTYDALHMQALPPR |
| 139 | KIR2DS4 hinge/TM-DAP10 | SFRDAPYEWSNSSDPLLVSVTGNPSNSWPSPTEPSS |
| | ICD-CD3zeta-P2A-SAP in | KTGNPRHLHVLIGTSVVKIPFTILLFFLLLCARPRR |
| | CAR38 | SPAQEDGKVYINMPGRGSFGLLDPKLCYLLDGILFI |
| | | YGVILTALFLRVKESRSADAPAYQQGQNQLYNELNL |
| | | GRREEYDVLDKRRGRDPEMGGKPRRKNPQEGLYNEL |
| | | QKDKMAEAYSEIGMKGERRRGKGHDGLYQGLSTATK |
| | | DTYDALHMQALPPRGSGATNFSLLKQAGDVEENPGP |
| | | MDAVAVYHGKISRETGEKLLLATGLDGSYLLRDSES |
| | | VPGVYCLCVLYHGYIYTYRVSQTETGSWSAETAPGV |
| | | HKRYFRKIKNLISAFQKPDQGIVIPLQYPVEKKSSA |
| | | RSTQGTTGIREDPDVCLKAP |
| 140 | KIR2DS5 hinge/TM-DAP10 | SFRDSPYEWSKSSDPLLVSVTGNSSNSWPSPTEPSS |
| | ICD-CD3zeta in CAR39 | ETGNPRHLHVLIGTSVVKLPFTILLFFLLLCARPRR |
| | | SPAQEDGKVYINMPGRGSFGLLDPKLCYLLDGILFI |
| | | YGVILTALFLRVKFSRSADAPAYQQGQNQLYNELNL |
| | | GRREEYDVLDKRRGRDPEMGGKPRRKNPQEGLYNEL |
| | | QKDKMAEAYSEIGMKGERRRGKGHDGLYQGLSTATK |
| | | DTYDALHMQALPPR |
| 141 | KIR2DS5 hinge/TM-DAP10 | SFRDSPYEWSKSSDPLLVSVTGNSSNSWPSPTEPSS |
| | ICD-CD3zeta-P2A-SAP in | ETGNPRHLHVLIGTSVVKLPFTILLFFLLLCARPRR |
| | CAR40 | SPAQEDGKVYINMPGRGSFGLLDPKLCYLLDGILFI |
| | | YGVILTALFLRVKESRSADAPAYQQGQNQLYNELNL |
| | | GRREEYDVLDKRRGRDPEMGGKPRRKNPQEGLYNEL |
| | | QKDKMAEAYSEIGMKGERRRGKGHDGLYQGLSTATK |
| | | DTYDALHMQALPPRGSGATNFSLLKQAGDVEENPGP |
| | | MDAVAVYHGKISRETGEKLLLATGLDGSYLLRDSES |
| | | VPGVYCLCVLYHGYIYTYRVSQTETGSWSAETAPGV |
| | | HKRYFRKIKNLISAFQKPDQGIVIPLQYPVEKKSSA |
| | | RSTQGTTGIREDPDVCLKAP |
| 142 | NKG2C hinge/TM-DAP10 | IPFLEQNNFSPNTRTQKARHCGHLTAEVLGIICIVL |
| | ICD-CD3zeta in CAR41 | MATVLKTIVLLCARPRRSPAQEDGKVYINMPGRGSF |
| | | GLLDPKLCYLLDGILFIYGVILTALFLRVKESRSAD |
| | | APAYQQGQNQLYNELNLGRREEYDVLDKRRGRDPEM |
| | | GGKPRRKNPQEGLYNELQKDKMAEAYSEIGMKGERR |
| | | RGKGHDGLYQGLSTATKDTYDALHMQALPPR |
| 143 | NKG2C hinge/TM-DAP10 | IPFLEQNNFSPNTRTQKARHCGHLTAEVLGIICIVL |
| | ICD-CD3zeta-P2A-SAP in | MATVLKTIVLLCARPRRSPAQEDGKVYINMPGRGSF |
| | CAR42 | GLLDPKLCYLLDGILFIYGVILTALFLRVKFSRSAD |
| | | APAYQQGQNQLYNELNLGRREEYDVLDKRRGRDPEM |
| | | GGKPRRKNPQEGLYNELQKDKMAEAYSEIGMKGERR |
| | | RGKGHDGLYQGLSTATKDTYDALHMQALPPRGSGAT |
| | | NFSLLKQAGDVEENPGPMDAVAVYHGKISRETGEKL |
| | | LLATGLDGSYLLRDSESVPGVYCLCVLYHGYIYTYR |
| | | VSQTETGSWSAETAPGVHKRYFRKIKNLISAFQKPD |
| | | QGIVIPLQYPVEKKSSARSTQGTTGIREDPDVCLKA |
| | | P |
| 144 | NKG2E hinge/TM-DAP10 | IPFLEQNNSSPNARTQKARHCGHLTAEVLGIICIVL |
| | ICD-CD3zeta in CAR43 | MATVLKTIVLLCARPRRSPAQEDGKVYINMPGRGSF |
| | | GLLDPKLCYLLDGILFIYGVILTALFLRVKESRSAD |
| | | APAYQQGQNQLYNELNLGRREEYDVLDKRRGRDPEM |
| | | GGKPRRKNPQEGLYNELQKDKMAEAYSEIGMKGERR |
| | | RGKGHDGLYQGLSTATKDTYDALHMQALPPR |
| 145 | NKG2E hinge/TM-DAP10 | IPFLEQNNSSPNARTQKARHCGHLTAEVLGIICIVL |
| | ICD-CD3zeta-P2A-SAP in | MATVLKTIVLLCARPRRSPAQEDGKVYINMPGRGSF |
| | CAR44 | GLLDPKLCYLLDGILFIYGVILTALFLRVKESRSAD |
| | | APAYQQGQNQLYNELNLGRREEYDVLDKRRGRDPEM |
| | | GGKPRRKNPQEGLYNELQKDKMAEAYSEIGMKGERR |
| | | RGKGHDGLYQGLSTATKDTYDALHMQALPPRGSGAT |
| | | NFSLLKQAGDVEENPGPMDAVAVYHGKISRETGEKL |
| | | LLATGLDGSYLLRDSESVPGVYCLCVLYHGYIYTYR |
| | | VSQTETGSWSAETAPGVHKRYFRKIKNLISAFQKPD |
| | | QGIVIPLQYPVEKKSSARSTQGTTGIREDPDVCLKA |
| | | P |
| 146 | Exemplary peptide linker | (GS)ₙ, n is an integer including, |
| | | e.g., 1, 2, 3, 4, 5, and 6 |
| 147 | Exemplary peptide linker | (GSGGS)ₙ, n is an integer including, |
| | | e.g., 1, 2, 3, 4, 5, and 6 |
| 148 | Exemplary peptide linker | (GGGS)ₙ, n is an integer including, |
| | | e.g., 1, 2, 3, 4, 5, and 6 |
| 149 | Exemplary peptide linker | (GGGGS)ₙ, n is an integer including, |
| | | e.g., 1, 2, 3, 4, 5, and 6 |
| 150 | Exemplary peptide linker | GS |
| 151 | Exemplary peptide linker | GSGS |
| 152 | Exemplary peptide linker | GSGSGS |
| 153 | Exemplary peptide linker | GGGS |
| 154 | Exemplary peptide linker | GGGSGGGS |
| 155 | Exemplary peptide linker | GSGGS |
| 156 | Exemplary peptide linker | GSGGSGSGGS |
| 157 | Exemplary peptide linker | GSGGGS |
| 158 | Exemplary peptide linker | GGGGS |
| 159 | Exemplary peptide linker | GGGGSGGGGS |
| 160 | Exemplary peptide linker | GGRR |
| 161 | Exemplary peptide linker | GGGGSGGGGSGGGGGGSGSGGGGS |
| 162 | Exemplary peptide linker | GGRRGGGS |
| 163 | Anti-CD19 scFv | EVKLQQSGAELVRPGSSVKISCKASGYAFSSYWMNW |
| | | VKQRPGQGLEWIGQIYPGDGDTNYNGKFKGQATLTA |
| | | DKSSSTAYMQLSGLTSEDSAVYFCARKTISSVVDFY |
| | | FDYWGQGTTVTVSSGGGGSGGGGSGGGGSDIELTQS |
| | | PKFMSTSVGDRVSVTCKASQNVGTNVAWYQQKPGQS |
| | | PKPLIYSATYRNSGVPDRFTGSGSGTDFTLTITNVQ |
| | | SKDLADYFCQQYNRYPYTSGGGTKLEIK |
| 164 | VH CDR1 of anti-CD19 scFv | GYAFSSYW |
| | as determined by software | |
| | Gencious Biologics | |
| 165 | VH CDR2 of anti-CD19 scFv | IYPGDGDT |
| | as determined by software | |
| | Geneious Biologics | |
| 166 | VH CDR3 of anti-CD19 scFv | ARKTISSVVDFYFDY |
| | as determined by software | |
| | Geneious Biologics | |
| 167 | VL CDR1 of anti-CD19 scFv | QNVGTN |
| | as determined by software | |
| | Geneious Biologics | |
| 168 | VL CDR2 of anti-CD19 scFv | SAT |
| | as determined by software | |
| | Geneious Biologics | |
| 169 | VL CDR3 of anti-CD19 scFv | QQYNRYPYT |
| | as determined by software | |
| | Geneious Biologics | |
| 170 | Anti-GPC3 scFv | QVQLQQSGAELVRPGASVKLSCKASGYTFTDYEMHW |
| | | VKQTPVHGLKWIGALDPKTGDTAYSQKFKGKATLTA |
| | | DKSSSTAYMELRSLTSEDSAVYYCTRFYSYTYWGQG |
| | | TLVTVSGGGGSGGGGSGGGGSDVVMTQTPLSLPVSL |
| | | GDQASISCRSSQSLVHSNGNTYLHWYLQKPGQSPKL |
| | | LIYKVSNRFSGVPDRESGSGSGTDETLKISRVEAED |
| | | LGVYFCSQNTHVPPTFGSGTKLEIKR |
| 171 | VH CDR1 of anti-GPC3 scFv | GYTFTDYE |
| | as determined by software | |
| | Geneious Biologics | |
| 172 | VH CDR2 of anti-GPC3 scFv | LDPKTGDT |
| | as determined by software | |
| | Geneious Biologics | |
| 173 | VH CDR3 of anti-GPC3 scFv | TRFYSYTY |
| | as determined by software | |
| | Geneious Biologics | |
| 174 | VL CDR1 of anti-GPC3 scFv | QSLVHSNGNTY |
| | as determined by software | |
| | Geneious Biologics | |
| 175 | VL CDR2 of anti-GPC3 scFv | KVS |
| | as determined by software | |
| | Geneious Biologics | |
| 176 | VL CDR3 of anti-GPC3 scFv | SQNTHVPPT |
| | as determined by software | |
| | Geneious Biologics | |
| 177 | Reversed sequence of DAP10 | GRGPMNIYVKGDEDQAPSRRPRACL |
| | ICD | |
| 178 | Reversed sequence of SAP | PAKLCVDPDERIGTTGQTSRASSKKEVPYQLPIVIG |
| | | QDPKQFASILNKIKRFYRKHVGPATEASWSGTETQS |
| | | VRYTYIYGHYLVCLCYVGPVSESDRLLYSGDLGTAL |
| | | LLKEGTERSIKGHYVAVADM |
| 179 | Reversed sequence of EAT2 | PLVDVYDSNSNVFTELELKLGRWRLSPSTRKIPKLL |
| | | HVVMGQNPKEFKSILEKLSPFVQKPSGEATQIRYYG |
| | | HKERFIRYTYVINKFSVCLCLVGPISESDRLLENGD |
| | | VGEKLLLTECDQKTLRGHYYPLDM |

1. A chimeric antigen receptor (CAR), comprising:
(i) an extracellular antigen binding domain;
(ii) a transmembrane domain; and
(iii) an intracellular signaling domain comprising an intracellular domain derived from a first polypeptide,
wherein the intracellular domain derived from the first polypeptide is linked to the C-terminus of the transmembrane domain directly or via a peptide linker, and
wherein the first polypeptide is selected from: DAP10, DAP12 and DNAM1;
wherein the intracellular signaling domain of the CAR further comprises an intracellular domain derived from CD3zeta.

2. (canceled)

3. The CAR of claim 1, wherein the transmembrane domain of the CAR comprises a transmembrane domain derived from a second polypeptide, wherein the second polypeptide is different from the first polypeptide, and wherein the intracellular domain derived from the first polypeptide is linked to the C-terminus of the transmembrane domain derived from the second polypeptide directly or via a peptide linker.

4. The CAR of claim 3, wherein the intracellular signaling domain of the CAR further comprises a intracellular domain derived from a third polypeptide, wherein the third polypeptide is different from the first polypeptide;
wherein the second polypeptide and the third polypeptide are respectively selected from: a signaling lymphocytic activation molecule family (SLAMF) receptor, CD2, CD28H, DNAM1, CD28, 4-1BB, OX40, KIR-S, NKG2C, and NKG2E.

5. (canceled)

6. The CAR of claim 4, wherein the CAR further comprises a hinge domain between the C-terminus of the extracellular antigen binding domain and the N-terminus of the transmembrane domain;
wherein the hinge domain of the CAR comprises a hinge domain derived from the second polypeptide.

7-8. (canceled)

9. The CAR of claim 4, wherein the SLAMF receptor is selected from: SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), and SLAMF9 (CD84H1, SF2001); and wherein the KIR-S is selected from KIR2DS1, KIR2DS2, KIR2DS3, KIR2DS4, KIR2DS5 and KIR3DS1.

10-11. (canceled)

12. The CAR of claim 4, wherein the intracellular signaling domain of the CAR further comprises an intracellular domain selected from: an intracellular domain derived from SLAM-associated protein (SAP), and an intracellular domain derived from Ewing's sarcoma-activated transcript-2 (EAT2); and the intracellular domain derived from CD3zeta is linked to said intracellular domain selected directly or via a peptide linker.

13-14. (canceled)

15. The CAR of claim 12, wherein the intracellular domain derived from CD3zeta is linked to said intracellular domain selected via a peptide linker selected from: P2A, T2A, and E2A.

16. The CAR of claim 1, wherein the extracellular antigen binding domain of the CAR comprises an scFv, and wherein the extracellular antigen binding domain of the CAR binds to a tumor antigen or a fragment thereof.

17-21. (canceled)

22. The CAR of claim 1 comprising an amino acid sequence at least 90% identical to an amino acid sequence selected from SEQ ID NO:20, SEQ ID NO:86, SEQ ID NO:87, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, and SEQ ID NO:145.

23. The CAR of 12, from N-terminus to C-terminus, comprising:
(i) an extracellular antigen binding domain;
(ii) a transmembrane domain comprising an transmembrane domain derived from the second polypeptide;
(iii) an intracellular signaling domain sequentially comprising
(iii)-(a) an intracellular domain derived from DAP10;
(iii)-(b) an intracellular domain derived from CD3zeta; and
(iii)-(c) an intracellular domain derived from SAP or an intracellular domain derived from EAT2,
wherein the intracellular domains within the intracellular signaling domain are linked to each other directly or via one or more peptide linkers, and
wherein the extracellular antigen binding domain, the transmembrane domain, and the intracellular domain are linked to each other directly or via one or more peptide linkers.

24. An engineered immune cell comprising the CAR of claim 1, wherein the engineered immune cell is selected from: an NK cell, an NKT cell, and a T cell.

25-31. (canceled)

32. The engineered immune cell of claim 24, wherein the transmembrane domain of the CAR comprises a transmembrane domain derived from a second polypeptide, wherein the second polypeptide is different from the first polypeptide, and wherein the intracellular domain derived from the first polypeptide is linked to the C-terminus of the transmembrane domain derived from the second polypeptide directly or via a peptide linker;
and wherein the intracellular signaling domain of the CAR further comprises a intracellular domain derived from a third polypeptide, wherein the third polypeptide is different from the first polypeptide;
wherein the second polypeptide and the third polypeptide are respectively selected from: a signaling lymphocytic activation molecule family (SLAMF) receptor, CD2, CD28H, DNAM1, CD28, 4-1BB, OX40, KIR-S, NKG2C, and NKG2E.

33. The engineered immune cell of claim 32, wherein the SLAMF receptor is selected from: SLAMF4 (CD244, 2B4), SLAMF3 (CD229, Ly9), SLAMF6 (CD352, NTB-A, SF2000), SLAMF5 (CD84), SLAMF7 (CD319, CRACC, CS1), SLAMF1 (CD150), SLAMF2 (CD48, FimH), SLAMF8 (CD353), and SLAMF9 (CD84H1, SF2001); and wherein the KIR-S is selected from: KIR2DS1, KIR2DS2, KIR2DS3, KIR2DS4, KIR2DS5 and KIR3DS1.

34-35. (canceled)

36. The engineered immune cell of claim 24, wherein the intracellular signaling domain of the CAR further comprises an intracellular domain selected from: an intracellular domain derived from SLAM-associated protein (SAP) and an intracellular domain derived from EAT2, and wherein the intracellular domain derived from CD3zeta is linked to said intracellular domain selected directly or via a peptide linker.

37. The engineered immune cell of claim 36, wherein the intracellular domain derived from CD3zeta is linked to said intracellular domain selected via a peptide linker selected from: P2A, T2A, or E2A.

38-42. (canceled)

43. The engineered immune cell of claim 24, wherein the CAR comprises an amino acid sequence at least 90% identical to an amino acid sequence selected from: SEQ ID NO:20, SEQ ID NO:86, SEQ ID NO:87, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, or SEQ ID NO:145.

44-51. (canceled)

52. A pharmaceutical composition comprising
(i) the CAR of claim 1; and
(ii) a pharmaceutically acceptable excipient.

53-55. (canceled)

56. The pharmaceutical composition of claim 52, wherein the CAR is expressed on an engineered immune cell, and the engineered immune cell is selected from: an NK cell, an NKT cell, and a T cell.

57. The pharmaceutical composition of claim 52, wherein the CAR comprises an amino acid sequence at least 90% identical to an amino acid sequence selected from: SEQ ID NO:20, SEQ ID NO:86, SEQ ID NO:87, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, or SEQ ID NO:145.

58. The pharmaceutical composition of claim 52, for use in treating cancer.


SEQUENCE LISTING (179 sequences)

SEQ ID NO: 1 - protein, 22 aa, synthetic construct
  source 1..22: note = P2A
GSGATNFSLLKQAGDVEENPGP

SEQ ID NO: 2 - protein, 21 aa, synthetic construct
  source 1..21: note = T2A
GSGEGRGSLLRCGDVEENPGP

SEQ ID NO: 3 - protein, 23 aa, synthetic construct
  source 1..23: note = E2A
GSGQCTNYALLKLAGDVESNPGP

SEQ ID NO: 4 - protein, 21 aa, synthetic construct
  source 1..21: note = CD8 signal peptide for CAR expression
MALPVTALLLPLALLLHAARP

SEQ ID NO: 5 - protein, 49 aa, synthetic construct
  source 1..49: note = CD8 hinge
PAKPTTTPAPRPPTPAPTIASQPLSLRPEACRPAAGGAVHTRGLDFACD

SEQ ID NO: 6 - protein, 88 aa, synthetic construct
  source 1..88: note = CD28 hinge, TM and ICD
IHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 7 - protein, 20 aa, synthetic construct
  source 1..20: note = CD28 hinge
IHVKGKHLCPSPLFPGPSKP

SEQ ID NO: 8 - protein, 27 aa, synthetic construct
  source 1..27: note = CD28 TM
FWVLVVVGGVLACYSLLVTVAFIIFWV

SEQ ID NO: 9 - protein, 41 aa, synthetic construct
  source 1..41: note = CD28 ICD
RSKRSRLLHSDYMNMTPRRPGPTRKHYQPYAPPRDFAAYRS

SEQ ID NO: 10 - protein, 95 aa, synthetic construct
  source 1..95: note = 4-1BB hinge, TM, and ICD
SPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 11 - protein, 26 aa, synthetic construct
  source 1..26: note = 4-1BB hinge
SPADLSPGASSVTPPAPAREPGHSPQ

SEQ ID NO: 12 - protein, 27 aa, synthetic construct
  source 1..27: note = 4-1BB TM
IISFFLALTSTALLFLLFFLTLRFSVV

SEQ ID NO: 13 - protein, 42 aa, synthetic construct
  source 1..42: note = 4-1BB ICD
KRGRKKLLYIFKQPFMRPVQTTQEEDGCSCRFPEEEEGGCEL

SEQ ID NO: 14 - protein, 110 aa, synthetic construct
  source 1..110: note = OX40 hinge, TM, and ICD
DRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 15 - protein, 47 aa, synthetic construct
  source 1..47: note = OX40 hinge
DRDPPATQPQETQGPPARPITVQPTEAWPRTSQGPSTRPVEVPGGRA

SEQ ID NO: 16 - protein, 21 aa, synthetic construct
  source 1..21: note = OX40 TM
VAAILGLGLVLGLLGPLAILL

SEQ ID NO: 17 - protein, 42 aa, synthetic construct
  source 1..42: note = OX40 ICD
ALYLLRRDQRLPPDAHKPPGGGSFRTPIQEEQADAHSTLAKI

SEQ ID NO: 18 - protein, 21 aa, synthetic construct
  source 1..21: note = NKG2D TM
PFFFCCFIAVAMGIRFIIMVT

SEQ ID NO: 19 - protein, 141 aa, synthetic construct
  source 1..141: note = CD3zeta ICD
SFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 24 aa, synthetic construct
  source 1..24: note = DAP10 ICD
LCARPRRSPAQEDGKVYINMPGRG

SEQ ID NO: 21 - protein, 52 aa, synthetic construct
  source 1..52: note = DAP12 ICD
YFLGRLVPRGRGAAEAATRKQRITETESPYQELQGQRSDVYSDLNTQRPYYK

SEQ ID NO: 22 - protein, 243 aa, synthetic construct
  source 1..243: note = 2B4 hinge, TM and ICD
SLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 23 - protein, 102 aa, synthetic construct
  source 1..102: note = 2B4 hinge
SLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWP

SEQ ID NO: 24 - protein, 21 aa, synthetic construct
  source 1..21: note = 2B4 TM
FLVIIVILSALFLGTLACFCV

SEQ ID NO: 25 - protein, 120 aa, synthetic construct
  source 1..120: note = 2B4 ICD
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 26 - protein, 224 aa, synthetic construct
  source 1..224: note = CD229 hinge, TM and ICD
PVSRSSHQFLSENICSGPERNTKLWIGLFLMVCLLCVGIFSWCIWKRKGRCSVPAFCSSQAEAPADTPEPTAGHTLYSVLSQGYEKLDTPLRPARQQPTPTSDSSSDSNLTTEEDEDRPEVHKPISGRYEVFDQVTQEGAGHDPAPEGQADYDPVTPYVTEVESVVGENTMYAQVFNLQGKTPVSQKEESSATIYCSIRKPQVVPPPQQNDLEIPESPTYENFT

SEQ ID NO: 27 - protein, 23 aa, synthetic construct
  source 1..23: note = CD229 hinge
PVSRSSHQFLSENICSGPERNTK

SEQ ID NO: 28 - protein, 22 aa, synthetic construct
  source 1..22: note = CD229 TM
LWIGLFLMVCLLCVGIFSWCIW

SEQ ID NO: 29 - protein, 179 aa, synthetic construct
  source 1..179: note = CD229 ICD
KRKGRCSVPAFCSSQAEAPADTPEPTAGHTLYSVLSQGYEKLDTPLRPARQQPTPTSDSSSDSNLTTEEDEDRPEVHKPISGRYEVFDQVTQEGAGHDPAPEGQADYDPVTPYVTEVESVVGENTMYAQVFNLQGKTPVSQKEESSATIYCSIRKPQVVPPPQQNDLEIPESPTYENFT

SEQ ID NO: 30 - protein, 206 aa, synthetic construct
  source 1..206: note = NTB-A hinge, TM and ICD
LRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTKMILFMVSGICIVFGFIILLLLVLRKRRDSLSLSTQRTQGPAESARNLEYVSVSPTNNTVYASVTHSNRETEIWTPRENDTITIYSTINHSKESKPTFSRATALDNVV

SEQ ID NO: 31 - protein, 100 aa, synthetic construct
  source 1..100: note = NTB-A hinge
LRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTKM

SEQ ID NO: 32 - protein, 21 aa, synthetic construct
  source 1..21: note = NTB-A TM
ILFMVSGICIVFGFIILLLLV

SEQ ID NO: 33 - protein, 85 aa, synthetic construct
  source 1..85: note = NTB-A ICD
LRKRRDSLSLSTQRTQGPAESARNLEYVSVSPTNNTVYASVTHSNRETEIWTPRENDTITIYSTINHSKESKPTFSRATALDNVV

SEQ ID NO: 34 - protein, 232 aa, synthetic construct
  source 1..232: note = CRACC hinge, TM and ICD
YVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 35 - protein, 123 aa, synthetic construct
  source 1..123: note = CRACC hinge
YVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSM

SEQ ID NO: 36 - protein, 21 aa, synthetic construct
  source 1..21: note = CRACC TM
VLLCLLLVPLLLSLFVLGLFL

SEQ ID NO: 37 - protein, 88 aa, synthetic construct
  source 1..88: note = CRACC ICD
WFLKRERQEEYIEEKKRVDICRETPNICPHSGENTEYDTIPHTNRTILKEDPANTVYSTVEIPKKMENPHSLLTMPDTPRLFAYENVI

SEQ ID NO: 38 - protein, 148 aa, synthetic construct
  source 1..148: note = CD84 hinge, TM and ICD
PVSNNSDSISARQLCADIAMGFRTHHTGLLSVLAMFFLLVLILSSVFLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 39 - protein, 28 aa, synthetic construct
  source 1..28: note = CD84 hinge
PVSNNSDSISARQLCADIAMGFRTHHTG

SEQ ID NO: 40 - protein, 21 aa, synthetic construct
  source 1..21: note = CD84 TM
LLSVLAMFFLLVLILSSVFLF

SEQ ID NO: 41 - protein, 99 aa, synthetic construct
  source 1..99: note = CD84 ICD
RLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 42 - protein, 231 aa, synthetic construct
  source 1..231: note = CD2 hinge, TM and ICD
IFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 43 - protein, 89 aa, synthetic construct
  source 1..89: note = CD2 hinge
IFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 44 - protein, 26 aa, synthetic construct
  source 1..26: note = CD2 TM
IYLIIGICGGGSLLMVFVALLVFYIT

SEQ ID NO: 45 - protein, 116 aa, synthetic construct
  source 1..116: note = CD2 ICD
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 46 - protein, 168 aa, synthetic construct
  source 1..168: note = CD28H hinge, TM and ICD
AVEIPELEEAEGNITRLFVDPDDPTQNRNRIASFPGFLFVLLGVGSMGVAAIVWGAWFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 47 - protein, 36 aa, synthetic construct
  source 1..36: note = CD28H hinge
AVEIPELEEAEGNITRLFVDPDDPTQNRNRIASFPG

SEQ ID NO: 48 - protein, 21 aa, synthetic construct
  source 1..21: note = CD28H TM
FLFVLLGVGSMGVAAIVWGAW

SEQ ID NO: 49 - protein, 111 aa, synthetic construct
  source 1..111: note = CD28H ICD
FWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 50 - protein, 96 aa, synthetic construct
  source 1..96: note = DNAM1 hinge, TM and ICD
AEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 51 - protein, 14 aa, synthetic construct
  source 1..14: note = DNAM1 hinge
AEGKTDNQYTLFVA

SEQ ID NO: 52 - protein, 21 aa, synthetic construct
  source 1..21: note = DNAM1 TM
GGTVLLLLFVISITTIIVIFL

SEQ ID NO: 53 - protein, 61 aa, synthetic construct
  source 1..61: note = DNAM1 ICD
NRRRRRERRDLFTESWDTQKAPNNYRSPISTSQPTNQSMDDTREDIYVNYPTFSRRPKTRV

SEQ ID NO: 54 - protein, 104 aa, synthetic construct
  source 1..104: note = KIR2DS1 hinge, TM, ICD
SFRDSPYEWSKSSDPLLVSVTGNPSNSWPSPTEPSSETGNPRHLHVLIGTSVVKIPFTILLFFLLHRWCSDKKNAAVMDQEPAGNRTVNSEDSDEQDHQEVSYA

SEQ ID NO: 55 - protein, 45 aa, synthetic construct
  source 1..45: note = KIR2DS1 hinge
SFRDSPYEWSKSSDPLLVSVTGNPSNSWPSPTEPSSETGNPRHLH

SEQ ID NO: 56 - protein, 20 aa, synthetic construct
  source 1..20: note = KIR2DS1 TM
VLIGTSVVKIPFTILLFFLL

SEQ ID NO: 57 - protein, 39 aa, synthetic construct
  source 1..39: note = KIR2DS1 ICD
HRWCSDKKNAAVMDQEPAGNRTVNSEDSDEQDHQEVSYA

SEQ ID NO: 58 - protein, 104 aa, synthetic construct
  source 1..104: note = KIR2DS2 hinge, TM, ICD
SFRDSPYEWSNSSDPLLVSVTGNPSNSWPSPTEPSSKTGNPRHLHVLIGTSVVKIPFTILLFFLLHRWCSNKKNAAVMDQEPAGNRTVNSEDSDEQDHQEVSYA

SEQ ID NO: 59 - protein, 45 aa, synthetic construct
  source 1..45: note = KIR2DS2 hinge
SFRDSPYEWSNSSDPLLVSVTGNPSNSWPSPTEPSSKTGNPRHLH

SEQ ID NO: 60 - protein, 20 aa, synthetic construct
  source 1..20: note = KIR2DS2 TM
VLIGTSVVKIPFTILLFFLL

SEQ ID NO: 61 - protein, 39 aa, synthetic construct
  source 1..39: note = KIR2DS2 ICD
HRWCSNKKNAAVMDQEPAGNRTVNSEDSDEQDHQEVSYA

SEQ ID NO: 62 - protein, 104 aa, synthetic construct
  source 1..104: note = KIR2DS3 hinge, TM, ICD
SFHDSPYEWSKSSDPLLVSVTGNPSNSWPSPTEPSSKTGNPRHLHVLIGTSVVKLPFTILLFFLLHRWCSDKKNASVMDQGPAGNRTVNREDSDEQDHQEVSYA

SEQ ID NO: 63 - protein, 45 aa, synthetic construct
  source 1..45: note = KIR2DS3 hinge
SFHDSPYEWSKSSDPLLVSVTGNPSNSWPSPTEPSSKTGNPRHLH

SEQ ID NO: 64 - protein, 20 aa, synthetic construct
  source 1..20: note = KIR2DS3 TM
VLIGTSVVKLPFTILLFFLL

SEQ ID NO: 65 - protein, 39 aa, synthetic construct
  source 1..39: note = KIR2DS3 ICD
HRWCSDKKNASVMDQGPAGNRTVNREDSDEQDHQEVSYA

SEQ ID NO: 66 - protein, 104 aa, synthetic construct
  source 1..104: note = KIR2DS4 hinge, TM, ICD
SFRDAPYEWSNSSDPLLVSVTGNPSNSWPSPTEPSSKTGNPRHLHVLIGTSVVKIPFTILLFFLLHRWCSDKKNAAVMDQEPAGNRTVNSEDSDEQDHQEVSYA

SEQ ID NO: 67 - protein, 45 aa, synthetic construct
  source 1..45: note = KIR2DS4 hinge
SFRDAPYEWSNSSDPLLVSVTGNPSNSWPSPTEPSSKTGNPRHLH

SEQ ID NO: 68 - protein, 20 aa, synthetic construct
  source 1..20: note = KIR2DS4 TM
VLIGTSVVKIPFTILLFFLL

SEQ ID NO: 69 - protein, 39 aa, synthetic construct; identical to SEQ ID NO: 57
  source 1..39: note = KIR2DS4 ICD

SEQ ID NO: 70 - protein, 104 aa, synthetic construct
  source 1..104: note = KIR2DS5 hinge, TM, ICD
SFRDSPYEWSKSSDPLLVSVTGNSSNSWPSPTEPSSETGNPRHLHVLIGTSVVKLPFTILLFFLLHRWCSNKKNASVMDQGPAGNRTVNREDSDEQDHQEVSYA

SEQ ID NO: 71 - protein, 45 aa, synthetic construct
  source 1..45: note = KIR2DS5 hinge
SFRDSPYEWSKSSDPLLVSVTGNSSNSWPSPTEPSSETGNPRHLH

SEQ ID NO: 72 - protein, 20 aa, synthetic construct
  source 1..20: note = KIR2DS5 TM
VLIGTSVVKLPFTILLFFLL

SEQ ID NO: 73 - protein, 39 aa, synthetic construct
  source 1..39: note = KIR2DS5 ICD
HRWCSNKKNASVMDQGPAGNRTVNREDSDEQDHQEVSYA

SEQ ID NO: 74 - protein, 87 aa, synthetic construct
  source 1..87: note = KIR3DS1 hinge, TM, ICD
SFRHSPYEWSDPSDPLLVSVTGNPSSSWPSPTEPSSKSGNLRHLHILIGTSVVKIPFTILLFFLLHRWCSNKKKCCCNGPRACREQK

SEQ ID NO: 75 - protein, 45 aa, synthetic construct
  source 1..45: note = KIR3DS1 hinge
SFRHSPYEWSDPSDPLLVSVTGNPSSSWPSPTEPSSKSGNLRHLH

SEQ ID NO: 76 - protein, 20 aa, synthetic construct
  source 1..20: note = KIR3DS1 TM
ILIGTSVVKIPFTILLFFLL

SEQ ID NO: 77 - protein, 22 aa, synthetic construct
  source 1..22: note = KIR3DS1 ICD
HRWCSNKKKCCCNGPRACREQK

SEQ ID NO: 78 - protein, 116 aa, synthetic construct
  source 1..116: note = NKG2C ICD, TM, hinge
MNKQRGTFSEVSLAQDPKRQQRKPKGNKSSISGTEQEIFQVELNLQNPSLNHQGIDKIYDCQGLLPPPEKLTAEVLGIICIVLMATVLKTIVLIPFLEQNNFSPNTRTQKARHCGH

SEQ ID NO: 79 - protein, 70 aa, synthetic construct
  source 1..70: note = NKG2C ICD
MNKQRGTFSEVSLAQDPKRQQRKPKGNKSSISGTEQEIFQVELNLQNPSLNHQGIDKIYDCQGLLPPPEK

SEQ ID NO: 80 - protein, 23 aa, synthetic construct
  source 1..23: note = NKG2C TM
LTAEVLGIICIVLMATVLKTIVL

SEQ ID NO: 81 - protein, 23 aa, synthetic construct
  source 1..23: note = NKG2C hinge
IPFLEQNNFSPNTRTQKARHCGH

SEQ ID NO: 82 - protein, 116 aa, synthetic construct
  source 1..116: note = NKG2E ICD, TM, hinge
MSKQRGTFSEVSLAQDPKWQQRKPKGNKSSISGTEQEIFQVELNLQNASLNHQGIDKIYDCQGLLPPPEKLTAEVLGIICIVLMATVLKTIVLIPFLEQNNSSPNARTQKARHCGH

SEQ ID NO: 83 - protein, 70 aa, synthetic construct
  source 1..70: note = NKG2E ICD
MSKQRGTFSEVSLAQDPKWQQRKPKGNKSSISGTEQEIFQVELNLQNASLNHQGIDKIYDCQGLLPPPEK

SEQ ID NO: 84 - protein, 23 aa, synthetic construct
  source 1..23: note = NKG2E TM
LTAEVLGIICIVLMATVLKTIVL

SEQ ID NO: 85 - protein, 23 aa, synthetic construct
  source 1..23: note = NKG2E hinge
IPFLEQNNSSPNARTQKARHCGH

SEQ ID NO: 86 - protein, 128 aa, synthetic construct
  source 1..128: note = SAP
MDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 87 - protein, 132 aa, synthetic construct
  source 1..132: note = EAT2
MDLPYYHGRLTKQDCETLLLKEGVDGNFLLRDSESIPGVLCLCVSFKNIVYTYRIFREKHGYYRIQTAEGSPKQVFPSLKELISKFEKPNQGMVVHLLKPIKRTSPSLRWRGLKLELETFVNSNSDYVDVLP

SEQ ID NO: 88 - protein, 383 aa, synthetic construct
  source 1..383: note = MICA
MGLGPVFLLLAGIFPFAPPGAAAEPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 89 - protein, 383 aa, synthetic construct
  source 1..383: note = MICB
MGLGRVLLFLAVAFPFAPPAAAAEPHSLRYNLMVLSQDGSVQSGFLAEGHLDGQPFLRYDRQKRRAKPQGQWAENVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTDFPYVSAAMPCFVIIIILCVPCCKKKTSAAEGPELVSLQVLDQHPVGTGDHRDAAQLGFQPLMSATGSTGSTEGT

SEQ ID NO: 90 - protein, 358 aa, synthetic construct
  source 1..358: note = HLA-E
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 91 - protein, 266 aa, synthetic construct
  source 1..266: note = Membrane bound CD48
MALPVTALLLPLALLLHAARPHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLGKKDPWELRGAQGNWSCFEQRKAGGPIQPPCTVWWIYIWAPLAGTCGVLLLSLVIT

SEQ ID NO: 92 - protein, 603 aa, synthetic construct
  source 1..603: note = GPC3
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTETEKKIWHFKYPIFFLCIGLDLQIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 93 - protein, 313 aa, synthetic construct
  source 1..313: note = CD19
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHL

SEQ ID NO: 94 - protein, 254 aa, synthetic construct
  source 1..254: note = CD137L
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 95 - protein, 550 aa, synthetic construct
  source 1..550: note = LUC2
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 96 - protein, 219 aa, synthetic construct
  source 1..219: note = CD28 hinge/TM/ICD-CD3zeta in CAR1 in T CAR
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - protein, 243 aa, synthetic construct; identical to SEQ ID NO: 22
  source 1..243: note = 2B4 hinge/TM/ICD in CAR1

SEQ ID NO: 98 - protein, 331 aa, synthetic construct
  source 1..331: note = CD8 hinge-NKG2DTM-2B4ICD-CD3zeta in CAR2
PAKPTTTPAPRPPTPAPTIASQPLSLRPEACRPAAGGAVHTRGLDFACDPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 481 aa, synthetic construct
  source 1..481: note = CD8 hinge-NKG2DTM-2B4ICD-CD3zeta-P2A-SAP in CAR3
PAKPTTTPAPRPPTPAPTIASQPLSLRPEACRPAAGGAVHTRGLDFACDPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 100 - protein, 355 aa, synthetic construct
  source 1..355: note = ScFv-CD8 hinge-2B4TM/ICD-DAP10-CD3zeta in CAR4
PAKPTTTPAPRPPTPAPTIASQPLSLRPEACRPAAGGAVHTRGLDFACDFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 331 aa, synthetic construct
  source 1..331: note = CD8 hinge-2B4TM/ICD-CD3zeta in CAR4A
PAKPTTTPAPRPPTPAPTIASQPLSLRPEACRPAAGGAVHTRGLDFACDFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 361 aa, synthetic construct
  source 1..361: note = CD8 hinge-2B4TM-DAP10-2B4ICD-CD3zeta in CAR4B
PAKPTTTPAPRPPTPAPTIASQPLSLRPEACRPAAGGAVHTRGLDFACDFWVLVVVGGVLACYSLLVTVAFIIFWVLCARPRRSPAQEDGKVYINMPGRGWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 214 aa, synthetic construct
  source 1..214: note = CD8 hinge-2B4TM/ICD-DAP10 in CAR4C
PAKPTTTPAPRPPTPAPTIASQPLSLRPEACRPAAGGAVHTRGLDFACDFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSLCARPRRSPAQEDGKVYINMPGRG

SEQ ID NO: 104 - protein, 505 aa, synthetic construct
  source 1..505: note = CD8 hinge-2B4TM/ICD-DAP10-CD3zeta-P2A-SAP in CAR5
PAKPTTTPAPRPPTPAPTIASQPLSLRPEACRPAAGGAVHTRGLDFACDFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 105 - protein, 505 aa, synthetic construct
  source 1..505: note = CD8 hinge-2B4TM-DAP10-2B4ICD-CD3zeta-P2A-SAP in CAR5B
PAKPTTTPAPRPPTPAPTIASQPLSLRPEACRPAAGGAVHTRGLDFACDFLVIIVILSALFLGTLACFCVLCARPRRSPAQEDGKVYINMPGRGWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 106 - protein, 408 aa, synthetic construct
  source 1..408: note = 2B4 hinge/TM/ICD-DAP10-CD3zeta in CAR6
SLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - protein, 408 aa, synthetic construct
  source 1..408: note = 2B4 hinge/TM-DAP10-2B4ICD-CD3zeta in CAR6B
SLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVLCARPRRSPAQEDGKVYINMPGRGWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 558 aa, synthetic construct
  source 1..558: note = 2B4 hinge/TM/ICD-DAP10-CD3zeta-P2A-SAP in CAR8
SLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 109 - protein, 558 aa, synthetic construct
  source 1..558: note = 2B4 hinge/TM-DAP10-2B4ICD-CD3zeta-P2A-SAP in CAR8B
SLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVLCARPRRSPAQEDGKVYINMPGRGWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 110 - protein, 562 aa, synthetic construct
  source 1..562: note = 2B4 hinge/TM/ICD-DAP10-CD3zeta-P2A-EAT2 in CAR10
SLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDLPYYHGRLTKQDCETLLLKEGVDGNFLLRDSESIPGVLCLCVSFKNIVYTYRIFREKHGYYRIQTAEGSPKQVFPSLKELISKFEKPNQGMVVHLLKPIKRTSPSLRWRGLKLELETFVNSNSDYVDVLP

SEQ ID NO: 111 - protein, 389 aa, synthetic construct
  source 1..389: note = CD229 hinge/TM-DAP10-CD229ICD-CD3zeta in CAR11
PVSRSSHQFLSENICSGPERNTKLWIGLFLMVCLLCVGIFSWCIWLCARPRRSPAQEDGKVYINMPGRGKRKGRCSVPAFCSSQAEAPADTPEPTAGHTLYSVLSQGYEKLDTPLRPARQQPTPTSDSSSDSNLTTEEDEDRPEVHKPISGRYEVFDQVTQEGAGHDPAPEGQADYDPVTPYVTEVESVVGENTMYAQVFNLQGKTPVSQKEESSATIYCSIRKPQVVPPPQQNDLEIPESPTYENFTSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - protein, 539 aa, synthetic construct
  source 1..539: note = CD229 hinge/TM-DAP10-CD229ICD-CD3zeta-P2A-SAP in CAR12
PVSRSSHQFLSENICSGPERNTKLWIGLFLMVCLLCVGIFSWCIWLCARPRRSPAQEDGKVYINMPGRGKRKGRCSVPAFCSSQAEAPADTPEPTAGHTLYSVLSQGYEKLDTPLRPARQQPTPTSDSSSDSNLTTEEDEDRPEVHKPISGRYEVFDQVTQEGAGHDPAPEGQADYDPVTPYVTEVESVVGENTMYAQVFNLQGKTPVSQKEESSATIYCSIRKPQVVPPPQQNDLEIPESPTYENFTSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 113 - protein, 371 aa, synthetic construct
  source 1..371: note = NTB-A hinge/TM-DAP10-NTB-AICD-CD3zeta in CAR13
LRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTKMILFMVSGICIVFGFIILLLLVLCARPRRSPAQEDGKVYINMPGRGLRKRRDSLSLSTQRTQGPAESARNLEYVSVSPTNNTVYASVTHSNRETEIWTPRENDTITIYSTINHSKESKPTFSRATALDNVVSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 114 - protein, 521 aa, synthetic construct
  source 1..521: note = NTB-A hinge/TM-DAP10-NTB-AICD-CD3zeta-P2A-SAP in CAR14
LRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTKMILFMVSGICIVFGFIILLLLVLCARPRRSPAQEDGKVYINMPGRGLRKRRDSLSLSTQRTQGPAESARNLEYVSVSPTNNTVYASVTHSNRETEIWTPRENDTITIYSTINHSKESKPTFSRATALDNVVSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 115 - protein, 397 aa, synthetic construct
  source 1..397: note = CRACC hinge/TM-DAP10-CRACCICD-CD3zeta in CAR15
YVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLLCARPRRSPAQEDGKVYINMPGRGWFLKRERQEEYIEEKKRVDICRETPNICPHSGENTEYDTIPHTNRTILKEDPANTVYSTVEIPKKMENPHSLLTMPDTPRLFAYENVISFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - protein, 547 aa, synthetic construct
  source 1..547: note = CRACC hinge/TM-DAP10-CRACCICD-CD3zeta-P2A-SAP in CAR16
YVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLLCARPRRSPAQEDGKVYINMPGRGWFLKRERQEEYIEEKKRVDICRETPNICPHSGENTEYDTIPHTNRTILKEDPANTVYSTVEIPKKMENPHSLLTMPDTPRLFAYENVISFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 117 - protein, 551 aa, synthetic construct
  source 1..551: note = CRACC hinge/TM-DAP10-CRACCICD-CD3zeta-P2A-EAT2 in CAR16B
YVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLLCARPRRSPAQEDGKVYINMPGRGWFLKRERQEEYIEEKKRVDICRETPNICPHSGENTEYDTIPHTNRTILKEDPANTVYSTVEIPKKMENPHSLLTMPDTPRLFAYENVISFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDLPYYHGRLTKQDCETLLLKEGVDGNFLLRDSESIPGVLCLCVSFKNIVYTYRIFREKHGYYRIQTAEGSPKQVFPSLKELISKFEKPNQGMVVHLLKPIKRTSPSLRWRGLKLELETFVNSNSDYVDVLP

SEQ ID NO: 118 - protein, 313 aa, synthetic construct
  source 1..313: note = CD84 hinge/TM-DAP10-CD84ICD-CD3zeta in CAR17
PVSNNSDSISARQLCADIAMGFRTHHTGLLSVLAMFFLLVLILSSVFLFLCARPRRSPAQEDGKVYINMPGRGRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVISFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 463 aa, synthetic construct
  source 1..463: note = CD84 hinge/TM-DAP10-CD84ICD-CD3zeta-P2A-SAP in CAR18
PVSNNSDSISARQLCADIAMGFRTHHTGLLSVLAMFFLLVLILSSVFLFLCARPRRSPAQEDGKVYINMPGRGRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVISFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 120 - protein, 396 aa, synthetic construct
  source 1..396: note = CD2 hinge/TM-DAP10-CD2ICD-CD3zeta in CAR19
IFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITLCARPRRSPAQEDGKVYINMPGRGKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 546 aa, synthetic construct
  source 1..546: note = CD2 hinge/TM-DAP10-CD2ICD-CD3zeta-P2A-SAP in CAR20
IFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITLCARPRRSPAQEDGKVYINMPGRGKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 122 - protein, 333 aa, synthetic construct
  source 1..333: note = CD28H hinge/TM-DAP10-CD28HICD-CD3zeta in CAR21
AVEIPELEEAEGNITRLFVDPDDPTQNRNRIASFPGFLFVLLGVGSMGVAAIVWGAWLCARPRRSPAQEDGKVYINMPGRGFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEESFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 483 aa, synthetic construct
  source 1..483: note = CD28H hinge/TM-DAP10-CD28HICD-CD3zeta-P2A-SAP in CAR22
AVEIPELEEAEGNITRLFVDPDDPTQNRNRIASFPGFLFVLLGVGSMGVAAIVWGAWLCARPRRSPAQEDGKVYINMPGRGFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEESFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 124 - protein, 261 aa, synthetic construct
  source 1..261: note = DNAM1 hinge/TM-DAP10-DNAM1ICD-CD3zeta in CAR23
AEGKTDNQYTLFVAGGTVLLLLFVISITTIIVIFLLCARPRRSPAQEDGKVYINMPGRGNRRRRRERRDLFTESWDTQKAPNNYRSPISTSQPTNQSMDDTREDIYVNYPTFSRRPKTRVSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 411 aa, synthetic construct
  source 1..411: note = DNAM1 hinge/TM-DAP10-DNAM1ICD-CD3zeta-P2A-SAP in CAR24
AEGKTDNQYTLFVAGGTVLLLLFVISITTIIVIFLLCARPRRSPAQEDGKVYINMPGRGNRRRRRERRDLFTESWDTQKAPNNYRSPISTSQPTNQSMDDTREDIYVNYPTFSRRPKTRVSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 126 - protein, 253 aa, synthetic construct
  source 1..253: note = CD28 hinge/TM-DAP10 ICD-CD28 ICD-CD3zeta in CAR25
IHVKGKHLCPSPLFPGPSKPFWVLVVVGGVLACYSLLVTVAFIIFWVLCARPRRSPAQEDGKVYINMPGRGRSKRSRLLHSDYMNMTPRRPGPTRKHYQPYAPPRDFAAYRSSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 403 aa, synthetic construct
  source 1..403: note = CD28 hinge/TM-DAP10 ICD-CD28 ICD-CD3zeta-P2A-SAP in CAR26
IHVKGKHLCPSPLFPGPSKPFWVLVVVGGVLACYSLLVTVAFIIFWVLCARPRRSPAQEDGKVYINMPGRGRSKRSRLLHSDYMNMTPRRPGPTRKHYQPYAPPRDFAAYRSSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 128 - protein, 260 aa, synthetic construct
  source 1..260: note = 4-1BB hinge/TM-DAP10 ICD-4-1BB ICD-CD3zeta in CAR27
SPADLSPGASSVTPPAPAREPGHSPQIISFFLALTSTALLFLLFFLTLRFSVVLCARPRRSPAQEDGKVYINMPGRGKRGRKKLLYIFKQPFMRPVQTTQEEDGCSCRFPEEEEGGCELSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 410 aa, synthetic construct
  source 1..410: note = 4-1BB hinge/TM-DAP10 ICD-4-1BB ICD-CD3zeta-P2A-SAP in CAR28
SPADLSPGASSVTPPAPAREPGHSPQIISFFLALTSTALLFLLFFLTLRFSVVLCARPRRSPAQEDGKVYINMPGRGKRGRKKLLYIFKQPFMRPVQTTQEEDGCSCRFPEEEEGGCELSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 130 - protein, 275 aa, synthetic construct
  source 1..275: note = OX40 hinge/TM-DAP10 ICD-OX40 ICD-CD3zeta in CAR29
DRDPPATQPQETQGPPARPITVQPTEAWPRTSQGPSTRPVEVPGGRAVAAILGLGLVLGLLGPLAILLLCARPRRSPAQEDGKVYINMPGRGALYLLRRDQRLPPDAHKPPGGGSFRTPIQEEQADAHSTLAKISFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 425 aa, synthetic construct
  source 1..425: note = OX40 hinge/TM-DAP10 ICD-OX40 ICD-CD3zeta-P2A-SAP in CAR30
DRDPPATQPQETQGPPARPITVQPTEAWPRTSQGPSTRPVEVPGGRAVAAILGLGLVLGLLGPLAILLLCARPRRSPAQEDGKVYINMPGRGALYLLRRDQRLPPDAHKPPGGGSFRTPIQEEQADAHSTLAKISFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 132 - protein, 230 aa, synthetic construct
  source 1..230: note = KIR2DS1 hinge/TM-DAP10 ICD-CD3zeta in CAR31
SFRDSPYEWSKSSDPLLVSVTGNPSNSWPSPTEPSSETGNPRHLHVLIGTSVVKIPFTILLFFLLLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 380 aa, synthetic construct
  source 1..380: note = KIR2DS1 hinge/TM-DAP10 ICD-CD3zeta-P2A-SAP in CAR32
SFRDSPYEWSKSSDPLLVSVTGNPSNSWPSPTEPSSETGNPRHLHVLIGTSVVKIPFTILLFFLLLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 134 - protein, 230 aa, synthetic construct
  source 1..230: note = KIR2DS2 hinge/TM-DAP10 ICD-CD3zeta in CAR33
SFRDSPYEWSNSSDPLLVSVTGNPSNSWPSPTEPSSKTGNPRHLHVLIGTSVVKIPFTILLFFLLLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 380 aa, synthetic construct
  source 1..380: note = KIR2DS2 hinge/TM-DAP10 ICD-CD3zeta-P2A-SAP in CAR34
SFRDSPYEWSNSSDPLLVSVTGNPSNSWPSPTEPSSKTGNPRHLHVLIGTSVVKIPFTILLFFLLLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 136 - protein, 230 aa, synthetic construct
  source 1..230: note = KIR2DS3 hinge/TM-DAP10 ICD-CD3zeta in CAR35
SFHDSPYEWSKSSDPLLVSVTGNPSNSWPSPTEPSSKTGNPRHLHVLIGTSVVKLPFTILLFFLLLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 380 aa, synthetic construct
  source 1..380: note = KIR2DS3 hinge/TM-DAP10 ICD-CD3zeta-P2A-SAP in CAR36
SFHDSPYEWSKSSDPLLVSVTGNPSNSWPSPTEPSSKTGNPRHLHVLIGTSVVKLPFTILLFFLLLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 138 - protein, 230 aa, synthetic construct
  source 1..230: note = KIR2DS4 hinge/TM-DAP10 ICD-CD3zeta in CAR37
SFRDAPYEWSNSSDPLLVSVTGNPSNSWPSPTEPSSKTGNPRHLHVLIGTSVVKIPFTILLFFLLLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 380 aa, synthetic construct
  source 1..380: note = KIR2DS4 hinge/TM-DAP10 ICD-CD3zeta-P2A-SAP in CAR38
SFRDAPYEWSNSSDPLLVSVTGNPSNSWPSPTEPSSKTGNPRHLHVLIGTSVVKIPFTILLFFLLLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 140 - protein, 230 aa, synthetic construct
  source 1..230: note = KIR2DS5 hinge/TM-DAP10 ICD-CD3zeta in CAR39
SFRDSPYEWSKSSDPLLVSVTGNSSNSWPSPTEPSSETGNPRHLHVLIGTSVVKLPFTILLFFLLLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 380 aa, synthetic construct
  source 1..380: note = KIR2DS5 hinge/TM-DAP10 ICD-CD3zeta-P2A-SAP in CAR40
SFRDSPYEWSKSSDPLLVSVTGNSSNSWPSPTEPSSETGNPRHLHVLIGTSVVKLPFTILLFFLLLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 142 - protein, 211 aa, synthetic construct
  source 1..211: note = NKG2C hinge/TM-DAP10 ICD-CD3zeta in CAR41
IPFLEQNNFSPNTRTQKARHCGHLTAEVLGIICIVLMATVLKTIVLLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 361 aa, synthetic construct
  source 1..361: note = NKG2C hinge/TM-DAP10 ICD-CD3zeta-P2A-SAP in CAR42
IPFLEQNNFSPNTRTQKARHCGHLTAEVLGIICIVLMATVLKTIVLLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 144 - protein, 211 aa, synthetic construct
  source 1..211: note = NKG2E hinge/TM-DAP10 ICD-CD3zeta in CAR43
IPFLEQNNSSPNARTQKARHCGHLTAEVLGIICIVLMATVLKTIVLLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 361 aa, synthetic construct
  source 1..361: note = NKG2E hinge/TM-DAP10 ICD-CD3zeta-P2A-SAP in CAR44
IPFLEQNNSSPNARTQKARHCGHLTAEVLGIICIVLMATVLKTIVLLCARPRRSPAQEDGKVYINMPGRGSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMDAVAVYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEKKSSARSTQGTTGIREDPDVCLKAP

SEQ ID NO: 146 - skipped

SEQ ID NO: 147 - protein, 5 aa, synthetic construct
  source 1..5: note = Exemplary peptide linker
  REPEAT 1..5: note = GSGGS can be repeated n times, wherein n is an integer including, e.g., 1, 2, 3, 4, 5, and 6
GSGGS

SEQ ID NO: 148 - protein, 4 aa, synthetic construct
  source 1..4: note = Exemplary peptide linker
  REPEAT 1..4: note = GGGS can be repeated n times, wherein n is an integer including, e.g., 1, 2, 3, 4, 5, and 6
GGGS

SEQ ID NO: 149 - protein, 5 aa, synthetic construct
  source 1..5: note = Exemplary peptide linker
  REPEAT 1..5: note = GGGGS can be repeated n times, wherein n is an integer including, e.g., 1, 2, 3, 4, 5, and 6
GGGGS

SEQ ID NO: 150 - skipped

SEQ ID NO: 151 - protein, 4 aa, synthetic construct
  source 1..4: note = Exemplary peptide linker
GSGS

SEQ ID NO: 152 - protein, 6 aa, synthetic construct
  source 1..6: note = Exemplary peptide linker
GSGSGS

SEQ ID NO: 153 - protein, 4 aa, synthetic construct
  source 1..4: note = Exemplary peptide linker
GGGS

SEQ ID NO: 154 - protein, 8 aa, synthetic construct
  source 1..8: note = Exemplary peptide linker
GGGSGGGS

SEQ ID NO: 155 - protein, 5 aa, synthetic construct
  source 1..5: note = Exemplary peptide linker
GSGGS

SEQ ID NO: 156 - protein, 10 aa, synthetic construct
  source 1..10: note = Exemplary peptide linker
GSGGSGSGGS

SEQ ID NO: 157 - protein, 6 aa, synthetic construct
  source 1..6: note = Exemplary peptide linker
GSGGGS

SEQ ID NO: 158 - protein, 5 aa, synthetic construct
  source 1..5: note = Exemplary peptide linker
GGGGS

SEQ ID NO: 159 - protein, 10 aa, synthetic construct
  source 1..10: note = Exemplary peptide linker
GGGGSGGGGS

SEQ ID NO: 160 - protein, 4 aa, synthetic construct
  source 1..4: note = Exemplary peptide linker
GGRR

SEQ ID NO: 161 - protein, 24 aa, synthetic construct
  source 1..24: note = Exemplary peptide linker
GGGGSGGGGSGGGGGGSGSGGGGS

SEQ ID NO: 162 - protein, 8 aa, synthetic construct
  source 1..8: note = Exemplary peptide linker
GGRRGGGS

SEQ ID NO: 163 - protein, 244 aa, synthetic construct
  source 1..244: note = Anti-CD19 scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIK

SEQ ID NO: 164 - protein, 8 aa, synthetic construct
  source 1..8: note = VH CDR1 of anti-CD19 scFv as determined by software Geneious Biologics
GYAFSSYW

SEQ ID NO: 165 - protein, 8 aa, synthetic construct
  source 1..8: note = VH CDR2 of anti-CD19 scFv as determined by software Geneious Biologics
IYPGDGDT

SEQ ID NO: 166 - protein, 15 aa, synthetic construct
  source 1..15: note = VH CDR3 of anti-CD19 scFv as determined by software Geneious Biologics
ARKTISSVVDFYFDY

SEQ ID NO: 167 - protein, 6 aa, synthetic construct
  source 1..6: note = VL CDR1 of anti-CD19 scFv as determined by software Geneious Biologics
QNVGTN

SEQ ID NO: 168 - skipped

SEQ ID NO: 169 - protein, 9 aa, synthetic construct
  source 1..9: note = VL CDR3 of anti-CD19 scFv as determined by software Geneious Biologics
QQYNRYPYT

SEQ ID NO: 170 - protein, 242 aa, synthetic construct
  source 1..242: note = Anti-GPC3 scFv
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKR

SEQ ID NO: 171 - protein, 8 aa, synthetic construct
  source 1..8: note = VH CDR1 of anti-GPC3 scFv as determined by software Geneious Biologics
GYTFTDYE

SEQ ID NO: 172 - protein, 8 aa, synthetic construct
  source 1..8: note = VH CDR2 of anti-GPC3 scFv as determined by software Geneious Biologics
LDPKTGDT

SEQ ID NO: 173 - protein, 8 aa, synthetic construct
  source 1..8: note = VH CDR3 of anti-GPC3 scFv as determined by software Geneious Biologics
TRFYSYTY

SEQ ID NO: 174 - protein, 11 aa, synthetic construct
  source 1..11: note = VL CDR1 of anti-GPC3 scFv as determined by software Geneious Biologics
QSLVHSNGNTY

SEQ ID NO: 175 - skipped

SEQ ID NO: 176 - protein, 9 aa, synthetic construct
  source 1..9: note = VL CDR3 of anti-GPC3 scFv as determined by software Geneious Biologics
SQNTHVPPT

SEQ ID NO: 177 - protein, 25 aa, synthetic construct
  source 1..25: note = Reversed sequence of DAP10 ICD
GRGPMNIYVKGDEDQAPSRRPRACL

SEQ ID NO: 178 - protein, 128 aa, synthetic construct
  source 1..128: note = Reversed sequence of SAP
PAKLCVDPDERIGTTGQTSRASSKKEVPYQLPIVIGQDPKQFASILNKIKRFYRKHVGPATEASWSGTETQSVRYTYIYGHYLVCLCYVGPVSESDRLLYSGDLGTALLLKEGTERSIKGHYVAVADM

SEQ ID NO: 179 - protein, 132 aa, synthetic construct
  source 1..132: note = Reversed sequence of EAT2
PLVDVYDSNSNVFTELELKLGRWRLSPSTRKIPKLLHVVMGQNPKEFKSILEKLSPFVQKPSGEATQIRYYGHKERFIRYTYVINKFSVCLCLVGPISESDRLLFNGDVGEKLLLTECDQKTLRGHYYPLDM